SEQUENCE LISTING (16 sequences)

SEQ ID NO: 1 - DNA, 1905 bp, Artificial Sequence
  Synthetic sequence based on Bacillus thuringiensis
atgggacacaatgttttgaataacgaaagaaatactgtttgtgaagcacacaatgttgcagtgagagatccattctctttcgaacacaagagtcttaatacagttgaaaaggagtggacagagtggaagagaaatgatcactcactttatgtggatcctattgtcggaacagttgcctctttcttgcttaagaaggttggaagtttagtgggtaagagaatcttatcagagttgagatcattgatcttcccttctggttctacaaatttgatgcaagatatcttaagagagaccgagaaattcttgaatcaaagacttaataccgatactctcgcccgtgttaatgctgagctaacaggtctgcaagcaaacgtggaggagttcaacaggcaagtcgacaacttcttaaaccctaacagaaacgccttaccattatcaatcacctctagtgtgaacactatgcagcagttattcttgaacaggcttcctcagttccagatccagggctatcagctcctactcttaccattgttcgctcaggctgctaaccttcacctctccttcatccgtgacgttatcctaaacgctgacgaatggggaatcagcgcagcaactctgcgaacttaccgcaaccatcttagaaactacactagagactactcgaactactgcattaacacttaccagacagctttcaggggtttgaacacacgtcttcacgacatgctcgagttccgaacatacatgttcctaaacgtcttcgaatacgtgtcaatatggtcactgttcaaataccagtcattattggtctctagtggcgcaaacctttacgcctccggaagcggaccacagcaaactcagtcgttcacatcacaagactggccatttctctactctctattccaagtaaacagtaactatgttctgaacggattctccggagcgcgcttaagccaaacctttccaaacatcgtgggtttgccaggaacgactactactcatgctcttctcgctgctagagtcaactactcgggaggcgtatcatctggcgacattggggctagtcctttcaaccagaacttctcctgtagcacatttctaccaccgctgttaacaccatttgttaggtcgtggttggactctggatctgatcgtggaggagtgaacaccgtcacgaactggcaaactgaaagctttgaaacaacccttggtctccgatccggcgcattcacggcccgcgggaacagcaactactttcctgactatttcataagaaatatatcgggagtaccactagttgtgaggaacgaagatctgcgtcgaccgttacattataatcaaatacgcaatatagaatcaccatctggaactcctggcggtgcgagagcttatatggtcagtgtacataataggaagaacaacatatatgcagttcatgaaaatggaacaatgatacatttggctcccgaagattataccggtttcacgatatcaccgatacatgcgactcaagtgaacaatcaaacacgtacctttatttcagaaaaatttggcaatcaaggggattcgcttcgatttgaacaatcaaatacgactgctcgctatacactcagaggaaatggaaattcttataatctatatctgagggtcagttccattggcaatagcaccattcgtgtaacgattaatgggcgagtttatactgcaacaaatgtgaataccacgactaacaatgatggagtcaatgataatggtgcccgcttctcagatattaatattggcaatgtagttgcgtctgataatagtaacgtgccacttgatattaatgtcacattgaattccgggacccaatttgatcttatgaatattatgtttgtacctacgaatctcccaccgctatattag

SEQ ID NO: 2 - protein, 634 aa, Artificial sequence
  Synthetic sequence based on Bacillus thuringiensis
MGHNVLNNERNTVCEAHNVAVRDPFSFEHKSLNTVEKEWTEWKRNDHSLYVDPIVGTVASFLLKKVGSLVGKRILSELRSLIFPSGSTNLMQDILRETEKFLNQRLNTDTLARVNAELTGLQANVEEFNRQVDNFLNPNRNALPLSITSSVNTMQQLFLNRLPQFQIQGYQLLLLPLFAQAANLHLSFIRDVILNADEWGISAATLRTYRNHLRNYTRDYSNYCINTYQTAFRGLNTRLHDMLEFRTYMFLNVFEYVSIWSLFKYQSLLVSSGANLYASGSGPQQTQSFTSQDWPFLYSLFQVNSNYVLNGFSGARLSQTFPNIVGLPGTTTTHALLAARVNYSGGVSSGDIGASPFNQNFSCSTFLPPLLTPFVRSWLDSGSDRGGVNTVTNWQTESFETTLGLRSGAFTARGNSNYFPDYFIRNISGVPLVVRNEDLRRPLHYNQIRNIESPSGTPGGARAYMVSVHNRKNNIYAVHENGTMIHLAPEDYTGFTISPIHATQVNNQTRTFISEKFGNQGDSLRFEQSNTTARYTLRGNGNSYNLYLRVSSIGNSTIRVTINGRVYTATNVNTTTNNDGVNDNGARFSDINIGNVVASDNSNVPLDINVTLNSGTQFDLMNIMFVPTNLPPLY

SEQ ID NO: 3 - DNA, 1101 bp, Artificial Sequence
  Synthetic sequence based on Rhyzopus oryzae
gttccagtctcaggaaagtctggttcttcaactacagctgtttctgcatcagataactccgccctacctcctctgatttcatcgagatgcgctccaccttcaaacaagggatcaaagtcagatttgcaagctgaaccatattatatgcaaaaaaacaccgagtggtacgagtctcacggtggcaacttaactagtatcggaaagagagatgataacttggttggaggaatgacacttgaccttccatccgacgctccaccaatcagcctatcgggttcaaccaactctgcaagtgacggaggtaaggtcgttgcagctactactgcacaaatccaggagttcacaaagtacgccggaatcgcggctacagcttactgcagatccgttgtgcctgggaacaagtgggactgtgttcagtgccagaaatgggtgcccgacggaaagattataactacattcaccagcctgttatcggacacgaatggatacgtcttgaggtcagataaacagaaaactatctaccttgtattccgtggtactaactctttccgaagtgctattactgacatagtcttcaatttctccgattataaaccggttaaaggcgcaaaggttcacgccgggtttctcagctcgtacgagcaagtggtcaatgattatttcccagtagttcaggaacaactaacagcgaatcctacctataaagtgatagtcacgggacattcactgggtggcgctcaagcattattggccgggatggatctttatcaacgcgagcccagactctctccgaagaatctaagtatattcactgtaggaggtccaagggttggcaatcctacatttgcgtattatgtggaatccaccgggataccctttcaacgcacggtccacaaacgagatatagtaccgcatgttccacctcaaagctttggatttctgcatcccggtgtggaatcatggattaaatctggcacttcaaatgtccaaatttgtacatccgaaattgaaaccaaagattgtagcaattcaattgtgccgtttacgtctttattggatcatcttagttactttgatattaatgaagggtcctgtctctag

SEQ ID NO: 4 - protein, 366 aa, Artificial Sequence
  Synthetic sequence based on Rhyzopus oryzae
VPVSGKSGSSTTAVSASDNSALPPLISSRCAPPSNKGSKSDLQAEPYYMQKNTEWYESHGGNLTSIGKRDDNLVGGMTLDLPSDAPPISLSGSTNSASDGGKVVAATTAQIQEFTKYAGIAATAYCRSVVPGNKWDCVQCQKWVPDGKIITTFTSLLSDTNGYVLRSDKQKTIYLVFRGTNSFRSAITDIVFNFSDYKPVKGAKVHAGFLSSYEQVVNDYFPVVQEQLTANPTYKVIVTGHSLGGAQALLAGMDLYQREPRLSPKNLSIFTVGGPRVGNPTFAYYVESTGIPFQRTVHKRDIVPHVPPQSFGFLHPGVESWIKSGTSNVQICTSEIETKDCSNSIVPFTSLLDHLSYFDINEGSCL

SEQ ID NO: 5 - DNA, 72 bp, Hordeum leporinum
atggcaaataagcatttgtcacttagtttattcttggtcttattgggactttcagcatctctcgccagtgga

SEQ ID NO: 6 - protein, 24 aa, Hordeum leporinum
MANKHLSLSLFLVLLGLSASLASG

SEQ ID NO: 7 - DNA, 3468 bp, Artificial Sequence
  Synthetic sequence based on Bacillus thuringiensis
atggataacaatccgaacatcaatgaatgcattccttataattgtttaagtaaccctgaagtagaagtattaggtggagaaagaatagaaactggttacaccccaatcgatatttccttgtcgctaacgcaatttcttttgagtgaatttgttcccggtgctggatttgtgttaggactagttgatataatatggggaatttttggtccctctcaatgggacgcatttcttgtacaaattgaacagttaattaaccaaagaatagaagaattcgctaggaaccaagccatttctagattagaaggactaagcaatctttatcaaatttacgcagaatcttttagagagtgggaagcagatcctactaatccagcattaagagaagagatgcgtattcaattcaatgacatgaacagtgcccttacaaccgctattcctctttttgcagttcaaaattatcaagttcctcttttatcagtatatgttcaagctgcaaatttacatttatcagttttgagagatgtttcagtgtttggacaaaggtggggatttgatgccgcgactatcaatagtcgttataatgatttaactaggcttattggcaactatacagatcatgctgtacgctggtacaatacgggattagagcgtgtatggggaccggattctagagattggataagatataatcaatttagaagagaattaacactaactgtattagatatcgtttctctatttccgaactatgatagtagaacgtatccaattcgaacagtttcccaattaacaagagaaatttatacaaacccagtattagaaaattttgatggtagttttcgaggctcggctcagggcatagaaggaagtattaggagtccacatttgatggatatacttaacagtataaccatctatacggatgctcatagaggagaatattattggtcagggcatcaaataatggcttctcctgtagggttttcggggccagaattcacttttccgctatatggaactatgggaaatgcagctccacaacaacgtattgttgctcaactaggtcagggcgtgtatagaacattatcgtccactttatatagaagaccttttaatatagggataaataatcaacaactatctgttcttgacgggacagaatttgcttatggaacctcctcaaatttgccatccgctgtatacagaaaaagcggaacggtagattcgctggatgaaataccgccacagaataacaacgtgccacctaggcaaggatttagtcatcgattaagccatgtttcaatgtttcgttcaggctttagtaatagtagtgtaagtataataagagctcctatgttctcttggatacatcgtagtgctgaatttaataatataattccttcatcacaaattacacaaatacctttaacaaaatctactaatcttggctctggaacttctgtcgttaaaggaccaggatttacaggaggagatattcttcgaagaacttcacctggccagatttcaaccttaagagtaaatattactgcaccattatcacaaagatatcgggtaagaattcgctacgcttctaccacaaatttacaattccatacatcaattgacggaagacctattaatcaggggaatttttcagcaactatgagtagtgggagtaatttacagtccggaagctttaggactgtaggttttactactccgtttaacttttcaaatggatcaagtgtatttacgttaagtgctcatgtcttcaattcaggcaatgaagtttatatagatcgaattgaatttgttccggcagaagtaacctttgaggcagaatatgatttagaaagagcacaaaaggcggtgaatgagctgtttacttcttccaatcaaatcgggttaaaaacagatgtgacggattatcatattgatcaagtatccaatttagttgagtgtttatctgatgaattttgtctggatgaaaaaaaagaattgtccgagaaagtcaaacatgcgaagcgacttagtgatgagcggaatttacttcaagatccaaactttagagggatcaatagacaactagaccgtggctggagaggaagtacggatattaccatccaaggaggcgatgacgtattcaaagagaattacgttacgctattgggtacctttgatgagtgctatccaacgtatttatatcaaaaaatagatgagtcgaaattaaaagcctatacccgttaccaattaagagggtatatcgaagatagtcaagacttagaaatctatttaattcgctacaatgccaaacacgaaacagtaaatgtgccaggtacgggttccttatggccgctttcagccccaagtccaatcggaaaatgtgcccatcattcccatcatttctccttggacattgatgttggatgtacagacttaaatgaggacttaggtgtatgggtgatattcaagattaagacgcaagatggccatgcaagactaggaaatctagaatttctcgaagagaaaccattagtaggagaagcactagctcgtgtgaaaagagcggagaaaaaatggagagacaaacgtgaaaaattggaatgggaaacaaatattgtttataaagaggcaaaagaatctgtagatgctttatttgtaaactctcaatatgatagattacaagcggataccaacatcgcgatgattcatgcggcagataaacgcgttcatagcattcgagaagcttatctgcctgagctgtctgtgattccgggtgtcaatgcggctatttttgaagaattagaagggcgtattttcactgcattctccctatatgatgcgagaaatgtcattaaaaatggtgattttaataatggcttatcctgctggaacgtgaaagggcatgtagatgtagaagaacaaaacaaccaccgttcggtccttgttgttccggaatgggaagcagaagtgtcacaagaagttcgtgtctgtccgggtcgtggctatatccttcgtgtcacagcgtacaaggagggatatggagaaggttgcgtaaccattcatgagatcgagaacaatacagacgaactgaagtttagcaactgtgtagaagaggaagtatatccaaacaacacggtaacgtgtaatgattatactgcgactcaagaagaatatgagggtacgtacacttctcgtaatcgaggatatgacggagcctatgaaagcaattcttctgtaccagctgattatgcatcagcctatgaagaaaaagcatatacagatggacgaagagacaatccttgtgaatctaacagaggatatggggattacacaccactaccagctggctatgtgacaaaagaattagagtacttcccagaaaccgataaggtatggattgagatcggagaaacggaaggaacattcatcgtggacagcgtggaattacttcttatggaggaataa

SEQ ID NO: 8 - DNA, 3468 bp, Artificial Sequence
  Synthetic sequence based on Bacillus thuringiensis
atggacaacaaccccaacatcaacgagtgcatcccctacaactgcctgagcaaccccgaggtggaggtgctgggcggcgagcgcatcgagaccggctacacccccatcgacatcagcctgagcctgacccagttcctgctgagcgagttcgtgcccggcgccggcttcgtgctgggcctggtggacatcatctggggcatcttcggccccagccagtgggacgccttcctggtgcagatcgagcagctgatcaaccagcgcatcgaggagttcgcccgcaaccaggccatcagccgcctggagggcctgagcaacctgtaccagatctacgccgagagcttccgcgagtgggaggccgaccccaccaaccccgccctgcgcgaggagatgcgcatccagttcaacgacatgaacagcgccctgaccaccgccatccccctgttcgccgtgcagaactaccaggtgcccctgctgagcgtgtacgtgcaggccgccaacctgcacctgagcgtgctgcgcgacgtgagcgtgttcggccagcgctggggcttcgacgccgccaccatcaacagccgctacaacgacctgacccgcctgatcggcaactacaccgaccacgccgtgcgctggtacaacaccggcctggagcgcgtgtggggccccgacagccgcgactggatccgctacaaccagttccgccgcgagctgaccctgaccgtgctggacatcgtgagcctgttccccaactacgacagccgcacctaccccatccgcaccgtgagccagctgacccgcgagatctacaccaaccccgtgctggagaacttcgacggcagcttccgcggcagcgcccagggcatcgagggcagcatccgcagcccccacctgatggacatcctgaacagcatcaccatctacaccgacgcccaccgcggcgagtactactggagcggccaccagatcatggccagccccgtgggcttcagcggccccgagttcaccttccccctgtacggcaccatgggcaacgccgccccccagcagcgcatcgtggcccagctgggccagggcgtgtaccgcaccctgagcagcaccctgtaccgccgccccttcaacatcggcatcaacaaccagcagctgagcgtgctggacggcaccgagttcgcctacggcaccagcagcaacctgcccagcgccgtgtaccgcaagagcggcaccgtggacagcctggacgagatccccccccagaacaacaacgtgcccccccgccagggcttcagccaccgcctgagccacgtgagcatgttccgcagcggcttcagcaacagcagcgtgagcatcatccgcgcccccatgttcagctggatccaccgcagcgccgagttcaacaacatcatccccagcagccagatcacccagatccccctgaccaagagcaccaacctgggcagcggcaccagcgtggtgaagggccccggcttcaccggcggcgacatcctgcgccgcaccagccccggccagatcagcaccctgcgcgtgaacatcaccgcccccctgagccagcgctaccgcgtgcgcatccgctacgccagcaccaccaacctgcagttccacaccagcatcgacggccgccccatcaaccagggcaacttcagcgccaccatgagcagcggcagcaacctgcagagcggcagcttccgcaccgtgggcttcaccacccccttcaacttcagcaacggcagcagcgtgttcaccctgagcgcccacgtgttcaacagcggcaacgaggtgtacatcgaccgcatcgagttcgtgcccgccgaggtgaccttcgaggccgagtacgacctggagcgcgcccagaaggccgtgaacgagctgttcaccagcagcaaccagatcggcctgaagaccgacgtgaccgactaccacatcgaccaggtgagcaacctggtggagtgcctgagcgacgagttctgcctggacgagaagaaggagctgagcgagaaggtgaagcacgccaagcgcctgagcgacgagcgcaacctgctgcaggaccccaacttccgcggcatcaaccgccagctggaccgcggctggcgcggcagcaccgacatcaccatccagggcggcgacgacgtgttcaaggagaactacgtgaccctgctgggcaccttcgacgagtgctaccccacctacctgtaccagaagatcgacgagagcaagctgaaggcctacacccgctaccagctgcgcggctacatcgaggacagccaggacctggagatctacctgatccgctacaacgccaagcacgagaccgtgaacgtgcccggcaccggcagcctgtggcccctgagcgcccccagccccatcggcaagtgcgcccaccacagccaccacttcagcctggacatcgacgtgggctgcaccgacctgaacgaggacctgggcgtgtgggtgatcttcaagatcaagacccaggacggccacgcccgcctgggcaacctggagttcctggaggagaagcccctggtgggcgaggccctggcccgcgtgaagcgcgccgagaagaagtggcgcgacaagcgcgagaagctggagtgggagaccaacatcgtgtacaaggaggccaaggagagcgtggacgccctgttcgtgaacagccagtacgaccgcctgcaggccgacaccaacatcgccatgatccacgccgccgacaagcgcgtgcacagcatccgcgaggcctacctgcccgagctgagcgtgatccccggcgtgaacgccgccatcttcgaggagctggagggccgcatcttcaccgccttcagcctgtacgacgcccgcaacgtgatcaagaacggcgacttcaacaacggcctgagctgctggaacgtgaagggccacgtggacgtggaggagcagaacaaccaccgcagcgtgctggtggtgcccgagtgggaggccgaggtgagccaggaggtgcgcgtgtgccccggccgcggctacatcctgcgcgtgaccgcctacaaggagggctacggcgagggctgcgtgaccatccacgagatcgagaacaacaccgacgagctgaagttcagcaactgcgtggaggaggaggtgtaccccaacaacaccgtgacctgcaacgactacaccgccacccaggaggagtacgagggcacctacaccagccgcaaccgcggctacgacggcgcctacgagagcaacagcagcgtgcccgccgactacgccagcgcctacgaggagaaggcctacaccgacggccgccgcgacaacccctgcgagagcaaccgcggctacggcgactacacccccctgcccgccggctacgtgaccaaggagctggagtacttccccgagaccgacaaggtgtggatcgagatcggcgagaccgagggcaccttcatcgtggacagcgtggagctgctgctgatggaggagtag

SEQ ID NO: 9 - DNA, 1947 bp, Artificial Sequence
  Synthetic sequence based on Bacillus thuringiensis
atggacaacaaccccaacatcaacgagtgcatcccctacaactgcctgagcaaccccgaggtggaggtgctgggcggcgagcgcatcgagaccggctacacccccatcgacatcagcctgagcctgacccagttcctgctgagcgagttcgtgcccggcgccggcttcgtgctgggcctggtggacatcatctggggcatcttcggccccagccagtgggacgccttcctggtgcagatcgagcagctgatcaaccagcgcatcgaggagttcgcccgcaaccaggccatcagccgcctggagggcctgagcaacctgtaccaaatctacgccgagagcttccgcgagtgggaggccgaccccaccaaccccgccctgcgcgaggagatgcgcatccagttcaacgacatgaacagcgccctgaccaccgccatccccctgttcgccgtgcagaactaccaggtgcccctgctgagcgtgtacgtgcaggccgccaacctgcacctgagcgtgctgcgcgacgtcagcgtgttcggccagcgctggggcttcgacgccgccaccatcaacagccgctacaacgacctgacccgcctgatcggcaactacaccgaccacgccgtgcgctggtacaacaccggcctggagcgcgtgtggggtcccgacagccgcgactggatcaggtacaaccagttccgccgcgagctgaccctgaccgtgctggacatcgtgagcctgttccccaactacgacagccgcacctaccccatccgcaccgtgagccagctgacccgcgagatttacaccaaccccgtgctggagaacttcgacggcagcttccgcggcagcgcccagggcatcgagggcagcatccgcagcccccacctgatggacatcctgaacagcatcaccatctacaccgacgcccaccgcggcgagtactactggagcggccaccagatcatggccagccccgtcggcttcagcggccccgagttcaccttccccctgtacggcaccatgggcaacgctgcacctcagcagcgcatcgtggcacagctgggccagggagtgtaccgcaccctgagcagcaccctgtaccgtcgacctttcaacatcggcatcaacaaccagcagctgagcgtgctggacggcaccgagttcgcctacggcaccagcagcaacctgcccagcgccgtgtaccgcaagagcggcaccgtggacagcctggacgagatcccccctcagaacaacaacgtgccacctcgacagggcttcagccaccgtctgagccacgtgagcatgttccgcagtggcttcagcaacagcagcgtgagcatcatccgtgcacctatgttcagctggattcaccgcagtgccgagttcaacaacatcatccccagcagccaaatcacccagatccccctgaccaagagcaccaacctgggcagcggcaccagcgtggtgaagggccccggcttcaccggcggcgacatcctgcgccgcaccagccccggccagatcagcaccctgcgcgtgaacatcaccgcccccctgagccagcgctaccgcgtccgcatccgctacgccagcaccaccaacctgcagttccacaccagcatcgacggccgccccatcaaccagggcaacttcagcgccaccatgagcagcggcagcaacctgcagagcggcagcttccgcaccgtgggcttcaccacccccttcaacttcagcaacggcagcagcgtgttcaccctgagcgcccacgtgttcaacagcggcaacgaggtgtacatcgaccgcatcgagttcgtgcccgccgaggtgaccttcgaggccgagtacgacctggagagggctcagaaggccgtgaacgagctgttcaccagcagcaaccagatcggcctgaagaccgacgtgaccgactaccacatcgatcaggtgtag

SEQ ID NO: 10 - DNA, 3468 bp, Artificial Sequence
  Synthetic sequence based on Bacillus thuringiensis
atggacaacaaccccaacatcaacgagtgcatcccctacaactgcctgagcaaccccgaggtggaggtgctgggcggcgagcgcatcgagaccggctacacccccatcgacatcagcctgagcctgacccagttcctgctgagcgagttcgtgcccggcgccggcttcgtgctgggcctggtggacatcatctggggcatcttcggccccagccagtgggacgccttcctggtgcagatcgagcagctgatcaaccagcgcatcgaggagttcgcccgcaaccaggccatcagccgcctggagggcctgagcaacctgtaccaaatctacgccgagagcttccgcgagtgggaggccgaccccaccaaccccgccctgcgcgaggagatgcgcatccagttcaacgacatgaacagcgccctgaccaccgccatccccctgttcgccgtgcagaactaccaggtgcccctgctgagcgtgtacgtgcaggccgccaacctgcacctgagcgtgctgcgcgacgtcagcgtgttcggccagcgctggggcttcgacgccgccaccatcaacagccgctacaacgacctgacccgcctgatcggcaactacaccgaccacgccgtgcgctggtacaacaccggcctggagcgcgtgtggggtcccgacagccgcgactggatcaggtacaaccagttccgccgcgagctgaccctgaccgtgctggacatcgtgagcctgttccccaactacgacagccgcacctaccccatccgcaccgtgagccagctgacccgcgagatttacaccaaccccgtgctggagaacttcgacggcagcttccgcggcagcgcccagggcatcgagggcagcatccgcagcccccacctgatggacatcctgaacagcatcaccatctacaccgacgcccaccgcggcgagtactactggagcggccaccagatcatggccagccccgtcggcttcagcggccccgagttcaccttccccctgtacggcaccatgggcaacgctgcacctcagcagcgcatcgtggcacagctgggccagggagtgtaccgcaccctgagcagcaccctgtaccgtcgacctttcaacatcggcatcaacaaccagcagctgagcgtgctggacggcaccgagttcgcctacggcaccagcagcaacctgcccagcgccgtgtaccgcaagagcggcaccgtggacagcctggacgagatcccccctcagaacaacaacgtgccacctcgacagggcttcagccaccgtctgagccacgtgagcatgttccgcagtggcttcagcaacagcagcgtgagcatcatccgtgcacctatgttcagctggattcaccgcagtgccgagttcaacaacatcatccccagcagccagatcacccagatccccctgaccaagagcaccaacctgggcagcggcaccagcgtggtgaagggccccggcttcaccggcggcgacatcctgcgccgcaccagccccggccagatcagcaccctgcgcgtgaacatcaccgcccccctgagccagcgctaccgcgtccgcatccgctacgccagcaccaccaacctgcagttccacaccagcatcgacggccgccccatcaaccagggcaacttcagcgccaccatgagcagcggcagcaacctgcagagcggcagcttccgcaccgtgggcttcaccacccccttcaacttcagcaacggcagcagcgtgttcaccctgagcgcccacgtgttcaacagcggcaacgaggtgtacatcgaccgcatcgagttcgtgcccgccgaggtgaccttcgaggccgagtacgacctggagagggctcagaaggccgtgaacgagctgttcaccagcagcaaccagatcggcctgaagaccgacgtgaccgactaccacatcgatcaggtgagcaacctggtggagtgcctgagcgacgagttctgcctggacgagaagaaggagctgagcgagaaggtgaagcacgccaagcgcctgagcgacgagcgcaacctgctgcaggaccccaacttccgcggcatcaaccgccagctggaccgcggctggcgcggcagcaccgacatcaccatccagggcggcgacgacgtgttcaaggagaactacgtgaccctgctgggcaccttcgacgagtgctaccccacctacctgtaccagaagatcgacgagagcaagctgaaggcctacacccgctaccagctgcgcggctacatcgaggacagccaggacctggagatctacctgatccgctacaacgccaagcacgagaccgtgaacgtgcccggcaccggcagcctgtggcccctgagcgcccccagccccatcggcaagtgcgcccaccacagccaccacttcagcctggacatcgacgtgggctgcaccgacctgaacgaggacctgggcgtgtgggtgatcttcaagatcaagacccaggacggccacgcccgcctgggcaacctggagttcctggaggagaagcccctggtgggcgaggccctggcccgcgtgaagcgcgccgagaagaagtggcgcgacaagcgcgagaagctggagtgggagaccaacatcgtgtacaaggaggccaaggagagcgtggacgccctgttcgtgaacagccagtacgaccgcctgcaggccgacaccaacatcgccatgatccacgccgccgacaagcgcgtgcacagcattcgcgaggcctacctgcccgagctgagcgtgatccccggcgtgaacgccgccatcttcgaggagctggagggccgcatcttcaccgccttcagcctgtacgacgcccgcaacgtgatcaagaacggcgacttcaacaacggcctgagctgctggaacgtgaagggccacgtggacgtggaggagcagaacaaccaccgcagcgtgctggtggtgcccgagtgggaggccgaggtgagccaggaggtgcgcgtgtgccccggccgcggctacatcctgcgcgtgaccgcctacaaggagggctacggcgagggctgcgtgaccatccacgagatcgagaacaacaccgacgagctcaagttcagcaactgcgtggaggaggaggtgtaccccaacaacaccgtgacctgcaacgactacaccgccacccaggaggagtacgagggcacctacaccagccgcaaccgcggctacgacggcgcctacgagagcaacagcagcgtgcccgccgactacgccagcgcctacgaggagaaggcctacaccgacggccgccgcgacaacccctgcgagagcaaccgcggctacggcgactacacccccctgcccgccggctacgtgaccaaggagctggagtacttccccgagaccgacaaggtgtggatcgagatcggcgagaccgagggcaccttcatcgtggacagcgtggagctgctgctgatggaggagtag

SEQ ID NO: 11 - DNA, 1845 bp, Artificial Sequence
  Synthetic sequence based on Bacillus thuringiensis
atggacaacaacccaaacatcaacgaatgcattccatacaactgcttgagtaacccagaagttgaagtacttggtggagaacgcattgaaaccggttacactcccatcgacatctccttgtccttgacacagtttctgctcagcgagttcgtgccaggtgctgggttcgttctcggactagttgacatcatctggggtatctttggtccatctcaatgggatgcattcctggtgcaaattgagcagttgatcaaccagaggatcgaagagttcgccaggaaccaggccatctctaggttggaaggattgagcaatctctaccaaatctatgcagagagcttcagagagtgggaagccgatcctactaacccagctctccgcgaggaaatgcgtattcaattcaacgacatgaacagcgccttgaccacagctatcccattgttcgcagtccagaactaccaagttcctctcttgtccgtgtacgttcaagcagctaatcttcacctcagcgtgcttcgagacgttagcgtgtttgggcaaaggtggggattcgatgctgcaaccatcaatagccgttacaacgaccttactaggctgattggaaactacaccgaccacgctgttcgttggtacaacactggcttggagcgtgtctggggtcctgattctagagattggattagatacaaccagttcaggagagaattgaccctcacagttttggacattgtgtctctcttcccgaactatgactccagaacctaccctatccgtacagtgtcccaacttaccagagaaatctatactaacccagttcttgagaacttcgacggtagcttccgtggttctgcccaaggtatcgaaggctccatcaggagcccacacttgatggacatcttgaacagcataactatctacagcgatgctcacagaggagagtattactggtctggacaccagatcatggcctctccagttggattcagcgggcccgagtttacctttcctctctatggaactatgggaaacgccgctccacaacaacgtatcgttgctcaactaggtcagggtgtctacagaaccttgtcttccaccttgtacagaagacccttcaatatcggtatcaacaaccagcaactttccgttcttgacggaacagagttcgcctatggaacctcttctaacttgccatccgctgtttacagaaagagcggaaccgttgattccttggacgaaatcccaccacagaacaacaatgtgccacccaggcaaggattctcccacaggttgagccacgtgtccatgttccgttccggattcagcaacagttccgtgagcatcatcagagctcctatgttctcatggattcatcgtagtgctgagttcaacaatatcattccttcctctcaaatcacccaaatcccattgaccaagtctactaaccttggatctggaacttctgtcgtgaaaggaccaggcttcacaggaggtgatattcttagaagaacttctcctggccagattagcaccctcagagttaacatcactgcaccactttctcaaagatatcgtgtcaggattcgttacgcatctaccactaacttgcaattccacacctccatcgacggaaggcctatcaatcagggtaacttctccgcaaccatgtcaagcggcagcaacttgcaatccggcagcttcagaaccgtcggtttcactactcctttcaacttctctaacggatcaagcgttttcacccttagcgctcatgtgttcaattctggcaatgaagtgtacattgaccgtattgagtttgtgcctgccgaagttaccttcgaggctgagtac

SEQ ID NO: 12 - DNA, 1833 bp, Artificial Sequence
  Synthetic sequence based on Bacillus thuringiensis
atggctgcagacaacaacacggaggccctcgatagctctaccaccaaagatgtcattcagaagggcatctccgttgtgggtgatctccttggcgttgttggtttcccctttggtggtgcccttgtttcgttctacactaactttctgaatactatttggcccagcgaagacccttggaaggcttttatggagcaagtggaagctttgatggatcagaagatcgctgattatgcaaagaacaaagctcttgctgagctccagggccttcagaacaacgtcgaagattatgtgagtgcactgagttcatggcaaaagaatcctgtgtcctcacgaaatccacatagccaggggcgcataagggagctgttctctcaagcagaaagtcacttccggaattcaatgccttcctttgccatctctgggtacgaggttctctttcttacaacctacgctcaagctgccaacacacatctgttcttactaaaagacgctcaaatctatggtgaagaatggggatacgagaaagaagatatcgctgagttctacaagcgtcaactaaaacttactcaagagtatactgaccactgtgtcaaatggtataatgttggattggataagttgagaggttcatcttatgaatcttgggtaaactttaaccggtaccgcagagagatgacattgacagtgctcgacttgattgcactatttccattgtatgatgttcgactctacccaaaggaggttaaaaccgaattgactagagacgttttaaccgatcccattgtcggagtcaacaacctcagaggctacggaacaaccttctctaacatagaaaactacattcgtaaaccacatctattcgactatctgcacagaattcagtttcacacgcggttccaaccaggatactatggaaatgactctttcaactattggtccggtaattatgtttcaactagacccagcataggatctaatgacatcatcacctctccattctacggaaacaagtcctccgagcctgtgcaaaacttggagtttaatggagagaaagtctatagagccgtggccaataccaatcttgccgtctggccgtccgctgtgtactcaggtgttaccaaagtggaattcagccaatacaatgatcagacagatgaagcaagtactcaaacttacgactcaaagaggaatgttggcgcggtcagctgggattctatcgatcaactccctccagaaaccaccgatgaacctctagagaagggttatagccatcaactcaattacgtaatgtgctttctcatgcagggtagtagaggtaccatcccagtgttaacttggactcacaagagtgtagacttcttcaacatgattgattcgaaaaagattactcaacttccgttggtaaaggcctacaagttacaatctggtgcttccgttgtcgcaggtcctaggtttacaggaggagatatcattcaatgcactgagaatgggtccgcggcaactatctacgttacacctgatgtgtcgtactctcaaaagtatcgtgctagaattcattatgcttctacctctcagataacattcacactaagcttggacggggctccattcaaccaatactacttcgataagaccatcaacaaaggagacacactcacgtataattcattcaacttagccagcttcagcactccattcgaattgtcagggaacaacttgcagataggcgtcacaggattgagtgctggtgacaaggtttacatcgacaagattgagttcattccagtgaaccttaggtccccaggaaccgagcttgagttcatcgacatctag

SEQ ID NO: 13 - DNA, 216 bp, Artificial Sequence
  Synthetic sequence based on Bacillus thuringiensis
atgaagaagaacggctacgccgtggacagcagcggcaaggccccagagtgcctcctcagcaactactgcaacaaccagtgcaccaaggtgcactacgccgacaagggctactgctgcctcctcagctgctactgcttcggcctcaacgacgacaagaaggtgcttgagatcagcgacaccaggaagagctactgcgacaccaccatcatcaactaa

SEQ ID NO: 14 - DNA, 192 bp, Artificial Sequence
  Synthetic sequence based on Bacillus thuringiensis
atggccgacggctacgtgaagggcaaaagcggctgcaaaatcagctgcttcctggacaacgacctgtgcaacgccgactgcaaatactacggcggcaaactgaacagctggtgcatcccagacaaaagcggctactgctggtgcccaaacaaaggctggaacagcatcaaaagcgagaccaacacctgctaa

SEQ ID NO: 15 - DNA, 1905 bp, Artificial Sequence
  Synthetic sequence based on Bacillus thuringiensis
atgggacacaacgtccttaataacgaacgtaacacagtctgtgaagcacacaacgtggccgttcgcgaccctttctcattcgagcacaaatcactaaacactgttgagaaggagtggactgagtggaagagaaacgaccattcgctatatgttgacccgatcgttggaaccgtggcttcattcttgctcaagaaggtgggttctctcgttggtaagaggattctctcggaactaaggagcttgatcttcccctccggtagcacaaatctcatgcaggatatactccgtgagaccgagaagttcctgaaccagcgactgaacacggacaccttggcacgagttaatgctgaattgacaggtctacaggcaaatgtcgaggagttcaatcgccaagttgacaacttcctaaatcccaatcgtaacgccttgcctttgtctattacgtcgtccgtcaacacgatgcagcagctattcttgaaccggttaccacaattccagatccaaggctaccaattgttgttactcccgttattcgcccaagctgctaatcttcacctgagcttcatcagggatgtcatcctaaatgcagacgagtggggcatatcggcagctacactacgtacttatcgaaaccatctgaggaactacacgcgtgactactcaaactactgtatcaacacctatcagactgccttccgtggcctgaatacaaggctccacgatatgttggagtttcggacatacatgttcctgaacgtgttcgagtatgtctccatctggtcgcttttcaagtaccagtcattgctggtctcgtcaggtgctaacctatacgcaagtggaagtggacctcagcaaacccaatcgttcacgagtcaagactggccattcctgtatagcttgttccaggtcaactccaactacgtgctgaacggcttctcaggtgctcgattgtcccagactttcccaaacatcgttggacttccaggaacaactacgactcatgccttgctggctgcacgagtcaactactctggtggagtttcaagtggcgatattggagcttcgccgttcaaccagaacttcagttgcagcacattcctgcctcctttgcttacgccattcgttagatcctggctcgacagtggaagtgatcgagggggagttaatactgtgaccaactggcagacagagagtttcgagacaacactcggtctacgatcaggagcattcacagcaagaggaaacagcaactacttcccagactacttcatccgaaacatctctggagtacctctagtcgttaggaacgaagaccttcgtcgtcctctgcactacaatcagatcaggaacattgagtcaccttcaggtacacctggtggagcacgagcatacatggtctcagttcacaaccgtaagaacaacatctacgcagttcatgagaacggaacgatgatccacttggctccagaggactacaccggatttacaatcagtcctatccacgccactcaggtgaacaaccagactcgaacgttcatcagtgagaagtttggaaaccaaggcgattctctgaggtttgagcagagtaataccacggcaaggtacactctcaggggtaatggaaactcttacaacctatacttgcgtgtctccagcataggcaattcaactatcagggttaccatcaacggtcgagtgtacacagctacaaacgtcaataccaccactaacaacgatggtgtaaacgacaatggtgctcgcttcagcgacatcaacatcggaaacgtagtcgcgagcgacaacagcaacgtacctctggacattaacgttacgttgaactcaggcacacagttcgatttgatgaacatcatgtttgttccgacaaacctaccaccattgtattag

SEQ ID NO: 16 - DNA, 1179 bp, Rhyzopus oryzae
atggtttcattcatttccatttctcaaggtgttagtctttgtcttcttgtctcttccatgatgctcggttcatctgctgttcctgtttctggtaaatctggatcttccactaccgccgtctctgcatctgacaattctgccctccctcctctcatttccagccgttgtgctcctccttctaacaagggaagtaaaagcgatcttcaagctgaaccttactacatgcaaaagaatacagaatggtatgagtcccatggtggcaacctgacatccatcggaaagcgagatgacaatttggttggtggcatgactttggatttacctagcgatgctcctcctatcagcctctctggatctaccaacagcgcctctgatggtggtaaggttgttgctgctactactgctcaaattcaagagttcaccaagtatgctggtatcgctgccactgcctactgtcgttctgttgtccctggtaacaagtgggactgtgtccaatgtcaaaagtgggttcctgatggcaagatcatcactacctttacctccttgctttccgacacaaatggttacgtcttgagaagtgataaacaaaagaccatttatcttgttttccgtggtaccaactccttcagaagtgccatcactgatattgtcttcaacttttccgactacaagcctgtcaagggcgccaaggttcatgctggtttcctttcctcttatgagcaagttgtcaatgactatttccctgtcgtccaagaacaactgaccgccaaccctacttacaaggtcatcgtcaccggtcactcactcggtggtgcacaagctttgcttgccggtatggatctctaccaacgtgaaccaagactgtctcccaagaatttgagcatcttcactgttggtggtcctcgtgttggtaaccccacctttgcttactatgttgaatctaccggtattcctttccaacgtaccgttcacaagagagatatcgttcctcacgttcctcctcaatccttcggattccttcatcccggtgttgaatcttggattaagtctggtacctccaacgttcaaatctgtacttctgaaattgaaaccaaggattgcagtaactctatcgttcctttcacctctctccttgatcacttgagttactttgatatcaacgaaggaagctgtttgtaa

(19) United States
(12) Patent Application Publication
(10) Pub. No.: US 2006/0236424 A1
(43) Pub. Date: Oct. 19, 2006

(54) Methods and compositions for designing nucleic acid molecules for polypeptide expression in plants using plant virus codon-bias

(76) Inventors: Andre R. Abad, West Des Moines, IA (US); Ronald D. Flannagan, Grimes, IA (US); Rafael Herrmann, Wilmington, DE (US); Albert L. Lu, Newark, DE (US); Billy F. McCutchen, Clive, IA (US); Carl R. Simmons, Des Moines, IA (US)

Correspondence Address: MORGAN & FINNEGAN, L.L.P., 3 World Financial Center, New York, NY, 10281-2101, US

(21) Appl. No.: 11/399,028

(22) Filed: Apr. 5, 2006

Related U.S. Application Data
(60) Provisional application No. 60/668,734, filed on Apr. 5, 2005.

(51) Int. Cl. A01H 5/00; A01H 1/00; C12N 15/82; C12N 5/04

(52) U.S. Cl. 800/280; 800/320.1; 800/312; 435/468; 435/419

(57) ABSTRACT

The present invention relates to methods of designing nucleic acid molecules for improved expression of the encoded polypeptides in plants. In such methods, codon usage frequencies are biased towards codon usage frequencies of a plant virus, group of plant viruses, or a subset of nucleic acid molecules therefrom. In preferred embodiments, the encoded polypeptide affects the phenotype of the plant. The invention also pertains to nucleic acid molecules encoding insecticidal polypeptides wherein the nucleic acid molecules have been designed to have plant virus codon-biased. The invention also pertains to transgenic plants and progeny thereof with increased expression of insecticidal polypeptides for improved resistance to insects and other pests that are detrimental to plants of agricultural value.

This application claims benefit of U.S. provisional application No. 60/668,734, filed Apr. 5, 2005, which is incorporated herein by reference in its entirety.

### 1. FIELD OF THE INVENTION

The present invention relates to methods of designing nucleic acid molecules for improved expression of the encoded polypeptides in plants. In such methods, codon usage frequencies are biased towards codon usage frequencies of plant viruses. In preferred embodiments, the encoded polypeptide affects the phenotype of the plant. In a specific embodiment, the encoded polypeptide is an insecticidal polypeptide.

### 2. BACKGROUND OF THE INVENTION

A high level of transgenic polypeptide expression is often difficult to achieve in plants, particularly when the transgene encoding a foreign polypeptide is derived from an organism that is evolutionarily distant from plants. This has been a major hindrance to the successful exploitation of insecticidal polypeptide genes derived from prokaryotes. A critical reason for low levels of transgenic polypeptide expression is the significant difference in codon usage often observed between highly divergent species, e.g., plants and prokaryotes, commonly referred to as codon bias. Codon bias often correlates with the efficiency of translation of messenger RNA (mRNA), which is in turn believed to be dependent on, inter alia, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal expression in plants, based on these translational factors.

In general there have been two main approaches to codon biasing synthetic gene sequences for expression in plants: codon usage frequency biasing and preferred codon biasing. Codon usage frequency biasing refers to selecting codons for a nucleic acid molecule encoding the amino acid sequence of a polypeptide to be expressed, such that the codon usage frequencies for one or more types of amino acid encoded in a synthetic gene, resemble the codon usage frequencies of the polypeptide expression host (e.g. a plant). Preferred codon biasing consists of selecting codons for a nucleic acid molecule that encodes the amino acid sequence of a polypeptide to be expressed, such that one or more codons for one or more types of amino acid in a synthetic gene are the single codons that most frequently encode a type of amino acid in a polypeptide expression host (e.g. a plant). These approaches to improving transgene expression in plants, particularly with respect to the expression of insecticidal Bacillus thuringiensis CRY polypeptides, have been used in a number of cases.

Adang et al., U.S. Pat. No. 5,380,831 refers to a synthetic variant of a native Bacillus thuringiensis tenebrionsis (Btt) Cry insecticidal polypeptide gene, in which codon usage frequencies were adjusted to be close to those used in dicotyledonous plant genes. Adang et al. also indicates that the same approach may be used to generate a synthetic Cry gene adapted to expression in monocotyledonous plants, by using the codon usage frequencies of a monocotyledonous plant. Adang et al. disclose that the synthetic gene is designed by changing individual codons from the native Cry gene so that the overall codon usage frequency resembles that of a dicotyledonous plant gene.

Fischhoff et al. U.S. Pat. No. 5,500,365, refers to plant genes encoding the Cry insecticidal polypeptide from Bacillus thuringiensis. The percentages listed are based on dicotyledonous plant gene codon usage frequencies. Fischoff et al. state that in general, codons should preferably be selected so that the GC content of the synthetic gene is about 50%.

Barton et al., U.S. Pat. No. 5,177,308, is directed to the expression of insecticidal toxins in plants. A synthetic AaIT insecticidal polypeptide gene derived from a native scorpion gene is described, in which the most preferred codon is stated to be used for each amino acid.

Koziel et al., U.S. Pat. No. 6,121,014, is directed towards optimizing expression of polypeptides in plants and particularly insecticidal polypeptides from Bacillus thuringiensis. Koziel et al. indicate that the design of synthetic genes optimized for expression in monocotyledonous or dicototyledonous plants is to be based on changing a sufficient number of codons from a native sequence to the preferred codons of the host plant.

In general, increasing the translational efficiency of transgenes in plants has been attempted by generating synthetic genes that use either the preferred codons of a plant host or the codon usage frequency of the plant host. It should be noted, however, that much of the apparent maize codon bias may be due to factors unrelated to translational efficiency per se, such as plant genomic methylation selection pressure and mutation rates of methylated versus nonmethylated sites. Thus, there remains an unmet need in the art for alternative approaches for changing codon usage frequencies to increase plant transgene expression.

### 3. SUMMARY OF THE INVENTION

The present invention relates to methods of designing nucleic acid molecules for improved expression of the encoded polypeptides in plants. Accordingly, at least one codon of the nucleic acid molecule to be expressed is altered to a codon that has a usage frequency in a plant virus that is greater than that of the unaltered codon. Preferably, the nucleic acid molecules of this invention will improve expression of the encoded polypeptide as compared to a polypeptides encoded by a nucleic acid molecule that has not been altered.

In one embodiment, the altered codon has been altered to a codon that has a usage frequency in a plant virus that is greater than 0.09. In another embodiment, the altered codon has been altered to a codon that has a usage frequency in a plant virus that is equal to or greater than the median codon usage frequency for that particular amino acid encoded by the altered codon. Such a median codon usage frequency is the median of the codon usage frequencies in the plant virus for all codons encoding a particular amino acid.

In preferred embodiments, the encoded polypeptide affects the phenotype of the plant. In a specific embodiment, the encoded polypeptide is an insecticidal polypeptide including, but not limited to, the 437N and Cry polypeptides from Bacillus thuringiensis and insecticidal lipase polypeptide form Rhyzopus oryzae.

Also encompassed by the present invention are vectors, host cells, transgenic plants and progeny thereof comprising nucleic acid molecules made according to the methods of the invention. The invention further relates to plant propagating material of a transformed plant including, but not limited to, seeds, tubers, corms, bulbs, leaves, and cuttings of roots and shoots.

### 4. BRIEF DESCRIPTION OF THE DRAWINGS

FIGS. 1A-1B show the results of a leaf disk assay against the European corn borer. Leaf disks of calli transformed with codon optimized Bacillus thuringiensis insecticidal polypeptide 473N were incubated with a neonate European corn borer insect for 48 hrs. Control leaf discs from non-transgenic plants were included for comparison of leaf consumption. (A) The leaf disc was totally consumed in control wells leaving only the filter paper disc on which the leaf disk was placed (see row 1). Leaf disks transformed with codon optimized 473N were consumed very little (see row 2). (B) Additional transformation events with codon optimized 473N showed little leaf consumption.

FIG. 2 shows an immunoblot analysis of plants transformed with codon optimized Bacillus thuringiensis insecticidal polypeptide 473N. Transgenic plant polypeptide extractions were subjected to immunoblot analysis using an anti-473N antibody. Recombinant purified 473N is shown in lane 1. A control non transgenic plant sample shows non-437N cross reactive bands in common with transgenic samples (lane 2). The presence of a band corresponding to 437N was present in leaf samples from events that demonstrated efficacy in the leaf disc assay (lanes 2, 3, 4, and 7).

FIG. 3 shows an immunoblot analysis of plants transformed with codon optimized insecticidal lipase from Rhyzopus oryzae. Transgenic plant polypeptide extractions from (A) leaf and (B) root tissue were subjected to immunoblot analysis using an anti-Rolipase antibody. Purified recombinant Rolipase precursor protein (ROL˜42 kD) was included in the immunoblot analysis as a positive control. The presence of a band corresponding to mature Rolipase (˜31 kD) was seen in plants that were positive in the root trainer assay (lanes 1-6).

### 5. DETAILED DESCRIPTION OF THE INVENTION

Biological systems exhibit characteristic frequencies in the usage of particular codons (i.e. codon usage frequencies) to specify a given type of amino acid. Such codon frequencies can differ greatly from species to species, a phenomenon known as “codon bias”. Species differences in codon bias are possible due to the degeneracy of the genetic code and are well documented, in the form of codon usage frequency tables. The codon bias of a particular nucleic acid molecule will determine, to a large degree, the efficiency with which the encoded polypeptide is expressed in a particular type of cell.

The effect of codon bias on expression efficiency is a particularly important consideration for transgene expression. An mRNA sequence comprising many codons that are not used frequently in a species that is to be the expression host is unlikely to be translated efficiently. Conversely, an mRNA sequence that consists of codons that are frequently used by a host organism is likely to be translated with high efficiency.

The present invention relates to methods of designing nucleic acid molecules for improved expression of the encoded polypeptides in plants by constructing nucleic acid molecules that are codon-biased towards codons that are used frequently in nucleic acid molecule coding sequences of plant viruses. The codon bias of plant viruses known to exploit plant host translational machinery with high efficiency is more likely to be a reflection of plant host translational preferences than the codon bias of the native plant host genomic sequences. Accordingly, at least one codon of the nucleic acid molecule to be expressed is altered to a codon that has a usage frequency in a plant virus, group of plant viruses, or subset of nucleic acid molecules therefrom that is greater than that of the unaltered codon. Preferably, the nucleic acid molecules of this invention will improve expression of the encoded polypeptide as compared to a polypeptide encoded by a nucleic acid molecule that has not been altered.

The methods of the present invention comprise generating codon usage frequency tables from a plant virus, group of plant viruses, or a subset of nucleic acid molecules therefrom of interest to determine codons with high usage frequencies in plant viruses. Such high usage frequency codons can be substituted for codons with low usage frequencies that are present in nucleic acid molecules to be expressed in plants. The codons with the higher usage frequencies that used in the substitutions are termed “altered codons”. Nucleic acid molecules and their encoded polypeptides that have at least one altered codon are said to be “codon optimized”. There is no requirement that all or majority codons must be altered codons for a nucleic acid molecule or polypeptide to be a codon optimized molecule.

### 5.1 Determining Codon Usage Frequencies

In order to alter a nucleic acid molecule such that the altered codons are those with a higher usage frequency in a plant virus, one must first determine codon usage frequencies for the plant virus. In one embodiment, the codon usage frequency is based on all of the polypeptides encoded by the virus nucleic acid molecules. In another embodiment, the codon usage frequency is based on a subset of the polypeptides encoded by the virus nucleic acid molecules. In another embodiment, the codon usage frequency is based on the subset of the polypeptides encoded by the virus nucleic acid molecules that are similar in function (e.g., the coat polypeptides, the transcriptional or translational machinery polypeptides, the envelope polypeptides, etc.). The codon usage frequency can be based on one plant virus or multiple plant viruses. In embodiments where multiple plant viruses are used to calculate codon usage frequencies, the viruses preferably infect the same type of plant (e.g., monocot, dicot, maize, soybean, etc.).

Codon usage frequency is calculated for a nucleic acid molecule coding sequence according to the following method. First, the total number of all codons encoding a particular type of amino acid (or a stop codon) is determined by counting the occurrences over one or more nucleic acid molecule coding sequences. Second, the total number of occurrences for each codon encoding a particular type of amino acid (or stop codon) is determined for the same nucleic acid molecule coding sequences. Third, a codon usage frequency for each codon is determined by dividing the total number of occurrences of that codon by the total number of occurrences of codons encoding the same type of amino acid as that codon.

Tables disclosed in Sections 5.1.1, 5.1.2, and 5.2 may be used to select the codons to be used as altered codons. Alternatively, the skilled artisan may generate distinct tables with viruses of interest using the methods described herein.

### 5.1.1 Monocotyledonous Plant Virus Codon-Biased

In some embodiments, a plant virus or viruses that infect monocotyledonous plants are used to generate codon usage frequencies. As a non-limiting example, monocotyledonous plant virus codon usage frequencies were determined for 173 nucleic acid molecule coding sequences from monocotyledonous plant viruses (listed in Table 1). The sequences used comprise, as Table 2 indicates, the codon usage frequencies determined from the nucleic acid molecule coding sequences of the monocotyledonous viruses listed in Table 1. The monocotyledonous plant virus codon usage frequencies listed in Table 2 can be used to guide the selection of codons for design of a plant virus codon-biased nucleic acid molecule coding sequence encoding a polypeptide to be expressed in a plant. Viral sequences can be obtained from any source, e.g., Genbank and NCBI taxonomy database. If expression of the polypeptide encoded by the nucleic acid molecule comprising altered codons is desired in a moncotyledonous plant, preferably plant viruses that infect monocots are used to generate the codon usage frequencies (as, e.g., in Table 2).

TABLE 1
Monocotyledonous plant viruses and number of sequences
from each used for codon usage frequency calculation.

| Monocot Plant Virus (173 Sequences) | Number of Sequences |
|---|---|
| Barley mild mosaic virus | 5 |
| Barley yellow dwarf virus | 5 |
| Barley yellow dwarf virus-GAV | 2 |
| Barley yellow dwarf virus-PAV | 3 |
| Barley yellow dwarf virus (isolate P-PAV) | 1 |
| Barley yellow dwarf virus-PAS | 1 |
| Cereal yellow dwarf virus-RPV | 2 |
| Chloris striate mosaic virus | 2 |
| Maize chlorotic mottle virus | 2 |
| Maize dwarf mosaic virus | 2 |
| Maize rayado fino virus | 1 |
| Maize rough dwarf virus | 2 |
| Maize streak virus | 10 |
| Maize stripe virus | 7 |
| Mal de Rio Cuarto virus | 6 |
| Oat necrotic mottle virus | 1 |
| Oat sterile dwarf virus | 2 |
| Panicum streak virus | 3 |
| Rice black streaked dwarf virus | 15 |
| Rice black streaked dwarf virus | 3 |
| Rice dwarf virus | 13 |
| Rice dwarf virus | 12 |
| Rice gall dwarf virus | 6 |
| Rice hoja blanca virus | 6 |
| Rice ragged stunt virus | 10 |
| Rice stripe virus | 7 |
| Rice tungro bacilliform virus | 8 |
| Rice tungro bacilliform virus | 5 |
| Rice tungro spherical virus | 2 |
| Rice yellow mottle virus | 7 |
| Sugarcane bacilliform virus | 6 |
| Sugarcane streak Egypt virus | 3 |
| Sugarcane streak Reunion virus | 3 |
| Sugarcane streak virus | 3 |
| Wheat dwarf virus | 1 |
| Wheat rosette stunt virus | 2 |
| Wheat streak mosaic virus | 2 |
| Wheat yellow mosaic virus | 2 |

TABLE 2

Monocotyledonous plant virus codon usage
frequencies.

| | | |
|---|---|---|
| | | Monocot |
| | | Plant Virus |
| | | Codon |
| Amino Acid | Codon | Freq. |

| | | |
|---|---|---|
| Ala | GCA | 0.31 |
| | GCC | 0.21 |
| | GCG | 0.14 |
| | GCT | 0.34 |
| Arg | AGA | 0.32 |
| | AGG | 0.17 |
| | CGA | 0.14 |
| | CGC | 0.14 |
| | CGG | 0.09 |
| | CGT | 0.16 |
| Asn | AAC | 0.42 |
| | AAT | 0.58 |
| Asp | GAC | 0.38 |
| | GAT | 0.62 |
| Cys | TGC | 0.44 |
| | TGT | 0.56 |
| Gln | CAA | 0.58 |
| | CAG | 0.42 |
| Glu | GAA | 0.60 |
| | GAG | 0.40 |
| Gly | GGA | 0.37 |
| | GGC | 0.20 |
| | GGG | 0.14 |
| | GGT | 0.28 |
| His | CAC | 0.43 |
| | CAT | 0.57 |
| Ile | ATA | 0.30 |
| | ATC | 0.29 |
| | ATT | 0.41 |
| Leu | CTA | 0.13 |
| | CTC | 0.14 |
| | CTG | 0.13 |
| | CTT | 0.18 |
| | TTA | 0.21 |
| | TTG | 0.21 |
| Lys | AAA | 0.53 |
| | AAG | 0.47 |
| Met | ATG | 1.00 |
| Phe | TTC | 0.46 |
| | TTT | 0.54 |
| Pro | CCA | 0.38 |
| | CCC | 0.17 |
| | CCG | 0.14 |
| | CCT | 0.31 |
| STOP | TAA | 0.34 |
| | TAG | 0.25 |
| | TGA | 0.41 |
| Ser | AGC | 0.13 |
| | AGT | 0.18 |
| | TCA | 0.24 |
| | TCC | 0.14 |
| | TCG | 0.10 |
| | TCT | 0.21 |
| Thr | ACA | 0.30 |
| | ACC | 0.20 |
| | ACG | 0.16 |
| | ACT | 0.34 |
| Trp | TGG | 1.00 |
| Tyr | TAC | 0.43 |
| | TAT | 0.57 |
| Val | GTA | 0.19 |
| | GTC | 0.21 |
| | GTG | 0.25 |
| | GTT | 0.36 |

In specific embodiments, codon usage frequencies are based on a monocot plant virus or viruses that infect a specific monocot plant type (e.g., maize). In one specific embodiment, codon usage frequencies were calculated using nucleic acid molecule coding sequences from maize viruses, wherein the nucleic acid molecules have the following accession numbers: CAA68570, CAA68567, CAA68566, CAA68568, CAA68569, CAA12314, CAA12315, CAA12316, CAA12317, CAA12318, CAA12319, CAA12320, NP_{—}115454, NP_{—}115455, AAB22541, AAB22542, AAB26111, AAP80680, AAP80681, AAA46635, AAA46636, AAA46637, NP_{—}569138, NP_{—}619717, NP_{—}619718, NP_{—}619719, NP_{—}619720, NP_{—}619721, NP_{—}619722, AAB50194, AAB50195, CAA39227, and CAA39228 (Table 3). In another specific embodiment, codon usage frequencies are calculated for a subset of the nucleic acid molecules from a maize specific virus or viruses. Nucleic acid molecules encoding coat polypeptides for maize-specific viruses (having accession numbers CAA68566, AAP80681, AAA46637, and NP_{—}619722) were used to generate Table 4. If expression of the polypeptide encoded by the nucleic acid molecule comprising altered codons is desired in maize, preferably plant viruses that infect maize are used to generate the codon usage frequencies (as, e.g., in Tables 3 and 4).

TABLE 3
Maize-specific virus codon usage frequencies.

| | | |
|---|---|---|
| | | Maize |
| | | Viral |
| | | Codon |
| Amino Acid | Codon | Freq. |

| | | |
|---|---|---|
| Ala | GCA | 0.31 |
| | GCC | 0.3 |
| | GCG | 0.11 |
| | GCT | 0.28 |
| Arg | AGA | 0.27 |
| | AGG | 0.17 |
| | CGA | 0.12 |
| | CGC | 0.19 |
| | CGG | 0.12 |
| | CGT | 0.13 |
| Asn | AAC | 0.44 |
| | AAT | 0.56 |
| Asp | GAC | 0.41 |
| | GAT | 0.59 |
| Cys | TGC | 0.42 |
| | TGT | 0.58 |
| Gln | CAA | 0.5 |
| | CAG | 0.5 |
| Gln | CAA | 0.52 |
| | CAG | 0.48 |
| Gly | GGA | 0.36 |
| | GGC | 0.23 |
| | GGG | 0.17 |
| | GGT | 0.24 |
| His | CAC | 0.45 |
| | CAT | 0.55 |
| Ile | ATA | 0.27 |
| | ATC | 0.3 |
| | ATT | 0.43 |
| Leu | CTA | 0.12 |
| | CTC | 0.22 |
| | CTG | 0.16 |
| | CTT | 0.19 |
| | TTA | 0.14 |
| | TTG | 0.18 |
| Lys | AAA | 0.49 |
| | AAG | 0.51 |
| Met | ATG | 1 |
| Phe | TTC | 0.56 |
| | TTT | 0.44 |
| Pro | CCA | 0.31 |
| | CCC | 0.20 |
| | CCG | 0.17 |
| | CCT | 0.32 |
| STOP | TAA | 0.33 |
| | TAG | 0.42 |
| | TGA | 0.24 |
| Ser | AGC | 0.12 |
| | AGT | 0.12 |
| | TCA | 0.22 |
| | TCC | 0.21 |
| | TCG | 0.10 |
| | TCT | 0.22 |
| Thr | ACA | 0.32 |
| | ACC | 0.26 |
| | ACG | 0.13 |
| | ACT | 0.29 |
| Trp | TGG | 1.00 |
| Tyr | TAC | 0.46 |
| | TAT | 0.54 |
| Val | GTA | 0.16 |
| | GTC | 0.25 |
| | GTG | 0.26 |
| | GTT | 0.33 |

TABLE 4

Maize-specific virus capsid/coat polypeptide codon
usage frequencies

| | | |
|---|---|---|
| | | Maize |
| | | Viral Coat |
| | | Codon |
| Amino Acid | Codon | Freq. |

| | | |
|---|---|---|
| Ala | GCA | 0.38 |
| | GCC | 0.22 |
| | GCG | 0.14 |
| | GCT | 0.26 |
| Arg | AGA | 0.3 |
| | AGG | 0.18 |
| | CGA | 0.18 |
| | CGC | 0.16 |
| | CGG | 0.11 |
| | CGT | 0.07 |
| Asn | AAC | 0.53 |
| | AAT | 0.47 |
| Asp | GAC | 0.45 |
| | GAT | 0.55 |
| Cys | TGC | 0.53 |
| | TGT | 0.47 |
| Gln | CAA | 0.52 |
| | CAG | 0.48 |
| Gln | GAA | 0.44 |
| | GAG | 0.56 |
| Gly | GGA | 0.42 |
| | GGC | 0.18 |
| | GGG | 0.23 |
| | GGT | 0.18 |
| His | CAC | 0.35 |
| | CAT | 0.65 |
| Ile | ATA | 0.24 |
| | ATC | 0.36 |
| | ATT | 0.40 |
| Leu | CTA | 0.12 |
| | CTC | 0.18 |
| | CTG | 0.25 |
| | CTT | 0.12 |
| | TTA | 0.10 |
| | TTG | 0.23 |
| Lys | AAA | 0.48 |
| | AAG | 0.52 |
| Met | ATG | 1.00 |
| Phe | TTC | 0.57 |
| | TTT | 0.43 |
| Pro | CCA | 0.32 |
| | CCC | 0.24 |
| | CCG | 0.12 |
| | CCT | 032 |
| STOP | TAA | 0.50 |
| | TAG | 0 |
| | TGA | 0.50 |
| Ser | AGC | 0.19 |
| | AGT | 0.13 |
| | TCA | 0.21 |
| | TCC | 0.26 |
| | TCG | 0.06 |
| | TCT | 0.15 |
| Thr | ACA | 0.36 |
| | ACC | 0.27 |
| | ACG | 0.06 |
| | ACT | 0.31 |
| Trp | TGG | 1 |
| Tyr | TAC | 0.41 |
| | TAT | 0.59 |
| Val | GTA | 0.15 |
| | GTC | 0.26 |
| | GTG | 0.36 |
| | GTT | 0.23 |

### 5.1.2 Dicotyledonous Plant Virus Codon-Biased

In some embodiments, a plant virus or viruses that infect dicotyledonous plants are used to generate codon usage frequencies. As a non-limiting example, dicotyledonous plant virus codon usage frequencies were determined for 321 nucleic acid molecule coding sequences from dicotyledonous plant viruses (listed in Table 5). Table 6 indicates the codon usage frequencies determined from the nucleic acid molecule coding sequences of the dicotyledonous viruses listed in Table 5. The dicotyledonous plant virus codon usage frequencies listed in Table 6 can be used to guide the selection of codons for design of a plant virus codon-biased nucleic acid molecule coding sequence encoding a polypeptide to be expressed in a plant. If expression of the polypeptide encoded by the nucleic acid molecule comprising altered codons is desired in a dicotyledonous plant, preferably plant viruses that infect dicots are used to generate the codon usage frequencies (as, e.g., in Table 6).

In one specific embodiment, codon usage frequencies are calculated for a subset of the nucleic acid molecules from a dicot plant virus or viruses. Nucleic acid molecules encoding coat polypeptides from a number of different dicot plant viruses (listed in Table 7) were used to generate Table 8.

In another specific embodiments, codon usage frequencies are based on a dicot plant virus or viruses that infect a specific dicot plant type (e.g., soybean). If expression of the polypeptide encoded by the nucleic acid molecule comprising altered codons is desired in a particular type of plant (e.g., soybean), preferably plant viruses that infect that type of plant (e.g., soybean specific viruses) are used to generate the codon usage frequencies.

TABLE 5
Dicotyledonous plant viruses and number of sequences from each used for codon
usage frequency calculation

| Dicot Plant Virus (321 sequences) | # | (Continued) | # |
|---|---|---|---|
| African cassava mosaic virus | 4 | Papaya ringspot virus | 1 |
| Artichoke mottled crinkle virus | 3 | Papaya ringspot virus W | 1 |
| Bean calico mosaic virus | 4 | Parsnip yellow fleck virus | 1 |
| Bean common mosaic necrosis virus | 1 | Peanut chlorotic streak virus | 4 |
| Bean common mosaic virus | 2 | Pepper golden mosaic virus | 2 |
| Bean dwarf mosaic virus | 5 | Pepper golden mosaic virus-[CR] | 3 |
| Bean golden mosaic virus | 5 | Pepper yellow vein Mali virus | 3 |
| Bean golden yellow mosaic virus | 4 | Potato aucuba mosaic virus | 5 |
| Bean leafroll virus | 2 | Potato leafroll virus | 2 |
| Bean pod mottle virus | 1 | Potato virus S | 10 |
| Beet curly top virus | 2 | Potato yellow mosaic virus | 3 |
| Beet mild curly top virus | 2 | Potato yellow mosaic virus- | 5 |
| | | [Guadeloupe] | |
| Beet severe curly top virus | 2 | Prune dwarf virus | 7 |
| Broadhaven virus | 2 | Red clover mottle virus | 2 |
| Carnation etched ring virus | 6 | Red clover necrotic mosaic | 4 |
| | | virus | |
| Carnation ringspot virus | 4 | Sesbania mosaic virus | 2 |
| Cassava vein mosaic virus | 5 | South African cassava mosaic | 3 |
| | | virus | |
| Cauliflower mosaic virus | 9 | Southern cowpea mosaic | 2 |
| | | virus | |
| Clover yellow vein virus | 1 | Soybean chlorotic mottle | 8 |
| | | virus | |
| Commelina yellow mottle virus | 3 | Soybean dwarf virus | 5 |
| Cowpea aphid-borne mosaic virus | 1 | Soybean mosaic virus | 2 |
| Cowpea mosaic virus | 2 | Soybean yellow mosaic virus | 3 |
| Cucumber necrosis virus | 3 | Squash leaf curl virus | 8 |
| Cucurbit leaf curl virus-[Arizona] | 5 | Squash leaf curl virus- | 3 |
| | | Vietnam | |
| Dianthovirus RVX1 | 4 | Squash mild leaf curl virus | 5 |
| Digitaria streak virus | 3 | Squash mosaic virus | 3 |
| Dioscorea alata bacilliform virus | 4 | Strawberry latent ringspot | 1 |
| | | virus | |
| East African cassava mosaic Cameroon | 4 | Strawberry latent ringspot | 1 |
| virus | | virus satellite RNA | |
| East African cassava mosaic virus | 3 | Strawberry vein banding | 6 |
| | | virus | |
| Figwort mosaic virus | 6 | Sweet clover necrotic mosaic | 3 |
| | | virus | |
| Fiji disease virus | 8 | Tobacco vein mottling virus | 1 |
| Indian cassava mosaic virus-[Maharashtra] | 2 | Tobacco yellow dwarf virus | 3 |
| Kalanchoe top-spotting virus | 3 | Tomato golden mosaic virus | 4 |
| Kennedya yellow mosaic virus | 4 | Tomato golden mosaic virus- | 1 |
| | | Common | 1 |
| Lettuce infectious yellows virus | 6 | Tomato leaf curl Mali virus | 3 |
| Lettuce mosaic virus | 1 | Tomato mottle Taino virus | 4 |
| Macroptilium mosaic virus | 1 | Tomato mottle virus | 5 |
| Mirabilis mosaic virus | 7 | Tomato spotted wilt virus | 5 |
| Miscanthus streak virus | 4 | | |
| Mungbean yellow mosaic India virus- | 2 | | |
| [SoybeanTN] | | | |
| Mungbean yellow mosaic virus- | 3 | | |
| Soybean[Madurai] | | | |
| Tomato yellow leaf curl Kanchanaburi | 3 | | |
| virus-[Thailand Kan2] | | | |
| Tomato yellow leaf curl Mali virus | 2 | | |
| Tomato yellow leaf curl Sardinia virus | 2 | | |
| Tomato yellow leaf curl Sardinia virus- | 2 | | |
| [Spain1] | | | |
| Tomato yellow leaf curl Thailand virus | 2 | | |
| Tomato yellow leaf curl Thailand virus-[1] | 1 | | |
| Tomato yellow leaf curl Thailand virus | 7 | | |
| Turnip crinkle virus | 4 | | |
| Wound tumor virus | 9 | | |
| Potato leafroll virus | 1 | | |
| Tomato golden mosaic virus | 5 | | |
| Tomato yellow leaf curl China virus | 3 | | |
| Tomato yellow leaf curl Kanchanaburi | 2 | | |
| virus-[Thailand Kan1] | | | |
| Tomato yellow leaf curl Malaga virus | 1 | | |
| Tomato yellow leaf curl China virus | 3 | | |
| Tomato yellow leaf curl Kanchanaburi | 2 | | |
| virus-[Thailand Kan1] | | | |
| Tomato yellow leaf curl Malaga virus | 1 | | |

TABLE 6

Dicotyledonous plant virus codon usage
frequencies.

| | | |
|---|---|---|
| | | Dicot Viral |
| | | Codon |
| Amino Acid | Codon | Freq. |

| | | |
|---|---|---|
| Ala | GCA | 0.33 |
| | GCC | 0.21 |
| | GCG | 0.13 |
| | GCT | 0.33 |
| Arg | AGA | 0.34 |
| | AGG | 0.23 |
| | CGA | 0.11 |
| | CGC | 0.09 |
| | CGG | 0.08 |
| | CGT | 0.15 |
| Asn | AAC | 0.41 |
| | AAT | 0.59 |
| Asp | GAC | 0.37 |
| | GAT | 0.63 |
| Cys | TGC | 0.41 |
| | TGT | 0.59 |
| Gln | CAA | 0.61 |
| | CAG | 0.40 |
| Glu | GAA | 0.61 |
| | GAG | 0.39 |
| Gly | GGA | 0.35 |
| | GGC | 0.18 |
| | GGG | 0.18 |
| | GGT | 0.29 |
| His | CAC | 0.43 |
| | CAT | 0.57 |
| Ile | ATA | 0.31 |
| | ATC | 0.28 |
| | ATT | 0.41 |
| Leu | CTA | 0.12 |
| | CTC | 0.14 |
| | CTG | 0.12 |
| | CTT | 0.19 |
| | TTA | 0.22 |
| | TTG | 0.21 |
| Lys | AAA | 0.54 |
| | AAG | 0.46 |
| Met | ATG | 1.00 |
| Phe | TTC | 0.44 |
| | TTT | 0.56 |
| Pro | CCA | 0.38 |
| | CCC | 0.18 |
| | CCG | 0.12 |
| | CCT | 0.31 |
| STOP | TAA | 0.46 |
| | TAG | 0.24 |
| | TGA | 0.30 |
| Ser | AGC | 0.14 |
| | AGT | 0.20 |
| | TCA | 0.23 |
| | TCC | 0.14 |
| | TCG | 0.08 |
| | TCT | 0.21 |
| Thr | ACA | 0.36 |
| | ACC | 0.20 |
| | ACG | 0.14 |
| | ACT | 0.31 |
| Trp | TGG | 1 |
| Tyr | TAC | 0.41 |
| | TAT | 0.59 |
| Val | GTA | 0.19 |
| | GTC | 0.21 |
| | GTG | 0.25 |
| | GTT | 0.35 |

TABLE 7
Dicotyledonous plant viruses and number of sequences of capsid/coat
polypeptide from each used for codon usage frequency calculation.

| Dicot plant virus | Number of Sequences |
|---|---|
| Artichoke mottled crinkle virus | 1 |
| Bean calico mosaic virus | 1 |
| Bean dwarf mosaic virus | 2 |
| Bean golden mosaic virus | 1 |
| Bean golden yellow mosaic virus | 1 |
| Bean leafroll virus | 1 |
| Beet curly top virus | 1 |
| Cassava vein mosaic virus | 1 |
| Cauliflower mosaic virus | 1 |
| Chloris striate mosaic virus | 1 |
| Cucumber necrosis virus | 1 |
| Cucurbit leaf curl virus-[Arizona] | 1 |
| Digitaria streak virus | 1 |
| Kennedya yellow mosaic virus | 1 |
| Lettuce infectious yellows virus | 2 |
| Macroptilium mosaic virus | 1 |
| Miscanthus streak virus | 1 |
| Pepper golden mosaic virus-[CR] | 1 |
| Pepper yellow vien Mali virus | 1 |
| Potato aucuba mosaic virus | 1 |
| Potato virus S | 2 |
| Potato yellow mosaic virus-[Guadeloupe] | 1 |
| Prune dwarf virus | 4 |
| Red clover necrotic mosaic virus | 2 |
| South African cassava mosaic virus | 1 |
| Soybean chlorotic mottle virus | 1 |
| Squash mild leaf curl virus | 1 |
| Sweet clover necrotic mosaic virus | 1 |
| Tobacco yellow dwarf virus | 1 |
| Tomato golden mosaic virus | 1 |
| Tomato leaf curl Mali virus | 1 |
| Tomato mottle Taino virus | 1 |
| Tomato mottle virus | 1 |
| Tomato spotted wilt virus | 1 |
| Tomato yellow leaf curl China virus | 1 |
| Tomato yellow leaf curl Kanchanaburi virus- | 1 |
| [Thailand Kan2] | |
| Tomato yellow leaf curl Malaga virus | 1 |
| Tomato yellow leaf curl virus | 3 |
| Turnip crinkle virus | 1 |
| Tomato golden mosaic virus | 1 |

TABLE 8

Dicotyledonous plant virus capsid/coat polypeptide
codon usage frequencies

| | | |
|---|---|---|
| | | Dicot Viral |
| | | Codon |
| Amino Acid | Codon | Freq. |

| | | |
|---|---|---|
| Ala | GCA | 0.24 |
| | GCC | 0.27 |
| | GCG | 0.15 |
| | GCT | 0.34 |
| Arg | AGA | 0.24 |
| | AGG | 0.22 |
| | CGA | 0.12 |
| | CGC | 0.10 |
| | CGG | 0.11 |
| | CGT | 0.21 |
| Asn | AAC | 0.44 |
| | AAT | 0.56 |
| Asp | GAC | 0.32 |
| | GAT | 0.68 |
| Cys | TGC | 0.25 |
| | TGT | 0.75 |
| Gln | CAA | 0.59 |
| | CAG | 0.41 |
| Glu | GAA | 0.61 |
| | GAG | 0.39 |
| Gly | GGA | 0.32 |
| | GGC | 0.2 |
| | GGG | 0.18 |
| | GGT | 0.3 |
| His | CAC | 0.35 |
| | CAT | 0.65 |
| Ile | ATA | 0.39 |
| | ATC | 0.26 |
| | ATT | 0.35 |
| Leu | CTA | 0.10 |
| | CTC | 0.13 |
| | CTG | 0.12 |
| | CTT | 0.14 |
| | TTA | 0.28 |
| | TTG | 0.23 |
| Lys | AAA | 0.54 |
| | AAG | 0.46 |
| Met | ATG | 1.00 |
| Phe | TTC | 0.44 |
| | TTT | 0.56 |
| Pro | CCA | 0.38 |
| | CCC | 0.18 |
| | CCG | 0.12 |
| | CCT | 0.31 |
| STOP | TAA | 0.46 |
| | TAG | 0.24 |
| | TGA | 0.30 |
| Ser | AGC | 0.14 |
| | AGT | 0.20 |
| | TCA | 0.23 |
| | TCC | 0.14 |
| | TCG | 0.08 |
| | TCT | 0.21 |
| Thr | ACA | 0.36 |
| | ACC | 0.20 |
| | ACG | 0.14 |
| | ACT | 0.31 |
| Trp | TGG | 1 |
| Tyr | TAC | 0.41 |
| | TAT | 0.59 |
| Val | GTA | 0.19 |
| | GTC | 0.21 |
| | GTG | 0.25 |
| | GTT | 0.35 |

### 5.2 Criteria for Selecting Codons

Once codon usage frequencies are calculated for the particular virus, group of viruses, or subset of nucleic acid molecules therefrom, codons can be chosen for use as altered codons using a variety of criteria. It should be appreciated that there are additional criteria that are not based on codon usage frequencies that can effect the final design of the nucleic acid molecule (see Section 5.3).

### 5.2.1 Increased Frequency Value Criterion

In one embodiment, any codon that has a higher usage frequency in the plant virus, viruses, or subset of nucleic acid molecules therefrom used to create the codon usage frequency table than the codon presently in the nucleic acid molecule to be designed is chosen as an altered codon. For example, if a nucleic acid molecule to be designed according to the plant virus codon biased methods of the invention has an alanine that is coded for by the GCG codon, that codon could be changed to a codon that is more frequently used in plant viruses. Using, e.g., Table 2, one skilled in the art can see that any of the other three codons for alanine (e.g., GCA, GCC, or GCT) are more frequently used in plant viruses and thus could be used as the altered codon. It should be appreciated that it is not necessary to choose the codon that is the most frequently used in plant viruses as the altered codon. Rather it is only necessary that the altered codon has a higher usage frequency in the plant virus, viruses, or nucleic acid molecules therefrom than the codon originally present in the nucleic acid molecule.

### 5.2.2 Median Value Criterion

In another embodiment, an altered codon has a codon usage frequency in the plant virus, viruses, or subset of nucleic acid molecules therefrom used to create the codon usage frequency table that is equal to or greater than the median codon usage frequency for that particular amino acid. The median value for codon usage frequencies for a given type of amino acid is determined by first, ordering all of the codons that encode that particular amino acid codon from the most frequently used to the least frequently used.

For cases where there are an odd number of codons encoding a particular type of amino acid, the median codon usage frequency is the one that has an equal number of codons used more frequently and less frequently than it. For example, isoleucine is encoded by three codons. To find the median value of codon usage frequencies, one would find the codon with an equal number of codons used more frequently and less frequently than it (in this case ATA when using the frequencies listed in Table 2). When designing a nucleic acid molecule, altered codons could be selected with usage frequencies of 0.3 or higher for isoleucine.

For cases where there are an even number of codons encoding a particular type of amino acid, the median codon usage frequency is the mean of the codon usage frequencies for the two codons that have an equal number of codons used more frequently and less frequently than them. For example, alanine is encoded by four codons. To find the median value of codon usage frequencies, one would order the codons from most frequently used to least frequently used (in this case GCT, GCA, GCC, GCG when using frequencies listed in Table 2). Because GCA and GCC have an equal number of codons used more frequently and less frequently than them, the mean of their frequency values is the median codon usage frequency (i.e., the mean of 0.31 and 0.21 is 0.26). When designing a nucleic acid molecule, altered codons could be selected with usage frequencies of 0.26 or higher for alanine.

This method biases the nucleic acid molecule coding sequence towards the use of codons that are more frequently used in plant virus nucleic acid molecule coding sequences, although not necessarily the single most frequently used codons, while minimizing the use of codons that are used less frequently (i.e., those whose codon usage frequency falls below the median codon usage frequency for a given type of amino acid).

Table 9 indicates the median values for the monocotyledonous plant virus codon usage frequencies listed in Table 2 and the codons which meet this criterion for each type of amino acid (termed selectable codons) based on their usage frequencies.

Table 10 indicates the median values for the maize-specific virus codon usage frequencies listed in Table 3 and the codons which meet this criterion for each type of amino acid based on their usage frequencies.

Table 11 indicates the median values for the maize-specific virus coat/capsid polypeptide codon usage frequencies listed in Table 4 and the codons which meet this criterion for each type of amino acid based on their usage frequencies.

Table 12 indicates the median values for dicotyledonous plant virus codon usage frequencies listed in Table 6 and the codons which meet this criterion for each type of amino acid.

Table 13 indicates the median values for the dicotyledonous virus coat/capsid polypeptide codon usage frequencies listed in Table 8 and the codons which meet this criterion for each type of amino acid based on their usage frequencies.

TABLE 9

Possible selectable codons based on median values
of monocotyledonous plant virus codon usage
frequencies

| | | | | |
|---|---|---|---|---|
| Amino | | Monocot Viral | Monocot Virus | Selectable |
| Acid | Codon | Freq. | Codon Median | Codon |

| | | | | |
|---|---|---|---|---|
| Ala | GCA | 0.31 | 0.26 | GCA |
| | GCC | 0.21 | | |
| | GCG | 0.14 | | |
| | GCT | 0.34 | | GCT |
| Arg | AGA | 0.32 | 0.15 | AGA |
| | AGG | 0.17 | | AGG |
| | CGA | 0.14 | | |
| | CGC | 0.14 | | |
| | CGG | 0.09 | | |
| | CGT | 0.16 | | CGT |
| Asn | AAC | 0.42 | 0.50 | |
| | AAT | 0.58 | AAT | |
| Asp | GAC | 0.38 | 0.50 | |
| | GAT | 0.62 | | GAT |
| Cys | TGC | 0.44 | 0.50 | |
| | TGT | 0.56 | | TGT |
| Gln | CAA | 0.58 | 0.50 | CAA |
| | CAG | 0.42 | | |
| Glu | GAA | 0.60 | 0.50 | GAA |
| | GAG | 0.40 | | |
| Gly | GGA | 0.37 | 0.24 | GGA |
| | GGC | 0.20 | | |
| | GGG | 0.14 | | |
| | GGT | 0.28 | | GGT |
| His | CAC | 0.43 | 0.50 | |
| | CAT | 0.57 | | CAT |
| Ile | ATA | 0.30 | 0.30 | ATA |
| | ATC | 0.29 | | |
| | ATT | 0.41 | | ATT |
| Leu | CTA | 0.13 | 0.16 | |
| | CTC | 0.14 | | |
| | CTG | 0.13 | | |
| | CTT | 0.18 | | CTT |
| | TTA | 0.21 | | TTA |
| | TTG | 0.21 | | TTG |
| Lys | AAA | 0.53 | 0.5 | AAA |
| | AAG | 0.47 | | |
| Met | ATG | 1.00 | 1.00 | ATG |
| Phe | TTC | 0.46 | 0.50 | |
| | TTT | 0.54 | | TTT |
| Pro | CCA | 0.38 | 0.27 | CCA |
| | CCC | 0.17 | | |
| | CCG | 0.14 | | |
| | CCT | 0.31 | | CCT |
| STOP | TAA | 0.34 | 0.34 | TAA |
| | TAG | 0.25 | | |
| | TGA | 0.41 | | TGA |
| Ser | AGC | 0.13 | 0.16 | |
| | AGT | 0.18 | | AGT |
| | TCA | 0.24 | | TCA |
| | TCC | 0.14 | | |
| | TGC | 0.10 | | |
| | TCT | 0.21 | | TCT |
| Thr | ACA | 0.30 | 0.25 | ACA |
| | ACC | 0.20 | | |
| | ACG | 0.16 | | |
| | ACT | 0.34 | | ACT |
| Trp | TGG | 1.00 | 1.00 | TGG |
| Tyr | TAC | 0.43 | 0.50 | |
| | TAT | 0.57 | | TAT |
| Val | GTA | 0.19 | 0.23 | |
| | GTC | 0.21 | | |
| | GTG | 0.25 | | GTG |
| | GTT | 0.36 | | GTT |

TABLE 10

Possible selectable codons based on median values
of maize-specific virus codon usage frequencies

| | | | | |
|---|---|---|---|---|
| Amino | | Maize Viral | Maize Viral | Selectable |
| Acid | Codon | Codon Freq. | Median | Codons |

| | | | | |
|---|---|---|---|---|
| Ala | GCA | 0.31 | 0.29 | GCA |
| | GCC | 0.3 | | GCC |
| | GCG | 0.11 | | |
| | GCT | 0.28 | | |
| Arg | AGA | 0.27 | 0.15 | AGA |
| | AGG | 0.17 | | AGG |
| | CGA | 0.12 | | |
| | CGC | 0.19 | | CGC |
| | CGG | 0.12 | | |
| | CGT | 0.13 | | |
| Asn | AAC | 0.44 | 0.5 | |
| | AAT | 0.56 | AAT | |
| Asp | GAC | 0.41 | 0.5 | |
| | GAT | 0.59 | | GAT |
| Cys | TGC | 0.42 | 0.5 | |
| | TGT | 0.58 | | TGT |
| Gln | CAA | 0.5 | 0.5 | CAA |
| | CAG | 0.5 | | CAG |
| Glu | GAA | 0.52 | 0.5 | GAA |
| | GAG | 0.48 | | |
| Gly | GGA | 0.36 | 0.24 | GGA |
| | GGC | 0.23 | | |
| | GGG | 0.17 | | |
| | GGT | 0.24 | | GGT |
| His | CAC | 0.45 | 0.5 | |
| | CAT | 0.55 | | CAT |
| Ile | ATA | 0.27 | 0.3 | |
| | ATC | 0.3 | | ATC |
| | ATT | 0.43 | | ATT |
| Leu | CTA | 0.12 | 0.17 | |
| | CTC | 0.22 | | CTC |
| | CTG | 0.16 | | |
| | CTT | 0.19 | | CTT |
| | TTA | 0.14 | | |
| | TTG | 0.18 | | TTG |
| Lys | AAA | 0.49 | 0.5 | |
| | AAG | 0.51 | | AAG |
| Met | ATG | 1 | 1 | ATG |
| Phe | TTC | 0.56 | 0.5 | TTC |
| | TTT | 0.44 | | |
| Pro | CCA | 0.31 | 0.26 | CCA |
| | CCC | 0.2 | | |
| | CCG | 0.17 | | |
| | CCT | 0.32 | | CCT |
| STOP | TAA | 0.33 | 0.33 | TAA |
| | TAG | 0.42 | TAG | |
| | TGA | 0.24 | | |
| Ser | AGC | 0.12 | 0.17 | |
| | AGT | 0.12 | | |
| | TCA | 0.22 | | TCA |
| | TCC | 0.21 | | TCC |
| | TCG | 0.10 | | |
| | TCT | 0.22 | TCT | |
| Thr | ACA | 0.32 | 0.28 | ACA |
| | ACC | 0.26 | | |
| | ACG | 0.13 | | |
| | ACT | 0.29 | | ACT |
| Trp | TGG | 1 | 1 | TGG |
| Tyr | TAC | 0.46 | 0.5 | |
| | TAT | 0.54 | | TAT |
| Val | GTA | 0.16 | 0.26 | |
| | GTC | 0.25 | | |
| | GTG | 0.26 | | GTG |
| | GTT | 0.33 | GTT | |

TABLE 11

Possible selectable codons based on median values
of maize-specific virus coat/capsid polypeptide
codon usage frequencies

| | | | | |
|---|---|---|---|---|
| | | Maize Viral | Maize Viral | |
| Amino | | Coat (4 Seqs) | Coat | Selectable |
| Acid | Codon | Codon Freq. | Median | Codons |

| | | | | |
|---|---|---|---|---|
| Ala | GCA | 0.38 | 0.24 | GCA |
| | GCC | 0.22 | | |
| | GCG | 0.14 | | |
| | GCT | 0.26 | | GCT |
| Arg | AGA | 0.3 | 0.18 | AGA |
| | AGG | 0.18 | | AGG |
| | CGA | 0.18 | | CGA |
| | CGC | 0.16 | | |
| | CGG | 0.11 | | |
| | CGT | 0.07 | | |
| Asn | AAC | 0.53 | 0.5 | AAC |
| | AAT | 0.47 | | |
| Asp | GAC | 0.45 | 0.5 | |
| | GAT | 0.55 | | GAT |
| Cys | TGC | 0.53 | 0.5 | TGC |
| | TGT | 0.47 | | |
| Gln | CAA | 0.52 | 0.5 | CAA |
| | CAG | 0.48 | | |
| Glu | GAA | 0.44 | 0.5 | |
| | GAG | 0.56 | | GAG |
| Gly | GGA | 0.42 | 0.23 | GGA |
| GGC | 0.18 | | | |
| GGG | 0.23 | | GGG | |
| GGT | 0.18 | | | |
| His | CAC | 0.35 | 0.5 | |
| CAT | 0.65 | | CAT | |
| Ile | ATA | 0.24 | 0.36 | |
| ATC | 0.36 | | ATC | |
| ATT | 0.4 | | ATT | |
| Leu | CTA | 0.12 | 0.15 | |
| CTC | 0.18 | | CTC | |
| CTG | 0.25 | | CTG | |
| CTT | 0.12 | | | |
| TTA | 0.1 | | | |
| TTG | 0.23 | | TTG | |
| Lys | AAA | 0.48 | 0.5 | |
| AAG | 0.52 | | AAG | |
| Met | ATG | 1 | 1 | ATG |
| Phe | TTC | 0.57 | 0.5 | TTC |
| TTT | 0.43 | | | |
| Pro | CCA | 0.32 | 0.28 | CCA |
| | CCC | 0.24 | | |
| | CCG | 0.12 | | |
| | CCT | 0.32 | | CCT |
| STOP | TAA | 0.5 | 0.5 | TAA |
| | TAG | 0 | | |
| | TGA | 0.5 | | TGA |
| Ser | AGC | 0.19 | 0.17 | AGC |
| | AGT | 0.13 | | |
| | TCA | 0.21 | | TCA |
| | TCC | 0.26 | | TCC |
| | TCG | 0.06 | | |
| | TCT | 0.15 | | |
| Thr | ACA | 0.36 | 0.29 | ACA |
| | ACC | 0.27 | | |
| | ACG | 0.06 | | |
| | ACT | 0.31 | | ACT |
| Trp | TGG | 1 | 1 | TGG |
| Tyr | TAC | 0.41 | 0.5 | |
| | TAT | 0.59 | | TAT |
| Val | GTA | 0.15 | 0.25 | |
| | GTC | 0.26 | | GTC |
| | GTG | 0.36 | | GTG |
| | GTT | 0.23 | | |

TABLE 12

Possible selectable codons based on median values
of dicocotyledonous plant virus codon usage
frequencies

| | | | | |
|---|---|---|---|---|
| Amino | | Dicot Viral | Dicot Viral | Selectable |
| Acid | Codon | Codon Freq. | Median | Codons |

| | | | | |
|---|---|---|---|---|
| Ala | GCA | 0.33 | 0.27 | GCA |
| | GCC | 0.21 | | |
| | GCG | 0.13 | | |
| | GCT | 0.33 | | GCT |
| Arg | AGA | 0.34 | 0.13 | AGA |
| | AGG | 0.23 | | AGG |
| | CGA | 0.11 | | |
| | CGC | 0.09 | | |
| | CGG | 0.08 | | |
| | CGT | 0.15 | | CGT |
| Asn | AAC | 0.41 | 0.50 | |
| | AAT | 0.59 | | AAT |
| Asp | GAC | 0.37 | 0.50 | |
| | GAT | 0.63 | | GAT |
| Cys | TGC | 0.41 | 0.50 | |
| | TGT | 0.59 | | TGT |
| Gln | CAA | 0.61 | 0.50 | CAA |
| | CAG | 0.40 | | |
| Glu | GAA | 0.61 | 0.50 | GAA |
| | GAG | 0.39 | | |
| Gly | GGA | 0.35 | 0.24 | GGA |
| | GGC | 0.18 | | |
| | GGG | 0.18 | | |
| | GGT | 0.29 | | GGT |
| His | CAC | 0.43 | | |
| | CAT | 0.57 | | CAT |
| Ile | ATA | 0.31 | 0.31 | ATA |
| | ATC | 0.28 | | |
| | ATT | 0.41 | | ATT |
| Leu | CTA | 0.12 | 0.16 | |
| | CTC | 0.14 | | |
| | CTG | 0.12 | | |
| | CTT | 0.19 | | CTT |
| | TTA | 0.22 | | TTA |
| | TTG | 0.21 | | TTG |
| Lys | AAA | 0.54 | 0.50 | AAA |
| | AAG | 0.46 | | |
| Met | ATG | 1 | 1.00 | ATG |
| Phe | TTC | 0.44 | 0.50 | |
| | TTT | 0.56 | | TTT |
| Pro | CCA | 0.38 | 0.25 | CCA |
| | CCC | 0.18 | | |
| | CCG | 0.12 | | |
| | CCT | 0.31 | | CCT |
| STOP | TAA | 0.46 | 0.30 | TAA |
| | TAG | 0.24 | | |
| | TGA | 0.30 | | TGA |
| Ser | AGC | 0.14 | 0.17 | |
| | AGT | 0.20 | | AGT |
| | TCA | 0.23 | | TCA |
| | TCC | 0.14 | | |
| | TCG | 0.08 | | |
| | TCT | 0.21 | | TCT |
| Thr | ACA | 0.36 | 0.25 | ACA |
| | ACC | 0.20 | | |
| | ACG | 0.14 | | |
| | ACT | 0.31 | | ACT |
| Trp | TGG | 1 | 1.00 | TGG |
| Tyr | TAC | 0.41 | 0.50 | |
| | TAT | 0.59 | | TAT |
| Val | GTA | 0.19 | 0.23 | |
| | GTC | 0.21 | | |
| | GTG | 0.25 | | GTG |
| | GTT | 0.35 | | GTT |

TABLE 13

Possible selectable codons based on median values
of dicocotyledonous plant virus coat/capsid
polypeptide codon usage frequencies

| | | | | |
|---|---|---|---|---|
| | | Dicot Viral | | |
| Amino | | Coat | Dicot Viral | Selectable |
| Acid | Codon | Codon Freq. | Coat Median | Codons |

| | | | | |
|---|---|---|---|---|
| Ala | GCA | 0.24 | 0.255 | |
| | GCC | 0.27 | | GCC\ |
| | GCG | 0.15 | | |
| | GCT | 0.34 | | GCT |
| Arg | AGA | 0.24 | 0.165 | AGA |
| | AGG | 0.22 | | AGG |
| | CGA | 0.12 | | |
| | CGC | 0.1 | | |
| | CGG | 0.11 | | |
| | CGT | 0.21 | | CGT |
| Asn | AAC | 0.44 | 0.5 | |
| | AAT | 0.56 | | AAT |
| Asp | GAC | 0.32 | 0.5 | |
| | GAT | 0.68 | | GAT |
| Cys | TGC | 0.25 | 0.5 | |
| | TGT | 0.75 | | TGT |
| Gln | CAA | 0.59 | 0.5 | CAA |
| | CAG | 0.41 | | |
| Glu | GAA | 0.61 | 0.5 | GAA |
| | GAG | 0.39 | | |
| Gly | GGA | 0.32 | 0.25 | GGA |
| | GGC | 0.2 | | |
| | GGG | 0.18 | | |
| | GGT | 0.3 | | GGT |
| His | CAC | 0.35 | 0.5 | |
| | CAT | 0.65 | | CAT |
| Ile | ATA | 0.39 | 0.35 | ATA |
| | ATC | 0.26 | | |
| | ATT | 0.35 | | ATT |
| Leu | CTA | 0.1 | 0.135 | |
| | CTC | 0.13 | | |
| | CTG | 0.12 | | |
| | CTT | 0.14 | | CTT |
| | TTA | 0.28 | | TTA |
| | TTG | 0.23 | | TTG |
| Lys | AAA | 0.45 | 0.5 | |
| | AAG | 0.55 | | AAG |
| Met | ATG | 1 | 1 | ATG |
| Phe | TTC | 0.47 | 0.5 | |
| | TTT | 0.53 | | TTT |
| Pro | CCA | 0.27 | 0.27 | CCA |
| | CCC | 0.27 | | CCC |
| | CCG | 0.14 | | |
| | CCT | 0.33 | | CCT |
| STOP | TAA | 0.62 | 0.24 | TAA |
| | TAG | 0.14 | | |
| | TGA | 0.24 | | TGA |
| Ser | AGC | 0.15 | 0.165 | |
| | AGT | 0.19 | | AGT |
| | TCA | 0.18 | | TCA |
| | TCC | 0.14 | | |
| | TCG | 0.11 | | |
| | TCT | 0.24 | | TCT |
| Thr | ACA | 0.25 | 0.25 | ACA |
| | ACC | 0.25 | | ACC |
| | ACG | 0.16 | | |
| | ACT | 0.34 | | ACT |
| Trp | TGG | 1 | 1 | TGG |
| Tyr | TAC | 0.37 | 0.5 | |
| | TAT | 0.63 | | TAT |
| Val | GTA | 0.17 | 0.24 | |
| | GTC | 0.23 | | |
| | GTG | 0.25 | | GTG |
| | GTT | 0.35 | | GTT |

### 5.2.3 Frequency Matching Criterion

In another embodiment, altered codons are selected such that the resulting nucleic acid molecule comprising altered codons has a usage frequency for a particular type of amino acid that is the same as or substantially similar to the codon usage frequency in the plant virus, viruses, or subset of nucleic acid molecules therefrom used to create the codon usage frequency table (such as, e.g., those in Tables 2, 3, 4, 6, or 8) for that amino acid. For example, a nucleic acid molecule designed according to the methods of the invention could comprise altered codons such that all of a particular amino acid (e.g., glycine) is encoded by codons in frequencies that is or is substantially similar to plant virus codon usage frequencies (using, e.g., Table 2 glycine would be encoded by GGA, GGT, GGC, GGG at frequencies of 0.37, 0.28, 0.20, and 0.14, respectively).

Codon usage frequencies can be matched in this manner to codon usage frequencies in the plant virus, viruses, or subset of nucleic acid molecules therefrom used to create the codon usage frequency table for one or more types of amino acids. Any number of types of amino acids can be altered to be the same or substantially similar to plant virus codon frequencies. In specific embodiments, at least 2 types of amino acids, at least 5 types of amino acids, at least 8 types of amino acids, at least 12 types of amino acids, at least 18 types of amino acids, or all 20 biologically occurring types of amino acids are encoded by codons that are or are substantially similar to the frequency in one or more plant viruses or a subset of nucleic acid molecules therefrom.

### 5.2.4 Minimum Threshold Criterion

In another embodiment, plant virus codons for which the usage frequency in the plant virus, viruses, or subset of nucleic acid molecules therefrom used to create the codon usage frequency table is 0.09 or less are eliminated as possible altered codons. This procedure eliminates from consideration codons for which a usage frequency in plant viruses is very low (0.09 or less) and thus unlikely to be translated efficiently in plants. Any codon that encodes the same amino acid with a usage frequency of higher than 0.09 can be used as an altered codon to replace the low frequency codon. In specific embodiments, the remaining codons with usage frequencies higher than 0.09 are substituted in a manner that keeps the proportionality between the remaining codons.

Table 14 shows codon usage frequencies for monocotyledonous plant viruses where those codons with frequencies of 0.09 or less (according to Table 2) have been eliminated and the remaining codons have been adjusted proportionally for each amino acid type.

Table 15 shows codon usage frequencies for the maize-specific virus coat/capsid polypeptides where those codons with frequencies of 0.09 or less (according to Table 4) have been eliminated and the remaining codons have been adjusted proportionally for each amino acid type.

Table 16 shows codon usage frequencies for the dicotyledonous plant viruses where those codons with frequencies of 0.09 or less (according to Table 6) have been eliminated and the remaining codons have been adjusted proportionally for each amino acid type.

Table 17 shows codon usage frequencies for the dicotyledonous plant viruses coat/capsid polypeptides where those codons with frequencies of 0.09 or less (according to Table 8) have been eliminated and the remaining codons have been adjusted proportionally for each amino acid type.

For example, in Table 14, there is a single codon, for the amino acid arginine, CGG, for which the original codon usage frequency is not greater than 0.09. The codon usage frequency for CGG is therefore set to 0.00, and the value of 0.09 is redistributed between the frequencies of the remaining codons AGA, AGG, CGA, CGC, and CGT, in proportion to their original codon usage frequencies as indicated. All of the codon usage frequencies for the maize-specific virus nucleic acid molecule coding sequences listed in Table 3 are greater than 0.09, and therefore the codon usage frequencies for maize-specific virus nucleic acid molecule coding sequences remain the same under the 0.09 criterion.

TABLE 14

Monocotyledonous Plant Virus Codon Usage
Frequencies After Eliminating Codons with a Usage
Frequency of ≦0.09 and Adjusting Remaining Codon
Usage Frequencies Proportionally.

| | | | |
|---|---|---|---|
| | | | >0.09 Threshold- |
| | | Monocot Viral | Adjusted Codon |
| Amino Acid | Codon | Codon Freq. | Freq. |

| | | | |
|---|---|---|---|
| Ala | GCA | 0.31 | 0.31 |
| | GCC | 0.21 | 0.21 |
| | GCG | 0.14 | 0.14 |
| | GCT | 0.34 | 0.34 |
| Arg | AGA | 0.32 | 0.35 |
| | AGG | 0.17 | 0.18 |
| | CGA | 0.13 | 0.15 |
| | CGC | 0.13 | 0.15 |
| | CGG | 0.09 | 0.00 |
| | CGT | 0.16 | 0.17 |
| Asn | AAC | 0.42 | 0.42 |
| | AAT | 0.58 | 0.58 |
| Asp | GAC | 0.38 | 0.38 |
| | GAT | 0.62 | 0.62 |
| Cys | TGC | 0.44 | 0.44 |
| | TGT | 0.56 | 0.56 |
| Gln | CAA | 0.58 | 0.58 |
| | CAG | 0.42 | 0.42 |
| Glu | GAA | 0.60 | 0.60 |
| | GAG | 0.40 | 0.40 |
| Gly | GGA | 0.37 | 0.37 |
| | GGC | 0.20 | 0.20 |
| | GGG | 0.14 | 0.14 |
| | GGT | 0.28 | 0.28 |
| His | CAC | 0.43 | 0.43 |
| | CAT | 0.57 | 0.57 |
| Ile | ATA | 0.30 | 0.30 |
| | ATC | 0.29 | 0.29 |
| | ATT | 0.41 | 0.41 |
| Leu | CTA | 0.13 | 0.13 |
| | CTC | 0.14 | 0.14 |
| | CTG | 0.13 | 0.13 |
| | GTT | 0.18 | 0.18 |
| | TTA | 0.21 | 0.21 |
| | TTG | 0.21 | 0.21 |
| Lys | AAA | 0.53 | 0.53 |
| | AAG | 0.47 | 0.47 |
| Met | ATG | 1.00 | 1.00 |
| Phe | TTC | 0.46 | 0.46 |
| | TTT | 0.54 | 0.54 |
| Pro | CCA | 0.38 | 0.38 |
| | CCC | 0.17 | 0.17 |
| | CCG | 0.14 | 0.14 |
| | CCT | 0.31 | 0.31 |
| STOP | TAA | 0.34 | 0.34 |
| | TAG | 0.25 | 0.25 |
| | TGA | 0.41 | 0.41 |
| Ser | AGC | 0.13 | 0.13 |
| | AGT | 0.18 | 0.18 |
| | TCA | 0.24 | 0.24 |
| | TCC | 0.14 | 0.14 |
| | TCG | 0.10 | 0.10 |
| | TCT | 0.21 | 0.21 |
| Thr | ACA | 0.30 | 0.30 |
| | ACC | 0.20 | 0.20 |
| | ACG | 0.16 | 0.16 |
| | ACT | 0.34 | 0.34 |
| Trp | TGG | 1.00 | 1.00 |
| Tyr | TAC | 0.43 | 0.43 |
| | TAT | 0.57 | 0.57 |
| Val | GTA | 0.19 | 0.19 |
| | GTC | 0.21 | 0.21 |
| | GTG | 0.25 | 0.25 |
| | GTT | 0.36 | 0.36 |

TABLE 15

Maize virus coat/capsid polypeptide codon usage
frequencies after eliminating codons with a usage
frequency of ≦0.09 and adjusting remaining codon
usage frequencies proportionally.

| | | | |
|---|---|---|---|
| | | | >0.09 Threshold- |
| | | Maize Viral Coat | Adjusted Codon |
| Amino Acid | Codon | Codon Freq. | Freq. |

| | | | |
|---|---|---|---|
| Ala | GCA | 0.38 | 0.38 |
| | GCC | 0.22 | 0.22 |
| | GCG | 0.14 | 0.14 |
| | GCT | 0.26 | 0.26 |
| Arg | AGA | 0.30 | 0.32 |
| | AGG | 0.18 | 0.19 |
| | CGA | 0.18 | 0.19 |
| | CGC | 0.16 | 0.18 |
| | CGG | 0.11 | 0.12 |
| | CGT | 0.07 | 0.00 |
| Asn | AAC | 0.53 | 0.53 |
| | AAT | 0.47 | 0.47 |
| Asp | GAC | 0.45 | 0.45 |
| | GAT | 0.55 | 0.55 |
| Cys | TGC | 0.53 | 0.53 |
| | TGT | 0.47 | 0.47 |
| Gln | CAA | 0.52 | 0.52 |
| | GAG | 0.48 | 0.48 |
| Glu | GAA | 0.44 | 0.44 |
| | GAG | 0.56 | 0.56 |
| Gly | GGA | 0.42 | 0.42 |
| | GGC | 0.18 | 0.18 |
| | GGG | 0.23 | 0.23 |
| | GGT | 0.18 | 0.18 |
| His | GAG | 0.35 | 0.35 |
| | CAT | 0.65 | 0.65 |
| Ile | ATA | 0.24 | 0.24 |
| | ATC | 0.36 | 0.36 |
| | ATT | 0.40 | 0.40 |
| Leu | CTA | 0.12 | 0.12 |
| | CrC | 0.18 | 0.18 |
| | CTG | 0.25 | 0.25 |
| | CTT | 0.12 | 0.12 |
| | TTA | 0.10 | 0.10 |
| | TTG | 0.23 | 0.23 |
| Lys | AAA | 0.48 | 0.48 |
| | AAG | 0.52 | 0.52 |
| Met | ATG | 1.00 | 1.00 |
| Phe | TTC | 0.57 | 0.57 |
| | TTT | 0.43 | 0.43 |
| Pro | CCA | 0.32 | 0.32 |
| | CCC | 0.24 | 0.24 |
| | CGG | 0.12 | 0.12 |
| | CCT | 0.32 | 0.32 |
| STOP | TAA | 0.50 | 0.50 |
| | TAG | 0.00 | 0.00 |
| | TGA | 0.50 | 0.50 |
| Ser | AGC | 0.19 | 0.20 |
| | AGT | 0.13 | 0.14 |
| | TCA | 0.21 | 0.22 |
| | TCC | 0.26 | 0.28 |
| | TCG | 0.06 | 0.00 |
| | TCT | 0.15 | 0.16 |
| Thr | ACA | 0.36 | 0.39 |
| | ACC | 0.27 | 0.28 |
| | ACG | 0.06 | 0.00 |
| | ACT | 0.31 | 0.33 |
| Trp | TGG | 1.00 | 1.00 |
| Tyr | TAC | 0.41 | 0.41 |
| | TAT | 0.59 | 0.59 |
| Val | GTA | 0.15 | 0.15 |
| | GTC | 0.26 | 0.26 |
| | GTG | 0.36 | 0.36 |
| | GTT | 0.23 | 0.23 |

TABLE 16

Dicotyledonous plant virus codon usage
frequencies, after eliminating codons with a usage
frequency of ≦0.09 and adjusting remaining codon
usage frequencies proportionally.

| | | | |
|---|---|---|---|
| | | | >0.09 Threshold- |
| | | Dicot Viral | Adjusted Codon |
| Amino Acid | Codon | Codon Freq. | Freq. |

| | | | |
|---|---|---|---|
| Ala | GCA | 0.33 | 0.33 |
| | GCC | 0.21 | 0.21 |
| | GCG | 0.13 | 0.13 |
| | GCT | 0.33 | 0.33 |
| Arg | AGA | 0.34 | 0.41 |
| | AGG | 0.23 | 0.28 |
| | CGA | 0.11 | 0.13 |
| | CGC | 0.09 | 0.00 |
| | CGG | 0.08 | 0.00 |
| | CGT | 0.15 | 0.18 |
| Asn | AAC | 0.41 | 0.41 |
| | AAT | 0.59 | 0.59 |
| Asp | GAC | 0.37 | 0.37 |
| | GAT | 0.63 | 0.63 |
| Cys | TGC | 0.41 | 0.41 |
| | TGT | 0.59 | 0.59 |
| Gln | CAA | 0.61 | 0.61 |
| | CAG | 0.40 | 0.40 |
| Glu | GAA | 0.61 | 0.61 |
| | GAG | 0.39 | 0.39 |
| Gly | GGA | 0.35 | 0.35 |
| | GGC | 0.18 | 0.18 |
| | GGG | 0.18 | 0.18 |
| | GGT | 0.29 | 0.29 |
| His | CAC | 0.43 | 0.43 |
| | CAT | 0.57 | 0.57 |
| Ile | ATA | 0.31 | 0.31 |
| | ATC | 0.28 | 0.28 |
| | ATT | 0.41 | 0.41 |
| Leu | CTA | 0.12 | 0.12 |
| | CTC | 0.14 | 0.14 |
| | CTG | 0.12 | 0.12 |
| | CTT | 0.19 | 0.19 |
| | TTA | 0.22 | 0.22 |
| | TTG | 0.21 | 0.21 |
| Lys | AAA | 0.54 | 0.54 |
| | AAG | 0.46 | 0.46 |
| Met | ATG | 1 | 1 |
| Phe | TTC | 0.44 | 0.44 |
| | TTT | 0.56 | 0.56 |
| Pro | CCA | 0.38 | 0.38 |
| | CCC | 0.18 | 0.18 |
| | CCG | 0.12 | 0.12 |
| | CCT | 0.31 | 0.31 |
| STOP | TAA | 0.46 | 0.46 |
| | TAG | 0.24 | 0.24 |
| | TGA | 0.30 | 0.30 |
| Ser | AGC | 0.14 | 0.15 |
| | AGT | 0.20 | 0.22 |
| | TCA | 0.23 | 0.25 |
| | TCC | 0.14 | 0.15 |
| | TCG | 0.08 | 0.00 |
| | TCT | 0.21 | 0.23 |
| Thr | ACA | 0.36 | 0.36 |
| | ACC | 0.20 | 0.20 |
| | ACG | 0.14 | 0.14 |
| | ACT | 0.31 | 0.31 |
| Trp | TGG | 1 | 1 |
| Tyr | TAC | 0.41 | 0.41 |
| | TAT | 0.59 | 0.59 |
| Val | GTA | 0.19 | 0.19 |
| | GTC | 0.21 | 0.21 |
| | GTG | 0.25 | 0.25 |
| | GTT | 0.35 | 0.35 |

TABLE 17

Dicotyledonous plant virus capsid/coat codon usage
frequencies, after eliminating codons with a usage
frequency of ≦0.09 and adjusting remaining codon
usage frequencies proportionally.

| | | | |
|---|---|---|---|
| | | | >0.09 Threshold- |
| | | Dicot Viral Coat | Adjusted Codon |
| Amino Acid | Codon | Codon Freq. | Freq. |

| | | | |
|---|---|---|---|
| Ala | GCA | 0.24 | 0.24 |
| | GCC | 0.27 | 0.27 |
| | GCG | 0.15 | 0.15 |
| | GCT | 0.34 | 0.34 |
| Arg | AGA | 0.24 | 0.24 |
| | AGG | 0.22 | 0.22 |
| | CGA | 0.12 | 0.12 |
| | CGC | 0.10 | 0.10 |
| | CGG | 0.11 | 0.11 |
| | CGT | 0.21 | 0.21 |
| Asn | AAC | 0.44 | 0.44 |
| | AAT | 0.56 | 0.56 |
| Asp | GAC | 0.32 | 0.32 |
| | GAT | 0.68 | 0.68 |
| Cys | TGC | 0.25 | 0.25 |
| | TGT | 0.75 | 0.75 |
| Gln | CAA | 0.59 | 0.59 |
| | GAG | 0.41 | 0.41 |
| Glu | GAA | 0.61 | 0.61 |
| | GAG | 0.39 | 0.39 |
| Gly | GGA | 0.32 | 0.32 |
| | GGC | 0.2 | 0.2 |
| | GGG | 0.18 | 0.18 |
| | GGT | 0.3 | 0.3 |
| His | GAG | 0.35 | 0.35 |
| | CAT | 0.65 | 0.65 |
| Ile | ATA | 0.39 | 0.39 |
| | ATC | 0.26 | 0.26 |
| | ATT | 0.35 | 0.35 |
| Leu | CTA | 0.10 | 0.10 |
| | CTC | 0.13 | 0.13 |
| | CTG | 0.12 | 0.12 |
| | CTT | 0.14 | 0.14 |
| | TTA | 0.28 | 0.28 |
| | TTG | 0.23 | 0.23 |
| Lys | AAA | 0.24 | 0.24 |
| | AAG | 0.27 | 0.27 |
| Met | ATG | 0.15 | 0.15 |
| Phe | TTG | 0.34 | 0.34 |
| | TTT | 0.24 | 0.24 |
| Pro | CCA | 0.22 | 0.22 |
| | CCC | 0.12 | 0.12 |
| | CCG | 0.10 | 0.10 |
| | CCT | 0.11 | 0.11 |
| STOP | TAA | 0.21 | 0.21 |
| | TAG | 0.44 | 0.44 |
| | TGA | 0.56 | 0.56 |
| Ser | AGC | 0.14 | 0.15 |
| | AGT | 0.20 | 0.22 |
| | TCA | 0.23 | 0.25 |
| | TCC | 0.14 | 0.15 |
| | TCG | 0.08 | 0.00 |
| | TCT | 0.21 | 0.23 |
| Thr | ACA | 0.36 | 0.36 |
| | ACC | 0.20 | 0.20 |
| | ACG | 0.14 | 0.14 |
| | ACT | 0.31 | 0.31 |
| Trp | TGG | 1 | 1 |
| Tyr | TAC | 0.41 | 0.41 |
| | TAT | 0.59 | 0.59 |
| Val | GTA | 0.19 | 0.19 |
| | GTC | 0.21 | 0.21 |
| | GTG | 0.25 | 0.25 |
| | GTT | 0.35 | 0.35 |

### 5.2.5 Median Threshold Cut-Off Criterion

In another embodiment, plant virus codons for which the usage frequency in the plant virus, viruses, or subset of nucleic acid molecules therefrom used to create the codon usage frequency table are less than the median codon usage frequency are eliminated as possible altered codons (see Section 5.2.2 for calculation of the median usage frequency). Any codon that encodes the same amino acid with a usage frequency equal to or greater than the median for that particular amino acid can be used as an altered codon to replace the codon. In specific embodiments, the remaining codons with usage frequencies equal to or greater than the median are substituted in a manner that keeps the proportionality between the remaining codons.

Table 18 shows codon usage frequencies for monocotyledonous plant viruses where those codons with frequencies less than the median (according to Table 2) have been eliminated and the remaining codons have been adjusted proportionally for each amino acid type.

Table 19 shows codon usage frequencies for the maize-specific viruses where those codons with frequencies less than the median (according to Table 3) have been eliminated and the remaining codons have been adjusted proportionally for each amino acid type.

Table 20 shows codon usage frequencies for the maize-specific virus coat/capsid polypeptides where those codons with frequencies less than the median (according to Table 4) have been eliminated and the remaining codons have been adjusted proportionally for each amino acid type.

Table 21 shows codon usage frequencies for dicotyledonous plant viruses where those codons with frequencies less than the median (according to Table 6) have been eliminated and the remaining codons have been adjusted proportionally for each amino acid type.

Table 22 shows codon usage frequencies for the dicotyledonous virus coat/capsid polypeptides where those codons with frequencies less than the median (according to Table 8) have been eliminated and the remaining codons have been adjusted proportionally for each amino acid type.

TABLE 18

Monocotyledonous plant virus codon usage
frequencies after eliminating codons with a usage
frequency less than the median and adjusting
remaining codon usage frequencies proportionally.

| | | | | |
|---|---|---|---|---|
| | | Monocot | | Median |
| | | Viral | Monocot Viral | Criterion |
| | | Codon | Median | Codon |
| Amino Acid | Codon | Freq. | Codon Freq. | Freq. |

| | | | | |
|---|---|---|---|---|
| Ala | GCA | 0.31 | 0.26 | 0.48 |
| | GCC | 0.21 | | 0.00 |
| | GCG | 0.14 | | 0.00 |
| | GCT | 0.34 | | 0.52 |
| Arg | AGA | 0.32 | 0.15 | 0.50 |
| | AGG | 0.17 | | 0.27 |
| | CGA | 0.14 | | 0.00 |
| | CGC | 0.14 | | 0.00 |
| | CGG | 0.09 | | 0.00 |
| | CGT | 0.16 | | 0.23 |
| Asn | AAC | 0.42 | 0.50 | 0.00 |
| | AAT | 0.58 | | 1.00 |
| Asp | GAC | 0.38 | 0.50 | 0.00 |
| | GAT | 0.62 | | 1.00 |
| Cys | TGC | 0.44 | 0.50 | 0.00 |
| | TGT | 0.56 | | 1.00 |
| Gln | CAA | 0.58 | 0.50 | 1.00 |
| | CAG | 0.42 | | 0.00 |
| Glu | GAA | 0.60 | 0.50 | 1.00 |
| | GAG | 0.40 | | 0.00 |
| Gly | GGA | 0.37 | 0.24 | 0.57 |
| | GGC | 0.20 | | 0.00 |
| | GGG | 0.14 | | 0.00 |
| | GGT | 0.28 | | 0.43 |
| His | CAC | 0.43 | 0.50 | 0.00 |
| | CAT | 0.57 | | 1.00 |
| Ile | ATA | 0.30 | 0.30 | 0.47 |
| | ATC | 0.29 | | 0.00 |
| | ATT | 0.41 | | 0.53 |
| Leu | CTA | 0.13 | 0.16 | 0.00 |
| | CTC | 0.14 | | 0.00 |
| | CTG | 0.13 | | 0.00 |
| | CTT | 0.18 | | 0.30 |
| | TTA | 0.21 | | 0.35 |
| | TTG | 0.21 | | 0.35 |
| Lys | AAA | 0.53 | 0.50 | 1.00 |
| | AAG | 0.47 | | 0.00 |
| Met | ATG | 1.00 | 1.00 | 1.00 |
| Phe | TTC | 0.46 | 0.50 | 0.00 |
| | TTT | 0.54 | | 1.00 |
| Pro | CCA | 0.38 | 0.24 | 0.55 |
| | CCC | 0.17 | | 0.00 |
| | CCG | 0.14 | | 0.00 |
| | CCT | 0.31 | | 0.45 |
| STOP | TAA | 0.34 | 0.34 | 0.45 |
| | TAG | 0.25 | | 0.00 |
| | TGA | 0.41 | | 0.55 |
| Ser | AGC | 0.13 | 0.16 | 0.00 |
| | AGT | 0.18 | | 0.28 |
| | TCA | 0.24 | | 0.38 |
| | TCC | 0.14 | | 0.00 |
| | TCG | 0.10 | | 0.00 |
| | TCT | 0.21 | | 0.34 |
| Thr | ACA | 0.30 | 0.25 | 0.47 |
| | ACC | 0.20 | | 0.00 |
| | ACG | 0.16 | | 0.00 |
| | ACT | 0.34 | | 0.53 |
| Trp | TGG | 1.00 | 1.00 | 1.00 |
| Tyr | TAC | 0.43 | 0.50 | 0.00 |
| | TAT | 0.57 | | 1.00 |
| Val | GTA | 0.19 | 0.23 | 0.00 |
| | GTC | 0.21 | | 0.00 |
| | GTG | 0.25 | | 0.47 |
| | GTT | 0.36 | | 0.53 |

TABLE 19

Maize virus codon usage frequencies after
eliminating codons with a usage frequency less
than the median and adjusting remaining codon
usage frequencies proportionally.

| | | | | |
|---|---|---|---|---|
| | | | | Median |
| | | Maize Viral | Maize Viral | Criterion |
| | | Codon | Median | Codon |
| Amino Acid | Codon | Freq. | Codon Freq. | Freq. |

| | | | | |
|---|---|---|---|---|
| Ala | GCA | 0.31 | 0.29 | 0.51 |
| | GCC | 0.3 | | 0.49 |
| | GCG | 0.11 | | 0.00 |
| | GCT | 0.28 | | 0.00 |
| Arg | AGA | 0.27 | 0.15 | 0.43 |
| | AGG | 0.17 | | 0.27 |
| | CGA | 0.12 | | 0.00 |
| | CGC | 0.19 | | 0.3 |
| | CGG | 0.12 | | 0.00 |
| | CGT | 0.13 | | 0.00 |
| Asn | AAC | 0.44 | 0.5 | 0.00 |
| | AAT | 0.56 | | 1.00 |
| Asp | GAC | 0.41 | 0.5 | 0.00 |
| | GAT | 0.59 | | 1 |
| Cys | TGC | 0.42 | 0.5 | 0.00 |
| | TGT | 0.58 | | 1.00 |
| Gln | CAA | 0.50 | 0.5 | 0.50 |
| | CAG | 0.50 | | 0.50 |
| Glu | GAA | 0.52 | 0.5 | 1.0 |
| | GAG | 0.48 | | 0.00 |
| Gly | GGA | 0.36 | 0.235 | 0.60 |
| | GGC | 0.23 | | 0.00 |
| | GGG | 0.17 | | 0.00 |
| | GGT | 0.24 | | 0.40 |
| His | CAC | 0.45 | 0.5 | 0.00 |
| | CAT | 0.55 | | 1.0 |
| Ile | ATA | 0.27 | 0.3 | 0.00 |
| | ATC | 0.3 | | 0.41 |
| | ATT | 0.43 | | 0.59 |
| Leu | CTA | 0.12 | 0.17 | 0.00 |
| | CTC | 0.22 | | 0.37 |
| | CTG | 0.16 | | 0.00 |
| | CTT | 0.19 | | 0.33 |
| | TTA | 0.14 | | |
| | TTG | 0.18 | | 0.30 |
| Lys | AAA | 0.49 | 0.5 | 0.00 |
| | AAG | 0.51 | | 1 |
| Met | ATG | 1 | 1 | 1 |
| Phe | TTC | 0.56 | 0.5 | 1 |
| | TTT | 0.44 | | 0.00 |
| Pro | CCA | 0.31 | 0.255 | 0.49 |
| | CCC | 0.2 | | 0.00 |
| | CCG | 0.17 | | 0.00 |
| | CCT | 0.32 | | 0.51 |
| STOP | TAA | 0.33 | 0.33 | 0.43 |
| | TAG | 0.42 | | 0.57 |
| | TGA | 0.24 | | 0.00 |
| Ser | AGC | 0.12 | 0.165 | 0.00 |
| | AGT | 0.12 | | 0.00 |
| | TCA | 0.22 | | 0.34 |
| | TCC | 0.21 | | 0.32 |
| | TCG | 0.10 | | 0.00 |
| | TCT | 0.22 | | 0.34 |
| Thr | ACA | 0.32 | 0.275 | 0.52 |
| | ACC | 0.26 | | 0.00 |
| | ACG | 0.13 | | 0.00 |
| | ACT | 0.29 | | 0.48 |
| Trp | TGG | 1.00 | 1.00 | 1.00 |
| Tyr | TAC | 0.46 | 0.50 | 0.00 |
| | TAT | 0.54 | | 1.00 |
| Val | GTA | 0.16 | 0.255 | 0.00 |
| | GTC | 0.25 | | 0.00 |
| | GTG | 0.26 | | 0.44 |
| | GTT | 0.33 | | 0.56 |

TABLE 20

Maize virus capsid/coat codon usage frequencies
after eliminating codons with a usage frequency
less than the median and adjusting remaining codon
usage frequencies proportionally.

| | | | | |
|---|---|---|---|---|
| | | Maize Viral | Maize Viral | Median |
| | | Coat | Coat | Criterion |
| | | Codon | Median | Codon |
| Amino Acid | Codon | Freq. | Codon Freq. | Freq. |

| | | | | |
|---|---|---|---|---|
| Ala | GCA | 0.38 | 0.24 | 0.60 |
| | GCC | 0.22 | | 0.00 |
| | GCG | 0.14 | | 0.00 |
| | GCT | 0.26 | | 0.40 |
| Arg | AGA | 0.30 | 0.18 | 0.46 |
| | AGG | 0.18 | | 0.27 |
| | CGA | 0.18 | | 0.27 |
| | CGC | 0.16 | | 0.00 |
| | CGG | 0.11 | | 0.00 |
| | CGT | 0.07 | | 0.00 |
| Asn | AAC | 0.53 | 0.50 | 1.00 |
| | AAT | 0.47 | | 0.00 |
| Asp | GAC | 0.45 | 0.50 | 0.00 |
| | GAT | 0.55 | | 1.00 |
| Cys | TGC | 0.53 | 0.50 | 1.00 |
| | TGT | 0.47 | | 0.00 |
| Gln | CAA | 0.52 | 0.50 | 1.00 |
| | CAG | 0.48 | | 0.00 |
| Glu | GAA | 0.44 | 0.50 | 0.00 |
| | GAG | 0.56 | | 1.00 |
| Gly | GGA | 0.42 | 0.23 | 0.65 |
| | GGC | 0.18 | | 0.00 |
| | GGG | 0.23 | | 0.35 |
| | GGT | 0.18 | | 0.00 |
| His | CAC | 0.35 | 0.50 | 0.00 |
| | CAT | 0.65 | | 1.00 |
| Ile | ATA | 0.24 | 0.36 | 0.00 |
| | ATC | 0.36 | | 0.47 |
| | ATT | 0.40 | | 0.53 |
| Leu | CTA | 0.12 | 0.15 | 0.00 |
| | CTC | 0.18 | | 0.27 |
| | CTG | 0.25 | | 0.38 |
| | CTT | 0.12 | | 0.00 |
| | TTA | 0.10 | | 0.00 |
| | TTG | 0.23 | | 0.35 |
| Lys | AAA | 0.48 | 0.50 | 0.00 |
| | AAG | 0.52 | | 1.00 |
| Met | ATG | 1.00 | 1.00 | 1.00 |
| Phe | TTC | 0.57 | 0.50 | 1.00 |
| | TTT | 0.43 | | |
| Pro | CCA | 0.32 | 0.28 | 0.50 |
| | CCC | 0.24 | | 0.00 |
| | CCG | 0.12 | | 0.00 |
| | CCT | 0.32 | | 0.50 |
| STOP | TAA | 0.50 | 0.50 | 0.50 |
| | TAG | 0.00 | | 0.00 |
| | TGA | 0.50 | | 0.50 |
| Ser | AGC | 0.19 | 0.17 | 0.28 |
| | AGT | 0.13 | | 0.00 |
| | TCA | 0.21 | | 0.32 |
| | TCC | 0.26 | | 0.40 |
| | TCG | 0.06 | | 0.00 |
| | TCT | 0.15 | | 0.00 |
| Thr | ACA | 0.36 | 0.29 | 0.54 |
| | ACC | 0.27 | | 0.00 |
| | ACG | 0.06 | | 0.00 |
| | ACT | 0.31 | | 0.46 |
| Trp | TGG | 1.00 | 1.00 | 1.00 |
| Tyr | TAC | 0.41 | 0.50 | 0.00 |
| | TAT | 0.59 | | 1.00 |
| Val | GTA | 0.15 | 0.25 | 0.00 |
| | GTC | 0.26 | | 0.31 |
| | GTG | 0.36 | | 0.42 |
| | GTT | 0.23 | | 0.27 |

TABLE 21

Dicotyledonous plant virus codon usage frequencies
after eliminating codons with a usage frequency
less than the median and adjusting remaining codon
usage frequencies proportionally.

| | | | | |
|---|---|---|---|---|
| | | | | Median |
| | | Dicot Viral | Dicot Viral | Criterion |
| | | Codon | Median | Codon |
| Amino Acid | Codon | Freq. | Codon Freq. | Freq. |

| | | | | |
|---|---|---|---|---|
| Ala | GCA | 0.33 | 0.27 | 0.50 |
| | GCC | 0.21 | | 0.00 |
| | GCG | 0.13 | | 0.00 |
| | GCT | 0.33 | | 0.50 |
| Arg | AGA | 0.34 | 0.13 | 0.47 |
| | AGG | 0.23 | | 0.32 |
| | CGA | 0.11 | | 0.00 |
| | CGC | 0.09 | | 0.00 |
| | CGG | 0.08 | | 0.00 |
| | CGT | 0.15 | | 0.21 |
| Asn | AAC | 0.41 | 0.5 | 0.00 |
| | AAT | 0.59 | | 1 |
| Asp | GAC | 0.37 | 0.5 | 0.00 |
| | GAT | 0.63 | | 1 |
| Cys | TGC | 0.41 | 0.5 | 0.00 |
| | TGT | 0.59 | | 0.59 |
| Gln | CAA | 0.61 | 0.5 | 1 |
| | CAG | 0.40 | | 0.00 |
| Gln | GAA | 0.61 | 0.5 | 1 |
| | CAG | 0.39 | | 0.00 |
| Gly | GGA | 0.35 | 0.24 | 0.55 |
| | GGC | 0.18 | | 0.00 |
| | GGG | 0.18 | | 0.00 |
| | GGT | 0.29 | | 0.45 |
| His | CAC | 0.43 | | 0.00 |
| | CAT | 0.57 | | 1 |
| Ile | ATA | 0.31 | 0.31 | 0.43 |
| | ATC | 0.28 | | 0.00 |
| | ATT | 0.41 | | 0.57 |
| Leu | CTA | 0.12 | 0.16 | 0.00 |
| | CTC | 0.14 | | 0.00 |
| | CTG | 0.12 | | 0.00 |
| | CTT | 0.19 | | 0.3 |
| | TTA | 0.22 | | 0.36 |
| | TTG | 0.21 | | 0.34 |
| Lys | AAA | 0.54 | 0.50 | 1 |
| | AAG | 0.46 | | 0.00 |
| Met | ATG | 1.00 | 1.00 | 1.00 |
| Phe | TTC | 0.44 | 0.50 | 0.00 |
| | TTT | 0.56 | | 1 |
| Pro | CCA | 0.38 | 0.25 | 0.54 |
| | CCC | 0.18 | | 0.00 |
| | CCG | 0.12 | | 0.00 |
| | CCT | 0.31 | | 0.46 |
| STOP | TAA | 0.46 | 0.30 | 0.60 |
| | TAG | 0.24 | | 0.00 |
| | TGA | 0.30 | | 0.40 |
| Ser | AGC | 0.14 | 0.17 | 0.00 |
| | AGT | 0.20 | | 0.32 |
| | TCA | 0.23 | | 0.33 |
| | TCC | 0.14 | | 0.00 |
| | TCG | 0.08 | | 0.00 |
| | TCT | 0.21 | | 0.35 |
| Thr | ACA | 0.36 | 0.25 | 0.54 |
| | ACC | 0.20 | | |
| | ACG | 0.14 | | |
| | ACT | 0.31 | | 0.46 |
| Trp | TGG | 1 | 1 | 1 |
| Tyr | TAC | 0.41 | 0.5 | 0.00 |
| | TAT | 0.59 | | 1 |
| Val | GTA | 0.19 | 0.23 | 0.00 |
| | GTC | 0.21 | | 0.00 |
| | GTG | 0.25 | | 0.42 |
| | GTT | 0.35 | | 0.58 |

TABLE 22

Dicotyledonous plant virus capsid/coat codon usage
frequencies after eliminating codons with a usage
frequency less than the median and adjusting
remaining codon usage frequencies proportionally.

| | | | | |
|---|---|---|---|---|
| | | Dicot Viral | Dicot Viral | Median |
| | | Coat | Coat | Criterion |
| | | Codon | Median | Codon |
| Amino Acid | Codon | Freq. | Codon Freq. | Freq. |

| | | | | |
|---|---|---|---|---|
| Ala | GCA | 0.24 | 0.255 | 0.00 |
| | GCC | 0.27 | | 0.44 |
| | GCG | 0.15 | | 0.00 |
| | GCT | 0.34 | | 0.56 |
| Arg | AGA | 0.24 | 0.165 | 0.36 |
| | AGG | 0.22 | | 0.33 |
| | CGA | 0.12 | | 0.00 |
| | CGC | 0.10 | | 0.00 |
| | CGG | 0.11 | | 0.00 |
| | CGT | 0.21 | | 0.31 |
| Asn | AAC | 0.44 | 0.50 | 0.00 |
| | AAT | 0.56 | | 1.00 |
| Asp | GAC | 0.32 | 0.50 | 0.00 |
| | GAT | 0.68 | | 1.00 |
| Cys | TGC | 0.25 | 0.50 | 0.00 |
| | TGT | 0.75 | | 1.00 |
| Gln | CAA | 0.59 | 0.50 | 1 |
| | CAG | 0.41 | | 0.00 |
| Glu | GAA | 0.61 | 0.50 | 1 |
| | GAG | 0.39 | | 0.00 |
| Gly | GGA | 0.32 | 0.25 | 0.52 |
| | GGC | 0.2 | | 0.00 |
| | GGG | 0.18 | | 0.00 |
| | GGT | 0.3 | | 0.48 |
| His | CAC | 0.35 | 0.50 | 0.00 |
| | CAT | 0.65 | | 1.00 |
| Ile | ATA | 0.39 | 0.35 | 0.53 |
| | ATC | 0.26 | | 0.00 |
| | ATT | 0.35 | | 0.47 |
| Leu | CTA | 0.10 | 0.135 | 0.00 |
| | CTC | 0.13 | | 0.00 |
| | CTG | 0.12 | | 0.00 |
| | CTT | 0.14 | | 0.22 |
| | TTA | 0.28 | | 0.43 |
| | TTG | 0.23 | | 0.35 |
| Lys | AAA | 0.45 | 0.50 | 0.00 |
| | AAG | 0.55 | | 1.00 |
| Met | ATG | 1.00 | 1 | 1.00 |
| Phe | TTC | 0.47 | 0.50 | 0.00 |
| | TTT | 0.53 | | 1.00 |
| Pro | CCA | 0.27 | 0.27 | 0.31 |
| | CCC | 0.27 | | 0.31 |
| | CCG | 0.14 | | 0.00 |
| | CCT | 0.33 | | 0.38 |
| STOP | TAA | 0.62 | 0.24 | 0.72 |
| | TAG | 0.14 | | 0.00 |
| | TGA | 0.24 | | 0.28 |
| Ser | AGC | 0.15 | 0.165 | 0.00 |
| | AGT | 0.19 | | 0.32 |
| | TCA | 0.18 | | 0.29 |
| | TCC | 0.14 | | 0.00 |
| | TCG | 0.11 | | 0.00 |
| | TCT | 0.24 | | 0.39 |
| Thr | ACA | 0.25 | 0.25 | 0.29 |
| | ACC | 0.25 | | 0.29 |
| | ACG | 0.16 | | 0.00 |
| | ACT | 0.34 | | 0.42 |
| Trp | TGG | 1.00 | 1 | 1.00 |
| Tyr | TAC | 0.37 | 0.5 | 0.00 |
| | TAT | 0.63 | | 1.00 |
| Val | GTA | 0.17 | 0.24 | 0.00 |
| | GTC | 0.23 | | 0.00 |
| | GTG | 0.25 | | 0.42 |
| | GTT | 0.35 | | 0.58 |

### 5.3 Non-Plant Virus Codon Biased Based Modifications

In designing plant virus codon-biased nucleic acid molecule coding sequences according to the present invention, after codon selection based on the criteria illustrated above, additional nucleotide sequence modifications can be made to i) decrease an unfavorable characteristic of the nucleic acid molecule and/or ii) further increase expression of a polypeptide encoded by a plant virus codon-biased nucleic acid molecule coding sequence. Thus, although nucleic acid molecules designed using the methods of the invention may not comprise all of the optimized codons due to considerations listed below, they will be enriched in codons that are more frequently used in plant viruses than an unaltered nucleic acid molecule.

Preferably, the non-codon biased based modification does not alter any amino acid that is encoded by the nucleic acid molecule. In embodiments where an amino acid is changed due to non-codon biased based modifications in the nucleic acid molecule, such a change should preferably keep at least some of the properties of the original amino acid (e.g., charge, size, etc.)

In one embodiment, the Kozak context is changed. The Kozak context is the nucleotide sequence near the start codon ATG. In maize and many cereals the preferred Kozak context is ATGG. This fourth base of the nucleic acid molecule coding sequence is dictated by the encoded second amino acid. If already present, no changes are needed. To create an ATGG Kozak context (Kozak optimization) if it does not exist, however, may require a change in the second amino acid. In polypeptides that are processed at the N-terminus, such as having their N-terminus transit peptide removed, this would not affect the function of the mature polypeptide. Changing the second amino acid to one that has an initial G codon and which is the most chemically similar amongst such amino acids with initial G codons is the preferred approach, however in embodiments in which the second amino acid is altered it is important to make sure that the polypeptide retains critical properties (e.g. enzyme activity, antigenicity, etc.).

In another embodiment, intronic-like sequences created by addition of the altered codons are abolished. In selecting codons for a plant virus codon-biased nucleic acid molecule coding sequence, one may inadvertently introduce one or more potentially functional intronic sequences. Upon expression of the encoded transcript in cells, these introns may be spliced out, causing an internal deletion of a portion of the coding region or reading frame shift. Consequently, it is desirable to eliminate any sites that are highly likely to be intronic. Intron splice-donor sites generally follow the GT-AG rule. In a given nucleic acid molecule coding sequence there are likely to be many GT and AG sites, and thus many potential introns. However, not all of these GT-AG combinations are likely to reveal a functional intron.

Gene prediction software has been developed that uses sophisticated heuristics to decide which if any potential GT-AG combinations represent likely intron splice-donor sites. See, for example, Brendel et al. (2004) Bioinformatics. 20(7): 1157-69; Hermann et al. (1996) Nucl. Acids Res. 24(23): 4709-4718; Brendel et al. (1998) Nucl. Acids Res. 26(20): 4748-4757; Usuka et al. (2000) Bioinformatics 16(3), 203-211; Usuka et al. (2000) J. Mol. Biol. 297(5): 1075-1085, herein incorporated by reference. Programs such as GeneSeqr are particularly useful. GeneSeqr was developed by Volker Brendel at ISU. The output of the GeneSeqr program indicates whether there are any highly likely intron sites in the nucleic acid molecule coding sequence. Information about the GeneSeqr program and the interpretation of its output can be found in the art (e.g., Schlueter et al., 2003, Nucl. Acids Res. 31:3597-3600). Another program that can be used for this purpose is FgenesH. By using more than one program elimination of all cryptic splice sites is more likely. Removing these potential introns can be done by changing either the GT or AG sequences bordering the introns. This can be done in such a manner, if possible, so as to not affect amino acid usage. Another approach to effect removal of these cryptic splice sites is to change bordering nucleotides on the putative intronic side of the putative cryptic splice site borders.

In another embodiment, sequences which encode a putative poly-adenylation signal is changed to prevent spurious polyadenylation within the nucleic acid molecule coding sequence. Such sites include the following sequences: AATAAA, ATAAAA, and AATAAT.

In another embodiment, secondary RNA structures are decreased or eliminated. Transcripts that form hairpin RNA structures may be more likely to be targeted for degradation and/or translational arrest. Consequently, it is desirable to subject the nucleic acid molecule coding sequence to a secondary RNA structure prediction program and then to disrupt any RNA structures predicted to be unusually stable by altering the sequence. Any RNA secondary structure prediction program known in the art may be used. One commonly used program is the GCG Wisconsin package program STEMLOOP. This program is desirable because it ranks the stem-loop structures from the highest to lowest probability to form a secondary structure (essentially from length and quality), and gives their coordinates in the sequence. Among the output results one looks for any standout predicted RNA structures that are unusually long and of high quality. These are to be disrupted by base changes, often in the third position (“wobble” position) of codons, so as not to change amino acid sequence.

In another embodiment, sequences that decrease RNA stability are changed. Certain sequence motifs are known to destabilize mRNA and are therefore sought out and eliminated where possible. In a specific embodiment, “AUUUA” sequences can lead to an increased rate of mRNA degradation. As such, the plant virus codon-biased nucleic acid molecule coding sequences of the invention can be searched for any sequences that are “ATTTA”, and these can be altered without changing the amino acid sequence, if possible.

In another specific embodiment, the presence of “Downstream Element” (DST) mRNA destabilizing sites may dispose mRNA transcripts towards degradation and high turn over. The DST elements follow the general pattern of ATAGAT-N(15)-GTA. Sequences following the pattern ATAGAT-N(10-20)-GTA can be eliminated.

In another specific embodiment, long poly-A or poly-T sequences may contribute to mRNA instability. Consequently, long stretches of one nucleotide, especially long stretches of As or Ts, should be altered. Stretches of three or more of the same nucleotide are sought for mitigation, however, more preferably, stretches of four or more are changed. Additionally, stretches of AT-rich sequences may also be changed.

In another embodiment, the nucleic acid molecule is modified such that the polypeptide of interest is the only polypeptide expressed from the nucleic acid molecule. It is desired that a transgene only express the desired gene product from the desired open reading frame (ORF), which will be the frame 1 translation. Spurious polypeptide products arising from any of the other 5 frame translations are not desired therefore the nucleic acid molecule of the invention can be altered such that the possibility of spurious ORF translation is mitigated. The nucleic acid molecule designed using the methods of the invention is subjected to a 6-frame ORF prediction analysis. The lengths of the ORFs in the five frames not intending to encode a polypeptide can be measured. Those ORFs, particularly those with a potential methionine start codon (i.e. close to a Kozak consensus sequence) and those in frames 2 and 3 that are particularly long (such as longer than 50-100 codons or whichever cut-off threshold is desired) should be shortened by introduction of stop codons or removal of potential start codons.

In another embodiment, restriction enzyme recognition sites can be added to the nucleic acid molecule.

### 5.4 Design of Codon-Biased Nucleic Acid Molecules

The present invention encompasses nucleic acid molecules designed according to the methods of the invention. Nucleic acid molecules encoding polypeptides of interest for expression in plants can be designed for improved expression in plants according to the methods of the present invention. Once codon usage frequency tables are generated for the particular virus, group of viruses, or subset of nucleic acid molecules therefrom of interest, the codons originally present in the nucleic acid molecule can be assessed for their frequency values as compared to plant viruses. Criteria according to Section 5.2 are used to choose which codons can be changed and which codons can be substituted (e.g., altered codons) for them. Nucleic acid molecules comprising altered codons include 5%, 10%, 20%, 30%, 50%, 75%, 85%, 95% altered codons relative to the unaltered (original) nucleic acid molecule. However, codon usage frequencies are not the sole criteria for nucleic acid molecule modification (see Section 5.3).

Any codon in the nucleic acid molecule can be substituted for an altered codon that has a higher usage frequency in plant viruses. In some embodiments the altered codons are “front loaded”, i.e., the number of altered codons is greater in a first portion of the nucleic acid molecule than in a second portion of the nucleic acid molecule, wherein the first portion is 5′ to the second portion. In a more specific embodiment, the first portion and second portion of the nucleic acid molecule are equal, thus there are more altered codons in the 5′ half of the nucleic acid molecule. In another specific embodiment, the first portion is one third of the nucleic acid molecule and comprises an equal number or more altered codons than the second portion which is two thirds of the nucleic acid molecule. Thus, the 5′ third of the nucleic acid molecule has the same number or more altered codons than the 3′ two thirds. In another specific embodiment, the first portion is one quarter of the nucleic acid molecule and comprises an equal number or more altered codons than the second portion which is three quarters of the nucleic acid molecule. Thus, the 5′ quarter of the nucleic acid molecule has the same number or more altered codons than the 3′ three quarters.

Preferably, nucleic acid molecules comprising altered codons encode a polypeptide with a sequence that is identical to that of a polypeptide encoded by an unaltered nucleic acid molecule. In embodiments where the nucleic acid molecule comprising altered codons encodes a polypeptide that is not identical in sequence to an unaltered polypeptide, the altered amino acids are preferably conservative substitutions. Standard techniques known to those skilled in the art can be used to assay any differences in polypeptide function between a polypeptide with amino acid substitutions due to codon alteration and a polypeptide encoded by an unaltered nucleic acid molecule. Preferably, there are no changes in polypeptide function. However, slight alterations in function are tolerable if such polypeptides have substantially similar functions (e.g., are within one standard deviation of each other).

In a specific embodiment, the nucleic acid molecules of the invention encode insecticidal polypeptides. In a more specific embodiment, the insecticidal polypeptides are from Bacillus thuringiensis or Rhyzopus oryzae. In an even more specific embodiment, the insecticidal polypeptides from Bacillus thuringiensis are the 437N and Cry polypeptides. In another more specific embodiment, the insecticidal polypeptide from Rhyzopus oryzae is a insecticidal lipase polypeptide. The present invention encompasses nucleic acid molecules designed according to the methods including, but not limited to, SEQ ID NOS:1 and 3 that encode codon optimized 437N and insecticidal lipase, respectively. Polypeptides encoded by the nucleic acid molecules of the invention are also encompassed by the invention including, but not limited to, SEQ ID NOS:2 and 4 that are codon optimized 437N and insecticidal lipase, respectively.

Also encompassed by the present invention are vectors, host cells, transgenic plants and progeny thereof comprising nucleic acid molecules made according to the methods of the invention.

The present invention does not encompass nucleic acid molecules that encode naturally occurring nucleic acid molecules (e.g., those found in nature and expressed from the genomes of non-transgenic organisms). The present invention also does not encompass nucleic acid molecules of SEQ ID NOS:7-16.

### 5.4.1 Construction of Codon-Biased Nucleic Acid Molecules

The nucleic acid molecules to be altered according to the methods of the invention may be obtained, and their nucleotide sequence determined, by any method known in the art. Such a nucleic acid molecule may be assembled from chemically synthesized oligonucleotides (e.g., as described in Kutmeier et al., 1994, BioTechniques 17:242), which, briefly, involves the synthesis of overlapping oligonucleotides containing portions of the sequence encoding the polypeptide, annealing and ligating of those oligonucleotides, and then amplification of the ligated oligonucleotides by PCR. Alternatively, a nucleic acid molecule may be generated from nucleic acid molecule from a suitable source. If a clone containing a nucleic acid molecule encoding a particular polypeptide is not available, but the sequence of the polypeptide is known, a nucleic acid molecule encoding the polypeptide may be chemically synthesized or obtained from a suitable source (e.g., a cDNA library generated from, or nucleic acid molecule, preferably poly A+ RNA, isolated from, any tissue or cells expressing the polypeptide of interest) by PCR amplification using synthetic primers hybridizable to the 3′ and 5′ ends of the sequence or by cloning using an oligonucleotide probe specific for the particular sequence to identify, e.g., a cDNA clone from a cDNA library that encodes the polypeptide of interest. Amplified nucleic acid molecules generated by PCR may then be cloned into replicable cloning vectors using any method well known in the art.

Once the nucleic acid molecule is obtained it may be manipulated using methods well known in the art for the manipulation of nucleotide sequences, e.g., recombinant DNA techniques, site directed mutagenesis, PCR, etc. (see, for example, the techniques described in Sambrook et al., 1990, Molecular Cloning, A Laboratory Manual, 2d Ed., Cold Spring Harbor Laboratory, Cold Spring Harbor, N.Y.; Ausubel et al., eds., 1998, Current Protocols in Molecular Biology, John Wiley & Sons, NY; U.S. Pat. Nos. 5,789,166 and 6,391,548) to generate the nucleic acid molecules comprising altered codons. Standard techniques known to those skilled in the art can be used to introduce mutations in the nucleotide sequence, or fragment thereof, including, e.g., site-directed mutagenesis and PCR-mediated mutagenesis, such that codons are altered to those codons having a higher usage frequency in plant viruses. Preferably, the nucleic acid molecules comprising altered codons include 5%, 10%, 20%, 30%, 50%, 75%, 85%, 95% altered codons relative to the unaltered (original) nucleic acid molecule. Preferably, nucleic acid molecules comprising altered codons encode a polypeptide with a sequence that is identical to that of a polypeptide encoded by an unaltered nucleic acid molecule. In embodiments where the nucleic acid molecule comprising altered codons encodes a polypeptide that is not identical in sequence to an unaltered polypeptide, the altered amino acids are preferably conservative substitutions. Standard techniques known to those skilled in the art can be used to assay any differences in polypeptide function between a polypeptide with amino acid substitutions due to codon alteration and a polypeptide encoded by an unaltered nucleic acid molecule. Preferably, there are no changes in polypeptide function. However, slight alterations in function are tolerable if such polypeptides have substantially similar functions (e.g., are within one standard deviation of each other).

Once a nucleic acid molecule has been designed and obtained, a vector comprising the nucleic acid molecule may be produced by recombinant DNA technology using techniques well known in the art. Methods which are well known to those skilled in the art can be used to construct vectors, including expression vectors, containing nucleic acid molecules comprising altered codons operably linked to appropriate transcriptional and translational control signals.

In some embodiments, nucleic acid molecules of the invention are in expression vectors. In other embodiments, nucleic acid molecules of the invention are in vectors meant to facilitate integration into plant DNA. Vectors comprising nucleic acid molecules of the invention may also comprise regions that initiate or terminate transcription and/or translation. The elements of these regions may be naturally occurring (either heterologous or native to the plant host cell) or synthetic.

A number of promoters can be used in the practice of the invention. For example, a nucleic acid molecule of the invention can be combined with constitutive, tissue-preferred, inducible, or other promoters for expression in the host organism. In one embodiment, the promoter is a constitutive promoter including, but not limited to, the core promoter of the Rsyn7 promoter and other constitutive promoters disclosed in WO 99/43838 and U.S. Pat. No. 6,072,050; the core CaMV 35S promoter (Odell et al. (1985) Nature 313:810-812); rice actin (McElroy et al. (1990) Plant Cell 2:163-171); ubiquitin (Christensen et al. (1989) Plant Mol. Biol. 12:619-632 and Christensen et al. (1992) Plant Mol. Biol. 18:675-689); pEMU (Last et al. (1991) Theor. Appl. Genet. 81:581-588); MAS (Velten et al. (1984) EMBO J. 3:2723-2730); ALS promoter (U.S. Pat. No. 5,659,026), and the like. Other constitutive promoters include, for example, those discussed in U.S. Pat. Nos. 5,608,149; 5,608,144; 5,604,121; 5,569,597; 5,466,785; 5,399,680; 5,268,463; 5,608,142; and 6,177,611.

In another embodiment, the promoter is an inducible promoter including, but not limited to, wound-inducible promoters (such as those promoters associated with, e.g., potato polypeptidease inhibitor gene, wun1, wun2, win1, win2, systemin, WIP1, MPI gene); pathogen-inducible promoters (such as those promoters associated with, e.g., pathogenesis-related polypeptides, SAR polypeptides, beta-1,3-glucanase, chitinase, PRms gene (see Redolfi et al. (1983) Neth. J. Plant Pathol. 89:245-254; Uknes et al. (1992) Plant Cell 4:645-656; and Van Loon (1985) Plant Mol. Virol. 4:111-116, WO 99/43819, Cordero et al. (1992) Physiol. Mol. Plant Path. 41:189-200, U.S. Pat. No. 5,750,386)); chemical-regulated promoters (such as those promoters associated with, e.g., maize 1n2-2 promoter, maize GST promoter, tobacco PR-1a promoter (see also Schena et al. (1991) Proc. Natl. Acad. Sci. USA 88:10421-10425; McNellis et al. (1998) Plant J. 14(2):247-257); Gatz et al. (1991) Mol. Gen. Genet. 227:229-237, and U.S. Pat. Nos. 5,814,618 and 5,789,156)).

In another embodiment, the promoter is tissue-preferred promoter including, but not limited to, those described in Kawamata et al. (1997) Plant Cell Physiol. 38(7):792-803; Hansen et al. (1997) Mol. Gen Genet. 254(3):337-343; Russell et al. (1997) Transgenic Res. 6(2):157-168; Rinehart et al. (1996) Plant Physiol. 112(3):1331-1341; Van Camp et al. (1996) Plant Physiol. 112(2):525-535; Canevascini et al. (1996) Plant Physiol. 112(2):513-524; Yamamoto et al. (1994) Plant Cell Physiol. 35(5):773-778; Lam (1994) Results Probl. Cell Differ. 20:181-196; Orozco et al. (1993) Plant Mol. Biol. 23(6):1129-1138; Matsuoka et al. (1993) Proc Natl. Acad. Sci. USA 90(20):9586-9590; and Guevara-Garcia et al. (1993) Plant J. 4(3):495-505.

In another embodiment, the promoter is tissue-specific promoter including, but not limited to, promoters specific for leaf (Yamamoto et al. (1997) Plant J. 12(2):255-265; Kwon et al. (1994) Plant Physiol. 105:357-67; Yamamoto et al. (1994) Plant Cell Physiol. 35(5):773-778; Gotor et al. (1993) Plant J. 3:509-18; Orozco et al. (1993) Plant Mol. Biol. 23(6):1129-1138; and Matsuoka et al. (1993) Proc. Natl. Acad. Sci. USA 90(20):9586-9590); root (Hire et al. (1992) Plant Mol. Biol. 20(2):207-218, Keller and Baumgartner (1991) Plant Cell 3(10):1051-1061, Sanger et al. (1990) Plant Mol. Biol. 14(3):433-443, Miao et al. (1991) Plant Cell 3(1):11-22, Bogusz et al. (1990) Plant Cell 2(7):633-641, Kuster et al. (1995) Plant Mol. Biol. 29(4):759-772, Capana et al. (1994) Plant Mol. Biol. 25(4):681-691, U.S. Pat. Nos. 5,837,876; 5,750,386; 5,633,363; 5,459,252; 5,401,836; 5,110,732; and 5,023,179); seed (including those promoters of, e.g., Cim1, cZ19B1, myo-inositol-1-phosphate synthase, Gama-zein, Glob-1, celA, bean β-phaseolin, napin, β-conglycinin, soybean lectin, cruciferin, maize 15 kDa zein, 22 kDa zein, 27 kDa zein, g-zein, waxy, shrunken 1, shrunken 2, and globulin 1 (see also Thompson et al. (1989) BioEssays 10:108, WO 00/12733, WO 00/11177)).

In another embodiment, the promoter is a low level expression promoter (e.g., causes expression of about 1/1000 transcripts to about 1/100,000 transcripts to about 1/500,000 transcripts) including, but not limited to, WO 99/43838, U.S. Pat. No. 6,072,050, U.S. Pat. Nos. 5,608,149; 5,608,144; 5,604,121; 5,569,597; 5,466,785; 5,399,680; 5,268,463; 5,608,142; and 6,177,611.

### 5.5 Polypeptides of the Invention

Any polypeptide known in the art can be expressed in a plant using the methods of the present invention to design the nucleic acid molecule encoding the polypeptide. The polypeptide may occur in nature, be a man-made modification of a naturally occurring polypeptide, be a polypeptide that is designed entirely de novo, or any combination thereof. In preferred embodiments, expression of the polypeptide encoded by a nucleic acid molecule of the present invention alters at least one phenotype of the plant expressing the polypeptide. In specific embodiments, the phenotype of the plant expressing the polypeptide is altered as compared to a control plant. The control plant either i) does not contain and/or express the nucleic acid molecule encoding the polypeptide of interest or ii) contains and/or expresses the nucleic acid molecule encoding the polypeptide of interest but does not comprise any altered codons.

Examples of phenotypes that can be altered by expression of a polypeptide encoded by a nucleic acid molecule of the invention including, but not limited to: insect resistance/tolerance (e.g., by expressing Bacillus 437N or Cry polypeptides or Rhyzopus insecticidal lipase polypeptides), disease resistance/tolerance (e.g., by expressing Pps-AMP1), nematode resistance/tolerance (e.g., by expressing cyclostine), drought resistance/tolerance (e.g., by expressing IPT), salt tolerance, heavy metal tolerance and detoxification, herbicide resistance/tolerance (e.g., by expressing glyphosate acetyl transferase or acetolactate synthase), low phytate content, high-efficiency nitrogen usage, yield enhancement, increased yield stability, improved nutritional content, increased sugar content, improved growth and vigor, improved digestibility, expression of therapeutic polypeptides, synthesis of non-polypeptide pharmaceuticals, expression of selectable marker polypeptides (e.g., GAT), expression of reporter polypeptides (e.g., GUS), and male sterility.

In a specific embodiment, insecticidal polypeptides encoded by plant virus codon-biased nucleic acid molecules are from Bacillus thuringiensis or Rhyzopus oryzae. In a more specific embodiment, the Bacillus thuringiensis insecticidal polypeptide is the 437N or CRY polypeptide. In another more specific embodiment, the Rhyzopus oryzae polypeptide is the insecticidal lipase polypeptide.

### 5.6 Plants

Nucleic acid molecules designed using methods of the present invention can be used for transformation of any plant species, including, but not limited to, monocots and dicots. Examples of plants of interest include, but are not limited to, corn (Zea mays), Brassica sp. (e.g., B. napus, B. rapa, B. juncea), particularly those Brassica species useful as sources of seed oil, alfalfa (Medicago sativa), rice (Oryza saliva), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), palm (Elaeis guinnesis), flax (Linum uistatissimum), castor (Ricinus communis), guar (Athamantha sicula), lentil (Lens culinaris), fenugreek (Trigonella corniculata), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats, barley, vegetables, ornamentals, and conifers.

Examples of vegetables include, but are not limited to, tomatoes (Lycopersicon esculentum), lettuce (Lactuca sativa), green beans (Phaseolus vulgaris), lima beans (Phaseolus limensis), peas (Lathyrus spp.), locust bean (Ceratonia siliqua), cowpea (Vigna unguiculata), mungbean (Vigna radiata), fava bean (Vicia faba), chickpea (Cicer arietinum), and members of the genus Cucumis such as cucumber (C. sativus), cantaloupe (C. cantalupensis), and musk melon (C. melo).

Examples of ornamentals include, but are not limited to, azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherrima), and chrysanthemum.

Examples of conifers include, but are not limited to, pines such as loblolly pine (Pinus taeda), slash pine (Pinus elliotii), ponderosa pine (Pinus ponderosa), lodgepole pine (Pinus contorta), and Monterey pine (Pinus radiata); Douglas fir (Pseudotsuga menziesii); Western hemlock (Tsuga canadensis); Sitka spruce (Picea glauca); redwood (Sequoia sempervirens); true firs such as silver fir (Abies amabilis) and balsam fir (Abies balsamea); and cedars such as Western red cedar (Thuja plicata) and Alaska yellow cedar (Chamaecyparis nootkatensis).

Preferably, plants of the present invention are crop plants (e.g., corn, alfalfa, sunflower, Brassica, soybean, cotton, safflower, peanut, sorghum, wheat, millet, tobacco, rice, etc.).

Also encompassed by the present invention are transgenic plants and progeny thereof comprising nucleic acid molecule molecules made according to the methods of the invention. The invention further relates to plant propagating material of a transformed plants including, but not limited to, seeds, tubers, corms, bulbs, leaves, and cuttings of roots and shoots.

### 5.6.1 Transformation of Plants

Any method known in the art can be used for transforming a plant or plant cell with a nucleic acid molecule designed according to the methods of the present invention. Nucleic acid molecules can be incorporated into plant DNA (e.g., genomic DNA or chloroplast DNA) or be maintained without insertion into the plant DNA (e.g., through the use of artificial chromosomes). Suitable methods of introducing nucleotide sequences into plant cells include microinjection (Crossway et al. (1986) Biotechniques 4:320-334); electroporation (Riggs et al. (1986) Proc. Natl. Acad. Sci. USA 83:5602-5606; D'Halluin et al. (1992) Plant Cell 4:1495-1505); Agrobacterium-mediated transformation (U.S. Pat. Nos. 5,563,055 and 5,981,840, Osjoda et al. (1996) Nature Biotechnology 14:745-750); direct gene transfer (Paszkowski et al. (1984) EMBO J. 3:2717-2722); ballistic particle acceleration (Sanford et al., U.S. Pat. No. 4,945,050; Tomes et al., U.S. Pat. No. 5,879,918; Tomes et al., U.S. Pat. No. 5,886,244; Bidney et al., U.S. Pat. No. 5,932,782; Tomes et al. (1995) “Direct DNA Transfer into Intact Plant Cells via Microprojectile Bombardment, in Plant Cell, Tissue, and Organ Culture. Fundamental Methods, ed. Gamborg and Phillips (Springer-Verlag, Berlin); and McCabe et al. (1988) Biotechnology 6:923-926)); virus-mediated transformation (U.S. Pat. Nos. 5,889,191, 5,889,190, 5,866,785, 5,589,367 and 5,316,931); pollen transformation (De Wet et al. (1985) in The Experimental Manipulation of Ovule Tissues, ed. Chapman et al. (Longman, New York), pp. 197-209); Lec 1 transformation (U.S. patent application Ser. No. 09/435,054, WO 00/28058); whisker-mediated transformation (Kaeppler et al. (1990) Plant Cell Reports 9:415-418 and Kaeppler et al. (1992) Theor. Appl. Genet. 84:560-566); and chloroplast transformation technology (Bogorad, 2000, Trends in Biotechnology 18: 257-263; Ramesh et al., 2004, Methods Mol. Biol. 274:301-7; Hou et al., 2003, Transgenic Res. 12(1):111-4; Kindle et al., 1991, PNAS 88(5):1721-5; Bateman and Purton, 2000, Mol Gen Genet. 263(3):404-10; Sidorov et al., 1999, Plant J. 19(2):209-216)

The choice of transformation protocols used for generating transgenic plants and plant cells can vary depending on the type of plant or plant cell, i.e., monocot or dicot, targeted for transformation. Examples of transformation protocols particularly suited for a particular plant type include those for: onion (Weissinger et al. (1988) Ann. Rev. Genet. 22:421-477; Sanford et al. (1987) Particulate Science and Technology 5:27-37); potato (Tu et al. (1998) Plant Molecular Biology 37:829-838 and Chong et al. (2000) Transgenic Research 9:71-78); soybean (Christou et al. (1988) Plant Physiol. 87:671-674, McCabe et al. (1988) Bio/Technology 6:923-926, Finer and McMullen (1991) In Vitro Cell Dev. Biol. 27P:175-182, and Singh et al. (1998) Theor. Appl. Genet. 96:319-324); rice (Datta et al. (1990) Biotechnology 8:736-740, Li et al. (1993) Plant Cell Reports 12:250-255, and Christou and Ford (1995) Annals of Botany 75:407-413); maize (Klein et al. (1988) Proc. Natl. Acad. Sci. USA 85:4305-4309, Klein et al. (1988) Biotechnology 6:559-563, Klein et al. (1988) Plant Physiol. 91:440-444, Fromm et al. (1990) Biotechnology 8:833-839, and Tomes et al. (1995) “Direct DNA Transfer into Intact Plant Cells via Microprojectile Bombardment,” in Plant Cell, Tissue, and Organ Culture: Fundamental Methods, ed. Gamborg (Springer-Verlag, Berlin); cereals (Hooykaas-Van Slogteren et al. (1984) Nature (London) 311:763-764, U.S. Pat. No. 5,736,369); liliaceae (Bytebier et al. (1987) Proc. Natl. Acad. Sci. USA 84:5345-5349).

In some embodiments, more than one construct is used for transformation in the generation of transgenic plants and plant cells. Multiple constructs may be included in cis or trans positions. In preferred embodiments, each construct has a promoter and other regulatory sequences.

The cells that have been transformed may be grown into plants in accordance with any method known in the art (e.g., McCormick et al. (1986) Plant Cell Reports 5:81-84). These plants may then be grown, and either pollinated with the same transformed strain or different strains. Two or more generations of the plants may be grown to ensure that expression of the desired nucleic acid molecule, polypeptide and/or phenotypic characteristic is stably maintained and inherited.

### 5.7 Determination of Expression

Any method known in the art can be used for determining the level of expression in a plant of a nucleic acid molecule of the invention or polypeptide encoded therefrom. For example, the expression level in a plant of a polypeptide encoded by a nucleic acid molecule of the invention can be determined by immunoassay, quantitative gel electrophoresis, etc. Additionally, the expression level in a plant of a polypeptide encoded by a nucleic acid molecule of the invention can be determined by the degree to which the plant phenotype is altered. Determinations can be made using whole plants, tissues thereof, or plant cell culture.

In one embodiment, a comparison of polypeptide expression levels is made between a plant transformed with a nucleic acid molecule comprising one or more altered codons and a plant transformed with an unaltered nucleic acid molecule, wherein both nucleic acid molecule encode the same or substantially similar polypeptides. In another embodiment, a comparison of polypeptide expression levels is made between a plant transformed with a nucleic acid molecule comprising one or more altered codons and a non-transgenic plant.

The contents of all patents, patent applications, published PCT applications and articles, books, references, reference manuals and abstracts cited herein are hereby incorporated by reference in their entirety to more fully describe the state of the art to which the invention pertains.

As various changes may be made in the above-described subject matter without departing from the scope and spirit of the present invention, it is intended that all subject matter contained in the above description, or defined in the appended claims, be interpreted as descriptive and illustrative of the present invention. Many modifications and variations of the present invention are possible in light of the above teachings.

### 6. EXAMPLES

The following examples as set forth herein are meant to illustrate and exemplify the various aspects of carrying out the present invention and are not intended to limit the invention in any way.

### Example 1

### Design of Monocotyledonous Plant Virus Codon-Biased Nucleic Acid Molecule Coding Sequence Encoding Variants of the Bacillus thuringiensis Insecticidal Polypeptides 473N

Codons for nucleic acid molecules encoding the amino acid sequences of 473N were selected initially according to the 0.09-threshold monocotyledonous plant virus codon usage frequencies listed in Table 14, and subsequently Kozak consensus-optimized, and edited to eliminate cryptic splice sites, sequences that may cause rapid degradation of mRNA, spurious poly-adenylation signal sequences, and long alternate reading frames. In addition codons that have higher plant virus codon usage frequencies were positioned towards the 5′ end of the coding sequence. SEQ ID NO:1 encodes Kozak-473N. SEQ ID NO:2 is the amino acid sequence of Kozak-473N. Pre-codon optimized 473N is SEQ ID NO:15.

The following table indicates the codon usage frequencies of the monocotyledonous plant codon-biased nucleic acid molecule coding sequence listed as SEQ ID NO:1 compared to the monocotyledonous plant virus codon usage frequencies listed in Table 14.

TABLE 23

Codon usage frequencies in SEQ ID NO:1 compared to
monocotyledonous plant virus codon usage
frequencies adjusted with a cut-off threshold
greater than 0.09.

| | | | | | |
|---|---|---|---|---|---|
| | | | Virus | Codon | Codon |
| | | | >0.09 | optimized | optimized |
| | | | Threshold | 473R | 473R |
| Amino | | Codon | Adjusted | Codon | Codon |
| acid | Codon | Freq | Freq | Freq | Count |

| | | | | | |
|---|---|---|---|---|---|
| Ala | GCA | 0.31 | 0.31 | 0.28 | 9 |
| | GCC | 0.21 | 0.21 | 0.19 | 6 |
| | GCG | 0.14 | 0.14 | 0.12 | 4 |
| | GCT | 0.34 | 0.34 | 0.41 | 13 |
| Arg | AGA | 0.32 | 0.35 | 0.35 | 14 |
| | AGG | 0.17 | 0.18 | 0.17 | 7 |
| | CGA | 0.14 | 0.15 | 0.15 | 6 |
| | CGC | 0.14 | 0.15 | 0.15 | 6 |
| | CGG | 0.09 | 0 | 0 | 0 |
| | CGT | 0.16 | 0.17 | 0.17 | 7 |
| Asn | AAC | 0.42 | 0.42 | 0.5 | 35 |
| | AAT | 0.58 | 0.58 | 0.5 | 35 |
| Asp | GAC | 0.38 | 0.38 | 0.38 | 9 |
| | GAT | 0.62 | 0.62 | 0.62 | 15 |
| Cys | TGC | 0.44 | 0.44 | 0.33 | 1 |
| | TGT | 0.56 | 0.56 | 0.67 | 2 |
| Gln | CAA | 0.58 | 0.58 | 0.56 | 15 |
| | CAG | 0.42 | 0.42 | 0.44 | 12 |
| Glu | GAA | 0.6 | 0.6 | 0.61 | 14 |
| | GAG | 0.4 | 0.4 | 0.39 | 9 |
| Gly | GGA | 0.37 | 0.37 | 0.45 | 19 |
| | GGC | 0.2 | 0.2 | 0.21 | 9 |
| | GGG | 0.14 | 0.14 | 0.12 | 5 |
| | GGT | 0.28 | 0.28 | 0.21 | 9 |
| His | CAC | 0.43 | 0.43 | 0.46 | 6 |
| | CAT | 0.57 | 0.57 | 0.54 | 7 |
| Ile | ATA | 0.3 | 0.3 | 0.31 | 9 |
| | ATC | 0.29 | 0.29 | 0.31 | 9 |
| | ATT | 0.41 | 0.41 | 0.38 | 11 |
| Leu | CTA | 0.13 | 0.13 | 0.14 | 9 |
| | CTC | 0.14 | 0.14 | 0.15 | 10 |
| | CTG | 0.13 | 0.13 | 0.11 | 7 |
| | CTT | 0.18 | 0.18 | 0.21 | 14 |
| | TTA | 0.21 | 0.21 | 0.18 | 12 |
| | TTG | 0.21 | 0.21 | 0.21 | 14 |
| Lys | AAA | 0.53 | 0.53 | 0.3 | 3 |
| | AAG | 0.47 | 0.47 | 0.7 | 7 |
| Met | ATG | 1 | 1 | 1 | 9 |
| Phe | TTC | 0.46 | 0.46 | 0.69 | 25 |
| | TTT | 0.54 | 0.54 | 0.31 | 11 |
| Pro | CCA | 0.38 | 0.38 | 0.5 | 13 |
| | CCC | 0.17 | 0.17 | 0.04 | 1 |
| | CCG | 0.14 | 0.14 | 0.15 | 4 |
| | CCT | 0.31 | 0.31 | 0.31 8 | |
| STOP | TAA | 0.34 | 0.34 | 0 0 | |
| | TAG | 0.25 | 0.25 | 1 | 1 |
| | TGA | 0.41 | 0.41 | 0 | 0 |
| Ser | AGC | 0.13 | 0.13 | 0.12 | 7 |
| | AGT | 0.18 | 0.18 | 0.16 | 9 |
| | TCA | 0.24 | 0.24 | 0.25 | 14 |
| | TCC | 0.14 | 0.14 | 0.12 | 7 |
| | TCG | 0.1 | 0.1 | 0.11 | 6 |
| | TCT | 0.21 | 0.21 | 0.23 | 13 |
| Thr | ACA | 0.3 | 0.3 | 0.32 | 17 |
| | ACC | 0.2 | 0.2 | 0.21 | 11 |
| | ACG | 0.16 | 0.16 | 0.15 | 8 |
| | ACT | 0.34 | 0.34 | 0.32 | 17 |
| Trp | TGG | 1 | 1 | 1 | 7 |
| Tyr | TAC | 0.43 | 0.43 | 0.48 | 12 |
| | TAT | 0.57 | 0.57 | 0.52 | 13 |
| Val | GTA | 0.19 | 0.19 | 0.16 | 7 |
| | GTC | 0.21 | 0.21 | 0.23 | 10 |
| | GTG | 0.25 | 0.25 | 0.28 | 12 |
| | GTT | 0.36 | 0.36 | 0.33 | 14 |

### Example 2

### Assembly of Plant Virus Codon-Biased 473N

The synthetic version of the 473N gene (SEQ ID NO: 1) was synthesized by DNA2.0 (Menlo Park, Calif.). Restriction enzyme sites BamHI and HpaI were added to the 5′ and 3′ ends of the gene, respectively, to facilitate cloning into a transformation vector.

### Example 3

### Construction of a 473N Plant Transformation Vector

A 2.1 kb fragment corresponding to the 473N gene was isolated from the DNA2.0 vector after digestion of the plasmid with BamHI and HpaI. This fragment was subcloned into an intermediate vector, pSKNA-Ubi, using BamHI and HpaI resulting in pSKNA-Ubi:473N. pSKNA-Ubi:473N contains the 473N gene under the control of the maize Ubi promoter-5′UTR-Ubi intron 1 combination and is terminated by the pinII terminator sequence immediately 3′ to the 473N gene. pSKNA-Ubi:473N was digested with AscI and NotI to release the expression cassette (Ubi Pro-5′UTR′Ubi intron 1:473N:pinII), and this fragment was subcloned into the corresponding sites in the final transformation vector placing it upstream and in the opposite orientation to the selectable marker gene. The complete cassette between the LB and RB were sequence verified prior to transformation.

### Example 4

### Transformation of Maize by Particle Bombardment and Regeneration of Transgenic Plants

Immature maize embryos from greenhouse donor plants are bombarded with a DNA molecule containing a plant virus codon-biased nucleic acid molecule coding sequence operably linked to a ubiquitin promoter and a selectable marker gene such PAT (Wohlleben et al., 1988, Gene 70:25-37), which confers resistance to the herbicide Bialaphos. Alternatively, the selectable marker gene can be provided on a separate DNA molecule. Transformation is performed as follows. Media recipes follow below.

Preparation of Target Tissue

The ears are husked and surface sterilized in 30% Clorox™ bleach plus 0.5% Micro detergent for 20 minutes, and rinsed two times with sterile water. The immature embryos are excised and placed embryo axis side down (scutellum side up), 25 embryos per plate, on 560Y medium for 4 hours and then aligned within the 2.5-cm target zone in preparation for bombardment.

Preparation of DNA

A plasmid vector comprising the plant virus codon-biased nucleic acid molecule operably linked to a ubiquitin promoter is isolated. For example, a suitable transformation vector comprises a Ubi1 promoter from Zea mays, a 5′ UTR from Ubi1 and a Ubi1 intron, in combination with a PinII terminator. The vector additionally contains a selectable marker gene such as GAT driven by the maize Ubi1 promoter/inron/5′UTR with a 3×35S enhancer and a PinII terminator. Optionally, the selectable marker can reside on a separate plasmid. A DNA molecule comprising a plant virus codon-biased nucleic acid molecule coding sequence as well as a selectable marker such as GAT is precipitated onto 1.1 μm (average diameter) tungsten pellets using a CaCl₂ precipitation procedure as follows:
100 μl prepared tungsten particles in water
10 μl (1 μg) DNA in Tris EDTA buffer (1 μg total DNA)
100 μl 2.5 M CaCl₂
10 μl 0.1 M spermidine

Each reagent is added sequentially to a tungsten particle suspension, while maintained on the multitube vortexer. The final mixture is sonicated briefly and allowed to incubate under constant vortexing for 10 minutes. After the precipitation period, the tubes are centrifuged briefly, liquid removed, washed with 500 ml 100% ethanol, and centrifuged for 30 seconds. Again the liquid is removed, and 105 μl 100% ethanol is added to the final tungsten particle pellet. For particle gun bombardment, the tungsten/DNA particles are briefly sonicated and 10 μl spotted onto the center of each macrocarrier and allowed to dry about 2 minutes before bombardment.

Particle Gun Treatment

The sample plates are bombarded at level #4 in particle gun HE34-1 or HE34-2. All samples receive a single shot at 650 PSI, with a total often aliquots taken from each tube of prepared particles/DNA.

Following bombardment, the embryos are kept on 560Y medium for 2 days, then transferred to 560R selection medium containing 3 mg/liter 3 mM glyphosate, and subcultured every 2 weeks. After approximately 10 weeks of selection, selection-resistant callus clones are transferred to 288J medium to initiate plant regeneration. Following somatic embryo maturation (2-4 weeks), well-developed somatic embryos are transferred to medium for germination and transferred to the lighted culture room. Approximately 7-10 days later, developing plantlets are transferred to 272V hormone-free medium in tubes for 7-10 days until plantlets are well established. Plants are then transferred to inserts in flats (equivalent to 2.5″ pot) containing potting soil and grown for 1 week in a growth chamber, subsequently grown an additional 1-2 weeks in the greenhouse, then transferred to classic 600 pots (1.6 gallon) and grown to maturity. Plants are monitored and scored for expression of the polypeptide encoded by the plant virus codon-biased nucleic acid molecule by assays known in the art, such as, for example, immunoassays and western blotting with an antibody that binds to the encoded polypeptide. Polypeptide expression can also be monitored on resistant callus after 10 weeks of selection to evaluate levels of these polypeptides.

Bombardment and Culture Media

Bombardment medium (560Y) comprises 4.0 g/l N6 basal salts (SIGMA C-1416), 1.0 ml/l Eriksson's Vitamin Mix (1000×SIGMA-1511), 0.5 mg/l thiamine HCl, 120.0 g/l sucrose, 1.0 mg/l 2,4-D, and 2.88 g/l L-proline (brought to volume with dI H₂0 following adjustment to pH 5.8 with KOH); 2.0 g/l Gelrite™ (added after bringing to volume with dI H₂0); and 8.5 mg/l silver nitrate (added after sterilizing the medium and cooling to room temperature). Selection medium (560R) comprises 4.0 g/l N6 basal salts (SIGMA C-1416), 1.0 ml/l Eriksson's Vitamin Mix (1000×SIGMA-1511), 0.5 mg/l thiamine HCl, 30.0 g/l sucrose, and 2.0 mg/l 2,4-D (brought to volume with dl H₂0 following adjustment to pH 5.8 with KOH); 3.0 g/l Gelrite™ (added after bringing to volume with dI H₂0); and 0.85 mg/l silver nitrate and 3.0 mg/l Bialaphos (both added after sterilizing the medium and cooling to room temperature).

Plant regeneration medium (288J) comprises 4.3 g/l MS salts (GIBCO 11117-074), 5.0 ml/l MS vitamins stock solution (0.100 g nicotinic acid, 0.02 g/l thiamine HCl, 0.10 g/l pyridoxine HCl, and 0.40 g/l Glycine brought to volume with polished D-1 H₂0) (Murashige and Skoog (1962) Physiol. Plant. 15:473), 100 mg/l myo-inositol, 0.5 mg/l zeatin, 60 g/l sucrose, and 1.0 ml/l of 0.1 mM abscisic acid (brought to volume with polished dI H₂0 after adjusting to pH 5.6); 3.0 g/l Gelrite™ (added after bringing to volume with dI H₂0); and 1.0 mg/l indoleacetic acid and 3.0 mg/l Bialaphos (added after sterilizing the medium and cooling to 60° C.). Hormone-free medium (272V) comprises 4.3 g/l MS salts (GIBCO 11117-074), 5.0 ml/l MS vitamins stock solution (0.100 g/l nicotinic acid, 0.02 g/l thiamine HCl, 0.10 g/l pyridoxine HCl, and 0.40 g/l Glycine brought to volume with polished dI H₂O), 0.1 g/l myo-inositol, and 40.0 g/l sucrose (brought to volume with polished dI H₂0 after adjusting pH to 5.6); and 6 g/l Bacto-agar (added after bringing to volume with polished dl H₂0), sterilized and cooled to 60° C.

### Example 5

### Agrobacterium-Mediated Transformation of Maize and Regeneration of Transgenic Plants

Transformation of maize with a vector containing a plant virus codon-bias 473N gene was performed by the method of Zhao (U.S. Pat. No. 5,981,840 and PCT patent publication WO98/32326; the contents of each of which are hereby incorporated by reference).

Agrobacterium were grown on a master plate of 800 medium and cultured at 28° C. in the dark for 3 days, and thereafter stored at 4° C. for up to one month. Working plates of Agrobacterium were grown on 810 medium plates and incubated in the dark at 28° C. for one to two days.

Briefly, embryos were dissected from fresh, sterilized corn ears and kept in 561Q medium until all required embryos were collected. Embryos were then contacted with an Agrobacterium suspension prepared from the working plate, in which the Agrobacterium contained a plasmid comprising the 473N gene of the embodiments. The embryos were co-cultivated with the Agrobacterium on 562P plates, with the embryos placed axis down on the plates, as per the '840 patent protocol.

After one week on 562P medium, the embryos were transferred to 563O medium. The embryos were subcultured on fresh 563O medium at 2 week intervals and incubation was continued under the same conditions. Callus events began to appear after 6 to 8 weeks on selection.

After the calli have reached the appropriate size, the calli were cultured on regeneration (288W) medium and kept in the dark for 2-3 weeks to initiate plant regeneration. Following somatic embryo maturation, well-developed somatic embryos were transferred to medium for germination (272V) and transferred to a lighted culture room. Approximately 7-10 days later, developing plantlets were transferred to 272V hormone-free medium in tubes for 7-10 days until plantlets were well established. Plants were then transferred to inserts in flats (equivalent to 2.5″ pot) containing potting soil and grown for 1 week in a growth chamber, subsequently grown an additional 1-2 weeks in the greenhouse, then transferred to classic 600 pots (1.6 gallon) and grown to maturity.

Media used in Agrobacterium-mediated transformation and regeneration of transgenic maize plants:

561O medium comprises 4.0 g/L N6 basal salts (SIGMA C-1416), 1.0 mL/L Eriksson's Vitamin Mix (1000×SIGMA-1511), 0.5 mg/L thiamine HCl, 68.5 g/L sucrose, 36.0 g/L glucose, 1.5 mg/L 2,4-D, and 0.69 g/L L-proline (brought to volume with dI H₂O following adjustment to pH 5.2 with KOH); 2.0 g/L Gelrite™ (added after bringing to volume with dI H₂O); and 8.5 mg/L silver nitrate (added after sterilizing the medium and cooling to room temperature).

800 medium comprises 50.0 mL/L stock solution A and 850 mL dI H₂O, and brought to volume minus 100 mL/L with dI H₂O, after which is added 9.0 g of phytagar. After sterilizing and cooling, 50.0 mL/L stock solution B is added, along with 5.0 g of glucose and 2.0 mL of a 50 mg/mL stock solution of spectinomycin. Stock solution A comprises 60.0 g of dibasic K₂HPO₄ and 20.0 g of monobasic sodium phosphate, dissolved in 950 mL of water, adjusted to pH 7.0 with KOH, and brought to 1.0 L volume with dI H₂O. Stock solution B comprises 20.0 g NH₄Cl, 6.0 g MgSO₄.7H₂O, 3.0 g potassium chloride, 0.2 g CaCl₂, and 0.05 g of FeSO₄.7H₂O, all brought to volume with dI H₂O, sterilized, and cooled.

810 medium comprises 5.0 g yeast extract (Difco), 10.0 g peptone (Difco), 5.0 g NaCl, dissolved in dI H₂O, and brought to volume after adjusting pH to 6.8. 15.0 g of bacto-agar is then added, the solution is sterilized and cooled, and 1.0 mL of a 50 mg/mL stock solution of spectinomycin is added.

562P medium comprises 4.0 g/L N6 basal salts (SIGMA C-1416), 1.0 mL/L Eriksson's Vitamin Mix (1000×SIGMA-1511), 0.5 mg/L thiamine HCl, 30.0 g/L sucrose, and 2.0 mg/L 2,4-D (brought to volume with dI H₂0 following adjustment to pH 5.8 with KOH); 3.0 g/L Gelrite™ (added after bringing to volume with dI H₂0); and 0.85 mg/L silver nitrate and 1.0 mL of a 100 mM stock of acetosyringone (both added after sterilizing the medium and cooling to room temperature).

563O medium comprises 4.0 g/L N6 basal salts (SIGMA C-1416), 1.0 mL/L Eriksson's Vitamin Mix (1000×SIGMA-1511), 0.5 mg/L thiamine HCl, 30.0 g/L sucrose, 1.5 mg/L 2,4-D, 0.69 g L-proline, and 0.5 g MES buffer (brought to volume with dI H₂0 following adjustment to pH 5.8 with KOH). Then, 6.0 g/L Ultrapure™ agar-agar (EM Science) is added and the medium is sterilized and cooled. Subsequently, 0.85 mg/L silver nitrate, 3.0 mL of a 1 mg/mL stock of Bialaphos, and 2.0 mL of a 50 mg/mL stock of carbenicillin are added.

288 W medium comprises 4.3 g/L MS salts (GIBCO 11117-074), 5.0 mL/L MS vitamins stock solution (0.100 g nicotinic acid, 0.02 g/L thiamine HCl, 0.10 g/L pyridoxine HCl, and 0.40 g/L Glycine brought to volume with polished D-I H₂0) (Murashige and Skoog (1962) Physiol. Plant. 15:473), 100 mg/L myo-inositol, 0.5 mg/L zeatin, and 60 g/L sucrose, which is then brought to volume with polished D-I H₂0 after adjusting to pH 5.6. Following, 6.0 g/L of Ultrapure™ agar-agar (EM Science) is added and the medium is sterilized and cooled. Subsequently, 1.0 mL/L of 0.1 mM abscisic acid; 1.0 mg/L indoleacetic acid and 3.0 mg/L Bialaphos are added, along with 2.0 mL of a 50 mg/mL stock of carbenicillin.

Hormone-free medium (272V) comprises 4.3 g/L MS salts (GIBCO 11117-074), 5.0 mL/L MS vitamins stock solution (0.100 g/L nicotinic acid, 0.02 g/L thiamine HCl, 0.10 g/L pyridoxine HCl, and 0.40 g/L Glycine brought to volume with polished dI H20), 0.1 g/L myo-inositol, and 40.0 g/L sucrose (brought to volume with polished dI H20 after adjusting pH to 5.6); and 6 g/L Bacto-agar (added after bringing to volume with polished dI H20), sterilized and cooled to 60° C.

### Example 6

### Insect Bioassay of Transgenic 473N Expressing Calli

Insects were bioassayed on transgenic calli expressing 473N under the Ubiquitin promoter to determine whether there was sufficient expression of 473N toxin at this stage to provide insecticidal activity. This assay in combination with the western blot analysis provided a measure of how well the plant virus codon-biased 473N gene, encoding an insecticidal polypeptide, was expressed in plant tissues.

The callus assay was performed in Pitman trays that were previously sterilized by 95% ethanol spray. Agar (Serva) prepared according to the manufacturer's instructions and supplemented with a triple antibiotic solution (70 mls/500 ml agar) containing penicillin, streptomycin and amphotercin B was poured into each well and allowed to cool. A sterile filter paper disc was placed on top of the agar in each well and 200 μl of sterile water dispensed onto the filter paper. Callus (˜1 cm in size) was added onto the filter paper and 2 European corn borer (ECB) neonates were added per well. The assay plates were incubated at 27° C. and insects were scored for mortality, stunting of growth, and behavioral changes at 72-96 h after insect addition. The assay was repeated twice to confirm scores.

The results of the assays showed that neonate ECB were either severely stunted or dead in 30% of the wells tested. Correlation of activity between the two repetitions was 100%. No mortality or stunting was observed in non-transgenic control callus. This test indicated that 473N was expressed at insecticidal amounts in a proportion of the different callus and supported the effectiveness of the plant virus codon bias.

### Example 7

### Leaf Disc Efficacy Testing of ECB and CEW

Transformed calli were regenerated into plants and sent to the greenhouse for T0 efficacy testing with ECB and corn earworm (CEW). Leaf disc assays were performed on all events at the V6 developmental stage to evaluate plant protection based on the area of leaf consumed by neonate insect after 48 hrs. Assays were conducted by punching multiple leaf discs for each transgenic event tested and placing one disc per well of a 24 well plate. Four leaf discs per event per insect (8 total) were used in the assay. The leaf discs were maintained on a moist filter paper disc that was the same diameter as the well. Lids were placed on each plate after addition of the insects to prevent them from escaping the well. Control leaf discs from non-transgenic plants were included for comparison of leaf consumption. Assays were conducted at 27° C.

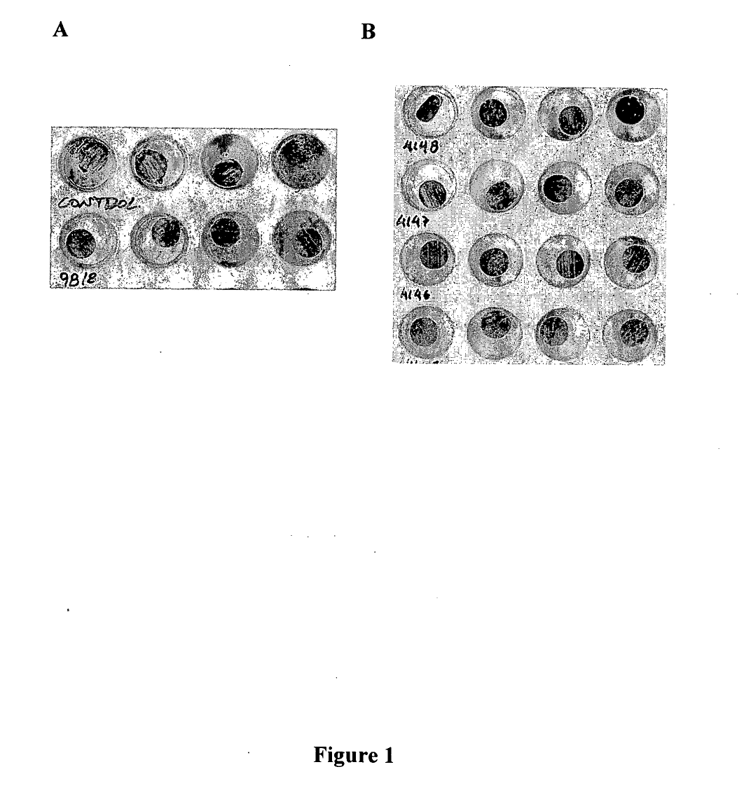

The results of this assay are summarized in Table 24 below. Leaf protection was observed in 45% of the events tested in the assay. Events that demonstrated protection against ECB also showed protection against CEW. The leaf disc was totally consumed in control wells and in the other “non-efficacious” events. An example of the leaf disc assay is shown in FIG. 1. These results support the ability to express a 473N gene at insecticidal levels that has been designed with a plant virus codon bias.

TABLE 24
Leaf disc assay results for events expressing a plant
virus codon-optimized 473N gene.

| Construct | ECB positive | CEW positive |
|---|---|---|
| PHP25637 | 21/47 | 21/41 |

### Example 8

### Immunoblot Analysis of Leaf Samples from 473N Transgenic Events

Plant polypeptide extractions were performed by collecting 4 leaf discs (˜100 mg) from V6 staged plants into a 1.2 ml raptor tube. For each sample two steel grinding balls and 200 μl of extraction buffer (100 mM potassium phosphate, pH 7.8, 1 mM EDTA, 10% glycerol, 1% Triton, 7 mM beta mercaptoethanol (BME) and protease inhibitor cocktail) was added. The tubes were capped and placed in a Geno/Grinder (BT&C/OPS Diagnostics, New Bridgewater, N.J.) and rapetted twice at a speed of 1650 for 30 sec. The samples were centrifuged at 4000 rpm for 15 minutes at 4° C., the supernatant transferred to a new tube and recentrifuged at 13,000 rpm for 5 min at 4° C. The supernatant was transferred to a new tube and the samples stored at −20° C. until use.

Samples were prepared for SDS-PAGE gel electrophoresis by adding 5 μl of 4× loading buffer (Invitrogen, Carlsbad, Calif.) and 3.5 μl of BME and heating at 1001C for 5 minutes. Samples are loaded onto a 4-16% NuPAGE precast gel (Invitrogen) with appropriate molecular weight markers and run at ˜125 volts for ˜90 minutes in MES running buffer.

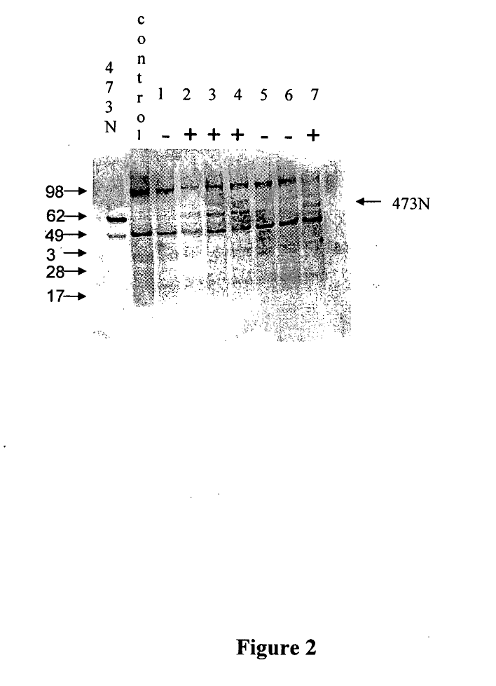

Immunoblot analysis was performed by removing the gel from the caster and placing into a blotting sandwich consisting of 2 sponge layers, blotting paper (cut to the size of the gel), the gel, the pre-wetted membrane, blotting paper, and two sponges. The sandwich was placed in the transfer box containing transfer buffer and run at 30 volts for 60 to 90 minutes. After transfer the membrane was removed from the sandwich and placed in a container to which 1×PBST (10 mM Phosphate buffered saline, pH7.4, 1% Tween 20) supplemented with 5% nonfat dry milk was added. Blocking was done for 1 h at RT with gentle agitation. After 1 h the blocking solution was replaced with 15 ml of 1×PBST+5% dry milk containing the proper dilution of primary 473N antibody and incubated with gentle shaking at 4° C. overnight. After incubation, the primary antibody was removed and the membrane washed 3 times (5 minutes each) with 1×PBST+5% dry milk. The membrane was incubated with secondary antibody at a 1/5000 dilution in 25 ml of 1×PBST+5% dry milk for 1 h at RT with gentle shaking. The secondary Ab was removed from the membrane and the membrane washed 3 times (5 min each) with 1×PBST+5% dry milk followed by 3 washes (5 min. each) of 1× Assay buffer (supplied in Western Light Kit™, Applied Biosystems, Foster City, Calif.). Excess buffer was drained away from the membrane and the membrane placed on plastic wrap to which 3 ml of substrate solution (CSPD™—provided in kit) supplemented with 150 μl of Nitro-Block II™ enhancer (provided in kit) was added for 5 min in the dark. The membrane was developed by draining away excess solution and exposing the membrane to Biomax Light X-ray film (Eastman Kodak Co. New Haven, Conn.) for different exposure times. The film was then developed by traditional methods. Western analysis of leaf tissue from 473N transgenic events showed an immunoreactive band to the Ab that was similar in size to the purified 473N protein control (see FIG. 2). The presence of this band was in leaf samples from events that demonstrated efficacy in the leaf disc assay further supporting the expression of a plant virus codon optimized 473N gene at insecticidal levels. This band was absent from non transgenic controls. Other cross reactive bands are in common between transgenic samples and non transgenic controls.

### Example 9

### Design of Monocotyledonous Plant Virus Codon-Biased Nucleic Acid Molecule Coding Sequence Encoding an Insecticidal Lipase from Rhyzopus oryzae (RoLipase)

Codons for nucleic acid molecule encoding the amino acid sequences of RoLipase with a Barley Alpha Amylase signal peptide were selected initially according to the 0.09-threshold monocotyledonous plant virus codon usage frequencies listed in Table 14. Subsequently the sequence was Kozak consensus-optimized and edited to eliminate cryptic splice sites, sequences that may cause rapid degradation of mRNA, spurious poly-adenylation signal sequences, and long alternate reading frames. In addition codons that have higher plant virus codon usage frequencies were positioned towards the 5′ end of the coding sequence. SEQ ID NO:3 encodes codon optimized RoLipase. SEQ ID NO:4 is the amino acid sequence of codon optimized RoLipase. SEQ ID NOS:5 and 6 is the a Barley Alpha Amylase signal peptide (nucleic acid and peptide sequence, respectively) that was added to the codon optimized RoLipase sequence and used for all experiments described. Pre-codon optimized lipase is SEQ ID NO:16 (also Genebank Accession No. AF229435).

### Example 10

### Assembly of Plant Virus Codon-Biased BAA-RoLipase

The synthetic version of the RoLipase (SEQ ID NO:3) with the was synthesized by DNA2.0 (Menlo Park, Calif.). Restriction enzyme sites BamHI and HpaI were added to the 5′ and 3′ ends of the gene, respectively, to facilitate cloning into a plant transformation vector.

### Example 11

### Construction of a BAA-RoLipase Plant Transformation Vector

A 1.2 kb fragment corresponding to the BAA-RoLipase gene was isolated from the supplied DNA2.0 vector after digestion of the plasmid with BamHI and HpaI. This fragment was subcloned into an intermediate vector, pSKNA-Ubi, using BamHI and HpaI resulting in pSKNA-Ubi:BAA-RoLipase. pSKNA-Ubi:BAA-RoLipase contained the BAA-RoLipase gene under the control of the maize Ubi promoter-5′UTR-Ubi intron 1 combination and was terminated by the pin II terminator sequence immediately 3′ to the Lipase gene. pSKNA-Ubi:BAA-RoLipase was digested with AscI and NotI to release the expression cassette (Ubi Pro-5′UTR′Ubi intron 1:BAA-RoLipase:pinII) and this fragment was subcloned into the corresponding sites in the final transformation vector placing it upstream and in the opposite orientation to the selectable marker gene. The complete cassette between the LB and RB were sequence verified prior to transformation.

The BAA-RoLipase plant transformation vector was used to transform maize by Agrobacterium-mediated transformation and plants were regenerated according to the procedures detailed in Example 5.

### Example 12

### Corn Rootworm Assay (CRW) on RoLipase Transformed Events

CRW evaluation was performed on 45 Rolipase transformed events using a root trainer assay. Rolipase plantlets from transformation were transplanted into root trainers and plants were infested at the V3-V4 stage with 100 CRW eggs. Plants were scored for root damage at 15-17 days post infestation and passed on the basis of root scores compared to non transgenic control plants. Eleven plants were scored as positive based on the degree of root damage representing a 24% keep rate (Table 25). A subset of these plants were selected for Western analysis of Rolipase expression.

TABLE 25
Rolipase T0 events that passed the CRW assay

| | | Percentage |
|---|---|---|
| Total Events Evaluated | No. of Events Passed | of Kept Events |
| 45 | 11 | 24 |

### Example 13

### Immunoblot Analysis of Leaf and Root Samples from BAA-RoLipase Transgenic Events

Plant polypeptide extractions were performed by collecting root and leaf sections (˜100 mg) from V6-8 staged plants into a 1.2 ml raptor tube. For each sample two steel grinding balls and 200 μl of extraction buffer (100 mM potassium phosphate, pH 7.8, 1 mM EDTA, 10% glycerol, 1% Triton, 7 mM beta mercaptoethanol (BME) and protease inhibitor cocktail) was added. The tubes were capped and placed in a Geno/Grinder (BT&C/OPS Diagnostics, New Bridgewater, N.J.) and rapetted twice at a speed of 1650 for 30 sec. The samples were centrifuged at 4000 rpm for 15 minutes at 4° C., the supernatant transferred to a new tube and recentrifuged at 13,000 rpm for 5 min at 4° C. The supernatant was transferred to a new tube and the samples stored at −20° C. until use.

Samples were prepared for SDS-PAGE gel electrophoresis by adding 5 μl of 4× loading buffer (Invitrogen, Carlsbad, Calif.) and 3.5 μl of BME and heating at 100° C. for 5 minutes. Samples are loaded onto a 4-16% NuPAGE precast gel (Invitrogen) with appropriate molecular weight markers and run at ˜125 volts for ˜90 minutes in MES running buffer.

Immunoblot analysis was performed by removing the gel from the caster and placing into a blotting sandwich consisting of 2 sponge layers, blotting paper (cut to the size of the gel), the gel, the pre-wetted membrane, blotting paper, and two sponges. The sandwich was placed in the transfer box containing transfer buffer and run at 30 volts for 60 to 90 minutes. After transfer the membrane was removed from the sandwich and placed in a container to which 1×PBST (10 mM Phosphate buffered saline, pH7.4, 1% Tween 20) supplemented with 5% nonfat dry milk was added. Blocking was done for 1 h at RT with gentle agitation. After 1 h the blocking solution was replaced with 15 ml of 1×PBST+5% dry milk containing a 1:1000 dilution of primary RoLipase antibody and incubated with gentle shaking at 4° C. overnight. After incubation, the primary antibody was removed and the membrane washed 3 times (5 minutes each) with 1×PBST+5% dry milk. The membrane was incubated with secondary antibody at a 1:5000 dilution in 25 ml of 1×PBST+5% dry milk for 1 h at RT with gentle shaking. The secondary Ab was removed from the membrane and the membrane washed 3 times (5 min each) with 1×PBST+5% dry milk followed by 3 washes (5 min. each) of 1× Assay buffer (supplied in Western Light Kit™, Applied Biosystems, Foster City, Calif.). Excess buffer was drained away from the membrane and the membrane placed on plastic wrap to which 3 ml of substrate solution (CSPD™—provided in kit) supplemented with 150 μl of Nitro-Block II™ enhancer (provided in kit) was added for 5 min in the dark. The membrane was developed by draining away excess solution and exposing the membrane to Biomax Light X-ray film (Eastman Kodak Co. New Haven, Conn.) for different exposure times. The film was then developed by traditional methods.

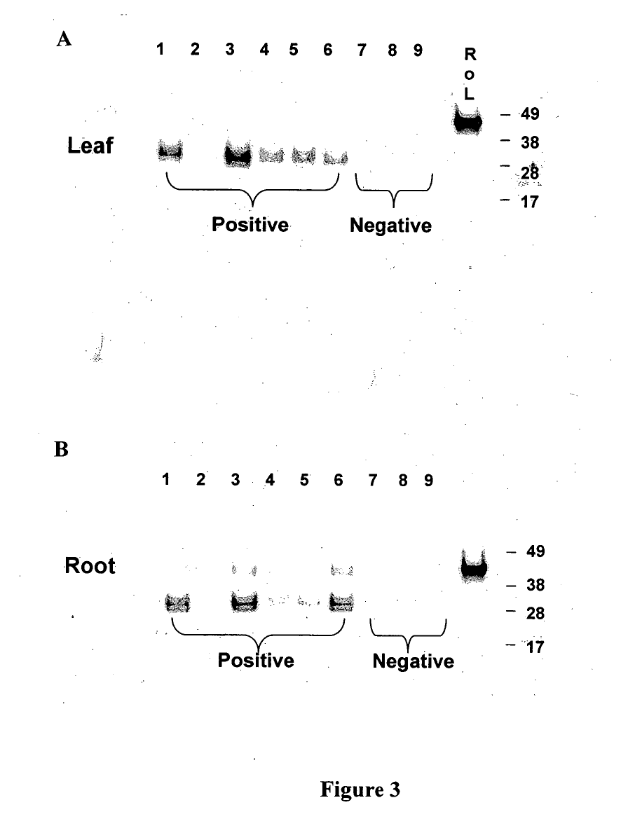

Western analysis of leaf and root tissue was performed on a subset of RoLipase transgenic events that were positive or negative in the root trainer assays. The results of these analyses showed an immunoreactive band corresponding to the expected size of mature Rolipase (˜31 kD) in events that were positive in the assay (see FIG. 3). A purified Rolipase precursor protein (ROL˜42 kD) was included in the Western analysis as a positive control. The correlation between root protection and the presence of the mature form of Rolipase in the tested events supports the successful expression of a plant virus codon optimized RoLipase gene.

What is claimed:

1. A method of designing a nucleic acid molecule encoding a polypeptide for expression of said polypeptide in a plant comprising altering at least one codon of a nucleic acid molecule to an altered codon, wherein said altered codon is selected from a group consisting of codons having a usage frequency in one or more plant viruses that is greater than that of said codon of said nucleic acid molecule.

2. The method of claim 1, wherein said altered codon has a usage frequency in one or more plant viruses that is greater than 0.09.

3. The method of claim 1, wherein said altered codon has a usage frequency in one or more plant viruses that is equal to or greater than the median codon usage frequency for an amino acid encoded by said altered codon in said one or more plant viruses, wherein said median codon usage frequency is the median of the codon usage frequencies in one or more plant viruses for all codons encoding said amino acid.

4. The method of claim 1, wherein at least 30% of codons in said nucleic acid molecule comprising at least one altered codon are altered codons.

5. The method of claim 1, wherein an equal or greater number of altered codons exist in a first portion of a nucleic acid molecule comprising at least one altered codon than in a second portion of said nucleic acid molecule, wherein said first portion is 5′ to said second portion.

6. The method of claim 5, wherein said first portion consists of one third of said nucleic acid molecule and said second portion consists of two thirds of said nucleic acid molecule.

7. The method of claim 5, wherein said first portion consists of one quarter of said nucleic acid molecule and said second portion consists of three quarters of said nucleic acid molecule.

8. The method of claim 5, wherein said first and second portions of said nucleic acid molecule are equal in length and said first portion has a greater number of said altered codons.

9. The method of claim 1, wherein expression of said polypeptide in a plant encoded by a nucleic acid molecule comprising at least one altered codon causes a change in a phenotype of said plant as compared to a plant not expressing said polypeptide.

10. The method of claim 1, wherein expression of a nucleic acid molecule comprising at least one altered codon causes a change in a phenotype of said plant as compared to a plant expressing a nucleic acid molecule that does not comprise at least one altered codon, wherein said nucleic acid molecules encode the same polypeptide.

11. The method of claim 9 or 10, wherein said phenotype is selected from the group consisting of insect resistance, insect tolerance, disease resistance, disease tolerance, nematode resistance, nematode tolerance, drought tolerance, salt tolerance, heavy metal tolerance, heavy metal detoxification, low phytate content, high-efficiency nitrogen usage, yield enhancement, increased yield stability, improved nutritional content, increased sugar content, improved growth and vigor, improved digestibility, expression of therapeutic polypeptides, synthesis of non-polypeptide pharmaceuticals, resistance to a selection agent, fluorescence, luminescence, recombinase activity, and male sterility.

12. The method of claim 9 or 10, wherein said phenotype is increased expression of said polypeptide in said plant.

13. The method of claim 1, wherein said plant is a monocotyledonous plant.

14. The method of claim 13, wherein said monocotyledonous plant is selected from the group consisting of barley, maize, millet, oats, rice, and wheat.

15. The method of claim 14, wherein said monocotyledonous plant is maize.

16. The method of claim 1, wherein said plant is a dicotyledonous plant.

17. The method according to claim 16, wherein said dicotyledonous plant is selected from the group consisting of potato, soybean, tobacco, and tomato.

18. The method of claim 17, wherein said dicotyledonous plant is soybean.

19. The method of claim 1 or 13, wherein said one or more plant viruses are monocotyledonous plant viruses.

20. The method of claim 19, wherein said at least one codon encodes
a) alanine and said altered codon is selected from the group consisting of GCA and GCT;
b) arginine and said altered codon is selected from the group consisting of AGA, AGG, and CGT;
c) asparagine and said altered codon is AAT;
d) aspartic acid and said altered codon is GAT;
e) cysteine and said altered codon is TGT;
f) glutamine and said altered codon is CAA;
g) glutamic acid and said altered codon is GAA;
h) glycine and said altered codon is selected from the group consisting of GGA and GGT;
i) histidine and said altered codon is CAT;
j) isoleucine and said altered codon is selected from the group consisting of ATA and ATT;
k) leucine and said altered codon is selected from the group consisting of CTT, TTA, and TTG;
l) lysine and said altered codon is AAA;
m) phenylalanine and said altered codon is TTT;
n) proline and said altered codon is selected from the group consisting of CCA and CCT;
o) serine and said altered codon is selected from the group consisting of AGT, TCA, and TCT;
p) threonine and said altered codon is selected from the group consisting of ACA and ACT;
q) tyrosine and said altered codon is TAT; or
r) valine and said altered codon is selected from the group consisting of GTG and GTT.

21. The method of claim 19, wherein said one or more monocotyledonous plant viruses is a maize-specific virus.

22. The method of claim 21, wherein said at least one codon encodes
a) alanine and said altered codon is selected from the group consisting of GCA and GCC;
b) arginine and said altered codon is selected from the group consisting of AGA, AGG, and CGC;
c) asparagine and said altered codon is AAT;
d) aspartic acid and said altered codon is GAT;
e) cysteine and said altered codon is TGT;
f) glutamine and said altered codon is selected from the group consisting of CAA and CAG;
g) glutamic acid and said altered codon is GAA;
h) glycine and said altered codon is selected from the group consisting of GGA and GGT;
i) histidine and said altered codon is CAT;
j) isoleucine and said altered codon is selected from the group consisting of ATC and ATT;
k) leucine and said altered codon is selected from the group consisting of CTT, CTC, and TTG;
l) lysine and said altered codon is AAG;
m) phenylalanine and said altered codon is TTC;
n) proline and said altered codon is selected from the group consisting of CCA and CCT;
o) serine and said altered codon is selected from the group consisting of TCC, TCA, and TCT;
p) threonine and said altered codon is selected from the group consisting of ACA and ACT;
q) tyrosine and said altered codon is TAT; or
r) valine and said altered codon is selected from the group consisting of GTG and GTT.

23. The method of claim 21, wherein said usage frequency in one or more plant viruses is based on nucleic acid molecules encoding maize virus coat polypeptides and capsid polypeptides.

24. The method of claim 23, wherein said at least one codon encodes
a) alanine and said altered codon is selected from the group consisting of GCA and GCT;
b) arginine and said altered codon is selected from the group consisting of AGA, AGG, and CGA;
c) asparagine and said altered codon is AAC;
d) aspartic acid and said altered codon is GAT;
e) cysteine and said altered codon is TGC;
f) glutamine and said altered codon is CAA;
g) glutamic acid and said altered codon is GAG;
h) glycine and said altered codon is selected from the group consisting of GGA and GGG;
i) histidine and said altered codon is CAT;
j) isoleucine and said altered codon is selected from the group consisting of ATC and ATT;
k) leucine and said altered codon is selected from the group consisting of CTG, CTC, and TTG;
l) lysine and said altered codon is AAG;
m) phenylalanine and said altered codon is TTC;
n) proline and said altered codon is selected from the group consisting of CCA and CCT;
o) serine and said altered codon is selected from the group consisting of TCC, TCA, and AGC;
p) threonine and said altered codon is selected from the group consisting of ACA and ACT;
q) tyrosine and said altered codon is TAT; or
r) valine and said altered codon is selected from the group consisting of GTC, GTG, and GTT.

25. The method of claim 1 or 16, wherein said one or more plant viruses are dicotyledonous plant viruses.

26. The method of claim 25, wherein said one or more diocotyledonous plant viruses is a soybean-specific virus.

27. The method of claim 25, wherein said usage frequency in one or more plant viruses is based on nucleic acid molecules encoding dicotyledonous plant virus coat polypeptides and capsid polypeptides.

28. The method of claim 27, wherein said at least one codon encodes
a) alanine and said altered codon is selected from the group consisting of GCC and GCT;
b) arginine and said altered codon is selected from the group consisting of AGA, AGG, and CGT;
c) asparagine and said altered codon is AAT;
d) aspartic acid and said altered codon is GAT;
e) cysteine and said altered codon is TGT;
f) glutamine and said altered codon is CAA;
g) glutamic acid and said altered codon is GAA;
h) glycine and said altered codon is selected from the group consisting of GGA and GGT;
i) histidine and said altered codon is CAT;
j) isoleucine and said altered codon is selected from the group consisting of ATA and ATT;
k) leucine and said altered codon is selected from the group consisting of CTT, TTA, and TTG;
l) lysine and said altered codon is AAG;
m) phenylalanine and said altered codon is TTT;
n) proline and said altered codon is selected from the group consisting of CCA, CCC, and CCT;
o) serine and said altered codon is selected from the group consisting of AGT, TCA, and TCT;
p) threonine and said altered codon is selected from the group consisting of ACA, ACC, and ACT;
q) tyrosine and said altered codon is TAT; or
r) valine and said altered codon is selected from the group consisting of GTG and GTT.

29. The method of claim 1, wherein a nucleic acid molecule comprising at least one altered codon has a codon usage frequency for all amino acid residues of at least one type of amino acid that is the same or substantially similar to the usage frequency in one or more plant viruses.

30. The method of claim 29, where in said one or more plant viruses are monocotyledonous plant viruses.

31. The method of claim 30, wherein said type of amino acid is
a) alanine and said codon usage frequency is GCA (0.31), GCC (0.21), GCG (0.14), and GCT (0.34);
b) arginine and said codon usage frequency is AGA (0.32), AGG (0.17), CGA (0.14), CGC (0.14), CGG (0.09), and CGT (0.16);
c) asparagine and said codon usage frequency is AAC (0.42) and AAT (0.58);
d) aspartic acid and said codon usage frequency is GAC (0.38) and GAT (0.62);
e) cysteine and said codon usage frequency is TGC (0.44) and TGT (0.56);
f) glutamine and said codon usage frequency is CAA (0.58) and CAG (0.42);
g) glutamic acid and said codon usage frequency is GAA (0.60) and GAG (0.40);
h) glycine and said codon usage frequency is GGA (0.37), GGC (0.20), GGG (0.14), and GGT (0.28);
i) histidine and said codon usage frequency is CAC (0.43) and CAT (0.57);
j) isoleucine and said codon usage frequency is ATA (0.30), ATC (0.29), and ATT (0.41);
k) leucine and said codon usage frequency is CTA (0.13), CTC (0.14), CTG (0.13), CTT (0.18), TTA (0.21), and TTG (0.21);
l) lysine and said codon usage frequency is AAA (0.53) and AAG (0.47);
m) phenylalanine and said codon usage frequency is TTC (0.46) and TTT (0.54);
n) proline and said codon usage frequency is CCA (0.38), CCC (0.17), CCG (0.14), and CCT (0.31);
o) serine and said codon usage frequency is AGC (0.13), AGT (0.18), TCA (0.24), TCC (0.14), TCG (0.10), and TCT (0.21);
p) threonine and said codon usage frequency is ACA (0.30), ACC (0.20), ACG (0.16), and ACT (0.34);
q) tyrosine and said codon usage frequency is TAC (0.43) and TAT (0.57); or
r) valine and said codon usage frequency is GTA (0.19), GTC (0.21), GTG (0.25), and GTT (0.36).

32. The method of claim 30, wherein said monocotyledonous plant viruses are maize-specific viruses.

33. The method of claim 32, wherein said type of amino acid is
a) alanine and said codon usage frequency is GCA (0.31), GCC (0.30), GCG (0.11), and GCT (0.28);
b) arginine and said codon usage frequency is AGA (0.27), AGG (0.17), CGA (0.12), CGC (0.19), CGG (0.12), and CGT (0.13);
c) asparagine and said codon usage frequency is AAC (0.44) and AAT (0.56);
d) aspartic acid and said codon usage frequency is GAC (0.41) and GAT (0.59);
e) cysteine and said codon usage frequency is TGC (0.42) and TGT (0.58);
f) glutamine and said codon usage frequency is CAA (0.50) and CAG (0.50);
g) glutamic acid and said codon usage frequency is GAA (0.52) and GAG (0.48);
h) glycine and said codon usage frequency is GGA (0.36), GGC (0.23), GGG (0.17), and GGT (0.24);
i) histidine and said codon usage frequency is CAC (0.45), CAT (0.55);
j) isoleucine and said codon usage frequency is ATA (0.27), ATC (0.30), and ATT (0.43);
k) leucine and said codon usage frequency is CTA (0.12), CTC (0.22), CTG (0.16), CTT (0.19), TTA (0.14), and TTG (0.18);
l) lysine and said codon usage frequency is AAA (0.49) and AAG (0.51);
m) phenylalanine and said codon usage frequency is TTC (0.56) and TTT (0.44);
n) proline and said codon usage frequency is CCA (0.31), CCC (0.20), CCG (0.17), and CCT (0.32);
o) serine and said codon usage frequency is AGC (0.12), AGT (0.12), TCA (0.22), TCC (0.21), TCG (0.10), and TCT (0.22);
p) threonine and said codon usage frequency is ACA (0.32), ACC (0.26), ACG (0.13), and ACT (0.29);
q) tyrosine and said codon usage frequency is TAC (0.46) and TAT (0.54); or
r) valine and said codon usage frequency is GTA (0.16), GTC (0.25), GTG (0.26), and GTT (0.33).

34. The method of claim 32, wherein said usage frequency in one or more plant viruses is based on nucleic acid molecules encoding maize virus coat polypeptide and capsid polypeptide.

35. The method of claim 32, wherein said type of amino acid is
a) alanine and said codon usage frequency is GCA (0.38), GCC (0.22), GCG (0.14), and GCT (0.26);
b) arginine and said codon usage frequency is AGA (0.30), AGG (0.18), CGA (0.18), CGC (0.16), CGG (0.11), and CGT (0.07);
c) asparagine and said codon usage frequency is AAC (0.53) and AAT (0.47);
d) aspartic acid and said codon usage frequency is GAC (0.45) and GAT (0.55);
e) cysteine and said codon usage frequency is TGC (0.53) and TGT (0.47);
f) glutamine and said codon usage frequency is CAA (0.52) and CAG (0.48);
g) glutamic acid and said codon usage frequency is GAA (0.44) and GAG (0.56);
h) glycine and said codon usage frequency is GGA (0.42), GGC (0.18), GGG (0.23), and GGT (0.18);
i) histidine and said codon usage frequency is CAC (0.35) and CAT (0.65);
j) isoleucine and said codon usage frequency is ATA (0.24), ATC (0.36), and ATT (0.40);
k) leucine and said codon usage frequency is CTA (0.12), CTC (0.18), CTG (0.25), CTT (0.12), TTA (0.10), and TTG (0.23);
l) lysine and said codon usage frequency is AAA (0.48) and AAG (0.52);
m) phenylalanine and said codon usage frequency is TTC (0.57) and TTT (0.43);
n) proline and said codon usage frequency is CCA (0.32), CCC (0.24), CCG (0.12), and CCT (0.32);
o) serine and said codon usage frequency is AGC (0.19), AGT (0.13), TCA (0.21), TCC (0.26), TCG (0.06), and TCT (0.15);
p) threonine and said codon usage frequency is ACA (0.36), ACC (0.27), ACG (0.06) and ACT (0.31);
q) tyrosine and said codon usage frequency is TAC (0.41) and TAT (0.59), or
r) valine and said codon usage frequency is GTA (0.15), GTC (0.26), GTG (0.36), and GTT (0.23).

36. The method of claim 29, wherein said one or more plant viruses are dicotyledonous plant viruses.

37. The method of claim 36, wherein said type of amino acid is
a) alanine and said codon usage frequency is GCA (0.33), GCC (0.21), GCG (0.13), and GCT (0.33);
b) arginine and said codon usage frequency is AGA (0.34), AGG (0.23), CGA (0.11), CGC (0.09), CGG (0.08), and CGT (0.15);
c) asparagine and said codon usage frequency is AAC (0.41) and AAT (0.59);
d) aspartic acid and said codon usage frequency is GAC (0.37) and GAT (0.63);
e) cysteine and said codon usage frequency is TGC (0.41) and TGT (0.59);
f) glutamine and said codon usage frequency is CAA (0.60) and CAG (0.40);
g) glutamic acid and said codon usage frequency is GAA (0.61) and GAG (0.39);
h) glycine and said codon usage frequency is GGA (0.35), GGC (0.18), GGG (0.18), and GGT (0.29);
i) histidine and said codon usage frequency is CAC (0.43) and CAT (0.57);
j) isoleucine and said codon usage frequency is ATA (0.31), ATC (0.28), and ATT (0.41);
k) leucine and said codon usage frequency is CTA (0.12), CTC (0.14), CTG (0.12), CTT (0.19), TTA (0.22), and TTG (0.21);
l) lysine and said codon usage frequency is AAA (0.54) and AAG (0.46);
m) phenylalanine and said codon usage frequency is TTC (0.44) and TTT (0.56);
n) proline and said codon usage frequency is CCA (0.38), CCC (0.18), CCG (0.12), and CCT (0.31);
o) serine and said codon usage frequency is AGC (0.14), AGT (0.20), TCA (0.23), TCC (0.14), TCG (0.08), and TCT (0.21);
p) threonine and said codon usage frequency is ACA (0.36), ACC (0.20), ACG (0.14) and ACT (0.31);
q) tyrosine and said codon usage frequency is TAC (0.41) and TAT (0.59); or
r) valine and said codon usage frequency is GTA (0.19), GTC (0.21), GTG (0.25), and GTT (0.35).

38. The method of claim 36, wherein said usage frequency in one or more plant viruses is based on nucleic acid molecules encoding dicotyledonous plant virus coat polypeptides and capsid polypeptides.

39. The method of claim 38, wherein said type of amino acid is
a) alanine and said codon usage frequency is GCA (0.24), GCC (0.27), GCG (0.15), and GCT (0.34);
b) arginine and said codon usage frequency is AGA (0.24), AGG (0.22), CGA (0.12), CGC (0.10), CGG (0.11), and CGT (0.21);
c) asparagine and said codon usage frequency is AAC (0.44) and AAT (0.56);
d) aspartic acid and said codon usage frequency is GAC (0.32) and GAT (0.68);
e) cysteine and said codon usage frequency is TGC (0.25) and TGT (0.75);
f) glutamine and said codon usage frequency is CAA (0.59) and CAG (0.41);
g) glutamic acid and said codon usage frequency is GAA (0.61) and GAG (0.39);
h) glycine and said codon usage frequency is GGA (0.32), GGC (0.20), GGG (0.18), and GGT (0.30);
i) histidine and said codon usage frequency is CAC (0.35) and CAT (0.65);
j) isoleucine and said codon usage frequency is ATA (0.39), ATC (0.26), and ATT (0.35);
k) leucine and said codon usage frequency is CTA (0.10), CTC (0.13), CTG (0.12), CTT (0.14), TTA (0.28), and TTG (0.23);
l) lysine and said codon usage frequency is AAA (0.45) and AAG (0.55);
m) phenylalanine and said codon usage frequency is TTC (0.47) and TTT (0.53);
n) proline and said codon usage frequency is CCA (0.27), CCC (0.27), CCG (0.14), and CCT (0.33);
o) serine and said codon usage frequency is AGC (0.15), AGT (0.19), TCA (0.18), TCC (0.14), TCG (0.11), and TCT (0.24);
p) threonine and said codon usage frequency is ACA (0.25), ACC (0.25), ACG (0.16) and ACT (0.34);
q) tyrosine and said codon usage frequency is TAC (0.37) and TAT (0.63), or
r) valine and said codon usage frequency is GTA (0.17), GTC (0.23), GTG (0.25), and GTT (0.35).

40. The method of claim 36, wherein said one or more dicotyledonous plant viruses is a soybean-specific virus.

41. The method of claim 1, wherein said polypeptide is an insecticidal polypeptide.

42. The method of claim 41, wherein said insecticidal polypeptide is a codon optimized polypeptide based on a polypeptide from Bacillus thuringiensis or Rhyzopus oryzae.

43. The method of claim 42, wherein said insecticidal Bacillus thuringiensis polypeptide is 437N.

44. The method of claim 43, wherein said codon optimized polypeptide insecticidal Bacillus thuringiensis polypeptide comprises the amino acid sequence of SEQ ID NO:2.

45. The method of claim 42, wherein said insecticidal Rhyzopus oryzae polypeptide is insecticidal lipase.

46. The method of claim 45, wherein said codon optimized insecticidal Rhyzopus oryzae polypeptide comprises the amino acid sequence of SEQ ID NO:4.

47. A nucleic acid molecule comprising at least one altered codon wherein said nucleic acid molecule is designed according to the method of claim 1.

48. The nucleic acid molecule of claim 47, wherein said nucleic acid molecule encodes an insecticidal polypeptide.

49. The nucleic acid molecule of claim 48, wherein said insecticidal polypeptide is a codon optimized polypeptide based on a polypeptide from Bacillus thuringiensis or Rhyzopus oryzae.

50. The nucleic acid molecule of claim 49, wherein said insecticidal Bacillus thuringiensis polypeptide is 437N.

51. The nucleic acid molecule of claim 50, wherein said codon optimized insecticidal Bacillus thuringiensis polypeptide comprises the sequence of SEQ ID NO:1.

52. The nucleic acid molecule of claim 49, wherein said insecticidal Rhyzopus oryzae polypeptide is insecticidal lipase.

53. The nucleic acid molecule of claim 52, wherein said codon optimized insecticidal Rhyzopus oryzae polypeptide comprises the sequence of SEQ ID NO:3.

54. A nucleic acid molecule comprising SEQ ID NO:1 or compliment thereof.

55. A nucleic acid molecule comprising SEQ ID NO:3 or compliment thereof.

56. A vector comprising the nucleic acid molecule according to any of claims 53 or 54.

57. A transgenic plant and progeny thereof comprising the nucleic acid molecule of claim 56.

58. A transgenic plant of claim 57, wherein said progeny are seeds.

59. The transgenic plant of claim 57, wherein said transgenic plant is a monocotyledonous plant.

60. The transgenic plant of claim 59, wherein said transgenic plant is selected from the group consisting of barley, maize, millet, oats, rice, and wheat.

61. The transgenic plant of claim 57, wherein said transgenic plant is a dicotyledonous plant.

62. The transgenic plant of claim 61, wherein said transgenic plant is selected from the group consisting of potato, soybean, tobacco, cotton, and tomato.
